(19) United States
(12) Patent Application Publication
(10) Pub. No.: US 2022/0119870 A1
(43) Pub. Date: Apr. 21, 2022

(54) METHODS FOR DIGITAL MULTIPLEXING OF NUCLEIC ACIDS IN SITU

(71) Applicant: ADVANCED CELL DIAGNOSTICS, INC., Newark, CA (US)

(72) Inventors: Xiao-Jun MA, Newark, CA (US); Emily PARK, Newark, CA (US); Shiping CHEN, Newark, CA (US)

(21) Appl. No.: 17/285,825

(22) PCT Filed: Oct. 31, 2019
(86) PCT No.: PCT/US2019/059204

§ 371 Date: Apr. 15, 2021

Related U.S. Application Data
(60) Provisional application No. 62/754,427, filed on Nov. 1, 2018.

(51) Int. Cl. C12Q 1/6841; C12Q 1/6876; C12Q 1/682

(52) U.S. Cl. CPC C12Q 1/6841; C12Q 2600/16; C12Q 1/682; C12Q 1/6876

(57) ABSTRACT

The invention relates to methods of multiplex detection of a plurality of target nucleic acids using combinations of labels by contacting a sample comprising a cell comprising a plurality of target nucleic acids with a set of probes, wherein the set of probes comprises subsets of probes comprising a plurality detectable labels that provide unique labeling of each target nucleic acid, wherein each probe subset comprises one or more distinct labels, wherein the number and/or combination of distinct labels is unique for each target nucleic acid; and detecting the detectable labels bound to the respective target nucleic acids. The invention also relates to samples, slides and kits for multiplex detection of target nucleic acids.

This application claims the benefit of U.S. Provisional application No. 62/754,427 filed Nov. 1, 2018, the entire contents of which are incorporated herein by reference.

### BACKGROUND OF THE INVENTION

The present invention relates generally to detection of nucleic acids, and more specifically to multiplex detection of nucleic acids.

RNA in situ hybridization (ISH) is a molecular biology technique widely used to measure and localize specific RNA sequences, for example, messenger RNAs (mRNAs), long non-coding RNAs (lncRNAs), and microRNAs (miRNAs) within cells, such as circulating tumor cells (CTCs) or tissue sections, while preserving the cellular and tissue context. RNA ISH therefore provides for spatial-temporal visualization as well as quantification of gene expression within cells and tissues. It has wide applications in research and in diagnostics (Hu et al., Biomark. Res. 2(1):1-13, doi: 10.1186/2050-7771-2-3 (2014); Ratan et al., Cureus 9(6):e1325. doi: 10.7759/cureus.1325 (2017); Weier et al., Expert Rev. Mol. Diagn. 2(2):109-119 (2002))). Fluorescent RNA ISH utilizes fluorescent dyes and fluorescent microscopes for RNA labeling and detection, respectively. Fluorescent RNA ISH typically provides for limited multiplexing of four to five target sequences. The limited multiplexing capability is largely due to the small number of spectrally distinct fluorescent dyes that can be distinguished by the optical systems of the fluorescence microscope. Higher level of multiplexing is highly desirable in areas such as generating cell and tissue maps to understand complex biological systems, particularly in human health and disease.

Thus, there exists a need for in situ detection methods for multiplex detection of nucleic acids. The present invention satisfies this need and provides related advantages as well.

### SUMMARY OF INVENTION

The invention provides a method for multiplex detection of a plurality of target nucleic acids in a cell. In one embodiment, the invention provides a method for multiplex detection of a plurality of target nucleic acids in a cell, comprising contacting a sample comprising a cell comprising a plurality of target nucleic acids with a set of probes, wherein the set of probes comprises subsets of probes comprising a plurality detectable labels that provide unique labeling of each target nucleic acid, wherein each probe subset comprises one or more distinct labels, wherein the number and/or combination of distinct labels is unique for each target nucleic acid; and detecting the detectable labels bound to the respective target nucleic acids.

### BRIEF DESCRIPTION OF THE DRAWINGS

FIGS. 1A and 1B show exemplary configurations of probes for detecting a target nucleic acid. In the configuration shown in FIG. 1A, each individual target probe has a target anchor segment (TA) complementary to the target nucleic acid (i.e., a segment of the target probe that can hybridize to the target nucleic acid) and a signal anchor segment (SA) complementary to a component of a Signal Generating Complex (SGC) (i.e., a segment of the target probe that can hybridize to a component of the SGC). Each SGC comprises multiple layers of components, such as amplifiers (AP) and pre-amplifiers (PA) that assemble into a tree-like structure which is capable of carrying many label probes (LP) on its “branches”. As shown in FIG. 1B, if the target sequence is sufficiently long, many TP sets and associated SGCs can be assembled on the target nucleic acid to generate a detectable signal that appears in an imaging system as a discrete “dot”.

FIGS. 2A and 2B show two exemplary configurations of probes for detecting a target nucleic acid. The configurations utilize SGCs comprising LPs, APs, PAs, TPs, as shown in FIG. 1, except that assembly of the SGC utilizes a Collaboration Amplifier (COM). The COMs bind to two pre-amplifiers (PAs) and to an amplifier (AP) for assembly of the SGC. FIGS. 2A and 2B show two different configurations of the target probe binding to the target nuclei acid. The configurations allow more LPs to be incorporated into one SGC. Such configurations are more suitable for detecting short target sequences because a detectable signal can be generated with a single SGC.

FIG. 3 shows a configuration for multiplex detection of target nucleic acids. As shown in FIG. 3, for a multiplex assay with 2^{N}−1 multiplexing channels (i.e., N unique labels), N unique, label-specific SGCs can be made. Each SGC carries the same LPs with a specific label, where different SGCs carry distinct labels. Components of each SGC (such as PAs, APs, LPs, etc.) are uniquely associated with the SGC. The SGCs are designed so that the components of a target-specific SGC hybridize to each other to assemble the SGC but cannot cross-hybridize to any components of any other SGC. In FIG. 3, two target nucleic acids are shown bound to the respective SGCs. For one target nucleic acid (upper target in FIG. 3), the code for the target nucleic acid is 1111, where the SGCs for the target comprise four labels (4, 3, 2, and 1). For a second target (lower target in FIG. 3), the code for the target nucleic acid is 1010, where the SGCs for the target nucleic acid comprise two labels (4 and 2).

FIGS. 4A-4H illustrate various exemplary embodiments for multiplex detection of target nucleic acids. FIG. 4A illustrates one embodiment of sub-SGC implementation, where the SGC ID code is implemented on the amplifier (AP) molecule. As shown in FIG. 4A, an AP has one region designed to bind to the amplifier anchor (AA) on the PA molecule (i.e., binding site for the amplifier on the pre-amplifier) and another region comprising multiple segments of label probe anchors (LAs) (i.e., binding sites for the label probes). In this embodiment, a mixture of different LAs are designed according to the unique identification code of the SGC. For example, if the ID code of the SGC is L4, L3, L2, L1=1110, then equal number of LAs for LP type 4, 3, 2 are made on the AP molecule, which will bind a designed number of desired LPs to generate the ID code in the assay. In FIG. 4A, the code for the SGC shown is 1101 using labels 4, 3 and 1.

FIG. 4B illustrates another embodiment of sub-SGC implementation, where the SGC ID code is implemented on the pre-amplifier (PA) molecule. As shown in FIG. 4B, N “pure” AP molecules are made, each carrying the same type of LP. A PA molecule has one region designed to bind to the SA (i.e., binding site for the PA on the TP; see FIG. 1A) of a TP set and another region comprising multiple segments of AAs (i.e., binding sites for amplifiers on the pre-amplifier). In this embodiment, a mixture of different AAs are designed according to the unique identification code of the SGC. For example, if the ID code of the SGC is L4, L3, L2, L1=1010, then equal number of AAs for APs carrying LP type 4, 2 are made on the PA molecule, which will bind a designed number of desired LPs to generate the ID code in the assay. In FIG. 4B, the code for the SGC shown is 1101 using labels 4, 3 and 1.

FIG. 4C illustrates another embodiment of sub-SGC implementation, where the SGC ID code is implemented on the LP molecule. In this embodiment, LP molecules binding to the same SGC can be mixtures of LPs each conjugated to a different label according to a predefined code book. For example, as shown in FIG. 4C, SGC5 LPs are a mixture of LPs conjugated to three different labels, creating the ID code 1101, corresponding to labels 4, 3 and 1. The advantage of this embodiment is that the “coloring” of the SGC complex by the LPs will be completely randomized, which can further help to reduce coding errors. A partial LP mixing code book is shown on the right of FIG. 4C, with 7 different exemplary SGC codes shown using 4 labels.

FIG. 4D shows an embodiment of FIG. 4C in more detail. For each SGC, a specific label anchor (LA, the binding site on the amplifier for the label probe) is assigned so that each SGC for a particular target nucleic acid has a plurality of the same LAs on the amplifier. The level at which combinatorial labeling can be provided is with the label probes (LPs). In this case, SGC5 is illustrated showing that the amplifiers comprise a plurality of identical LAs, labeled “E.” As shown in FIG. 4D, SGC5 is coded with 3 ID codes (1101) corresponding to 3 distinct label probes (4, 3 and 1), all of which have the same binding site for the plurality of “E” LAs on the corresponding amplifier. Therefore, all three label probes (4, 3 and 1) are bound to the amplifiers of SGC5, thereby labeling the SGC5 target nucleic acid with the label code 1101.

FIG. 4E shows an embodiment of FIG. 4C in more detail. The SGC5 of FIG. 4D is shown bound to its respective target nucleic acid, with the label probes having an “E” binding site bound to the respective “E” LAs of the SGC5 amplifiers. Also shown are two additional exemplary SGCs bound to their respective target nucleic acids. SGC1, coded as shown in FIG. 4D, comprises a plurality of identical LAs, labeled “A.” SGC1 is coded with 1 ID code (0001) corresponding to a label probe (1), which has the binding site for the plurality of “A” LAs of the SGC1 amplifiers. Therefore, the label probe “1” is bound to the amplifiers of SGC1, thereby labeling the SGC1 target nucleic acid with the label code 0001. SGC3, coded as shown in FIG. 4D, comprises a plurality of identical LAs, labeled “C.” SGC3 is coded with 2 ID probes (0011) corresponding to 2 distinct label probes (2 and 1), both of which have the same binding site for the plurality of “C” LAs on the corresponding SGC3 amplifiers. Therefore, both label probes (2 and 1) are bound to the amplifiers of SGC3, thereby labeling the SGC3 target nucleic acid with the label code 0011.

FIG. 4F shows an embodiment of FIG. 4C in more detail. FIG. 4F illustrates that, once an SGC for a particular target nucleic acid has been designed, the actual coding for the target nucleic acid can be readily modified simply by changing the labels on the label probes that bind to the amplifiers of a particular SGC. For example, in FIG. 4D, SGC2 comprises amplifiers with “B” LAs and is coded as 0010 using label 2. In FIG. 4F, the same SGC assembly can be used with respect to the target probes, pre-amplifier, and amplifiers with “B” LAs, but instead of using “B” LA-binding label probes with only label 2 as in FIG. 4D, “B” LA-binding label probes can be used that have a mixture of labels 3 and 2 such that SGC2 is now coded with both labels (0110). Thus, labels 3 and 2 (0110) are bound to “B” LAs on the SGC2 amplifiers. Similarly, SGCS comprising amplifiers with “E” LAs is now coded in FIG. 4F as 1110 by using label probes with “E” LA-binding label probes that have a mixture of labels 4, 3 and 2 (1110), instead of labels 4, 3 and 1 (1101) as shown in FIG. 4D.

FIG. 4G shows an embodiment of FIG. 4C in more detail. In FIG. 4G, an additional “coding” can be implemented by using different ratios of label probes. As shown in FIG. 4G, rather than binding the distinct label probes in equivalent amounts, the ratio of distinct label probes bound to the corresponding amplifiers can be varied such that not only the presence of a particular label but also the relative amount of a particular label can be used as another way to provide a distinct label. As shown in FIG. 4G, SGC2 is coded as 0110 with labels 3 and 2, whereas SGC2′ can be used to code a different target nucleic acid using the code 011′0 and the same labels 3 and 2, but where the ratio of label 2 to label 3 bound to SGC2′ is different than the ratio of label 2 to label 3 bound to SGC2. The two target nucleic acids are labeled with the same label probes, but the target nucleic acids can be distinguished based on the relative amounts of the two label probes bound to the respective target nucleic acids.

FIG. 4H shows an embodiment of FIG. 4C in more detail. As shown in FIG. 4H, two target nucleic acids are shown with two bound SGCs, SGC2 and SGC5. SGC2 is coded as 0110 with labels 3 and 2, and SGC5 is coded as 1110 with labels 4, 3 and 2. In this case, where all of the label probes bind to the respective LAs, “B” LAs in the case of SGC2 and “E” LAs in the case of SGC5, and assuming that the SGC2 and SGC5 have approximately the same number of LAs in the respective SGCs, the number of respective labels that can bind to SGC2 will be higher than the number of respective labels that bind to SGC5 (i.e., the 2 distinct labels for SGC2 (labels 3 and 2) and the 3 distinct labels for SGC5 (labels 4, 3 and 2) will be bound to the same number of sites, resulting in a higher number of labels 3 and 2 being bound to SGC2 than SGC5 since some of the SGC5 sites are occupied by label 4). If desired, the number of labels (and therefore intensity of signal) can be normalized by including “blank” label probes, i.e., probes having a binding site for the respective LAs (in this case “B” for SGC2 and “E” for SGC5) but without a label. For example, if it is desired to compare SGC2 and SGC5 with equal intensity signals for the respective labels, ⅓ “blank” label probes can be included with the mixture of “B” LA-specific probes so that the intensity of labels 3 and 2 will be the same on both SGCs (i.e., ⅓ of SGC2 occupied by “blank” label probes and ⅓ of SGC5 occupied by label 4). In another example, if a multiplex assay is being performed where some SGCs include 4 labels, then the assay can be performed so that the same proportion of “blank” label probes are included in the label probe sets using less than 4 labels, for example, ½ “blank” label probes can be included with the SG2-specific label probes and ¼ “blank” label probes to be included with the SGC5-specific label probes so that the amount of each distinct label probe, 4, 3, 2 and 1, bound to the respective SGCs is the same on each SGC.

FIG. 5 shows two configurations of assembly of SGCs on a target nucleic acid. In the lower panel of FIG. 5, the SGCs providing the same label are shown binding in a group next to each other. In the upper panel of FIG. 5, the SGCs providing different labels are shown with binding sites on the target nucleic acid intermingled or intertwined. The intermingling of target probe binding sites on the target nucleic acid for different labels are advantageous because, if different SGC types are positioned apart, in separate groups, a certain section of the target may be blocked or masked, thereby preventing attachment of one specific SGC type, which will result in miscoding.

FIGS. 6A and 6B demonstrate configurations for reducing miscoding for multiplex detection of target nucleic acids. As shown in FIG. 6A, the particular SGC is miscoded from “1001” to “0001” because the PA is truncated, which could occur during manufacturing of the PA. In this case in FIG. 6A, label 4 is not bound due to truncation. As shown in FIG. 6B, the same truncation will not cause miscoding if the labels are intertwined or intermingled on the PA. Arranging different labels into alternating positions reduces the chance of miscoding.

FIGS. 7A and 7B show a method to minimize potential miscoding caused by truncation by randomizing the position of different labels on the AP or PA. As illustrated in FIG. 7, the multiplexing channel ID is encoded on the AP molecule. In FIG. 7A, different label probes are positioned on each AP in exactly the same way, that is, each amplifier in the SGC is the same. Truncation of some of the APs can cause substantial reduction in certain labels being bound to the target nucleic acid compared to other labels on different positions of the AP. This imbalance increases the chance of miscoding. In the most severe case, truncation could cause the loss of all copies of one certain label, leading to an outright miscode. In FIG. 7B, locations of different labels on the APs are randomized. The APs are provided as a plurality of amplifiers, where a mix of non-identical amplifiers is included, where the position of LAs for specific label probes are distributed differently and can be randomized on the non-identical amplifiers. Truncation therefore does not cause a large bias in the number of labels in the SGC.

FIGS. 8A-8F show implementation of the multiplex detection strategy depicted in FIG. 4C. Three fluorescent dyes (Alexa488, ATTO550 and ATTO647N) were used to detect four target mRNAs, 5-hydroxytryptamine receptor 7 (Htr7), protocadherin 8 (Pcdh8), tyrosine hydroxylase (Th), and forkhead box P1 (Foxp1). The assay was performed on frozen mouse brain section using the RNAscope® HiPlex assay (acdbio.com/rnascope-hiplex-assays). The fluorescent codes for each target was as follows: Htr7, 1000 (Alexa488), Pcdh8, 0100 (ATTO550), Th, 1100 (Alexa488, ATTO550) and Foxp1, 1010 (Alexa488, ATTO647N). FIG. 8A shows an overview of stained mouse brain section. The boxed region in FIG. 8A is shown with 40× magnification in FIG. 8B. The zoomed image was processed with the Richardson-Lucy spatial deconvolution algorithm in MATLAB (Mathworks; Natick, Mass.), signal dots were detected (exemplary signal dots shown with arrows labeled 801-804), and colors were decoded to individual targets and shown in FIGS. 8C-8F. Nuclei were stained with DAPI (exemplary staining labeled 805).

FIGS. 9A-9C show a schematic of previously described methods of detecting a nucleic acid target using a signal generating complex (SGC). PPA, pre-pre-amplifier; PA, pre-amplifier; AMP, amplifier; LP, label probe.

FIGS. 10A-10C show a schematic of orthogonal labeling of target nucleic acids. Shown in FIG. 10A is orthogonal labeling of target nucleic acids based on an RNAscope™ assay. Shown in FIG. 10A is the labeling of three exemplary target nucleic acids with respective signal generating complexes (SGCs). FIG. 10A shows the binding of target probe pair 1 (TP1a and TP1b) to target nucleic acid 1. Pre-amplifier (PA1) is shown bound to the target probe pair (TP1a and TP1b). A plurality of amplifiers (AMP1) is shown bound to PA1. A plurality of label probes (LP1) is shown bound to the amplifiers. FIG. 10A shows a similar configuration for targets 2 and 3, with the components of the SGC (target probes, pre-amplifiers, amplifiers, label probes) specific for each of the respective targets. FIG. 10B shows a modification of the configuration shown in FIG. 10A. Shown in FIG. 10B is the labeling of two exemplary target nucleic acids with respective signal generating complexes (SGCs). FIG. 10B shows the binding of target probe pair 1 (TP1a and TP1b) to target nucleic acid 1. Pre-pre-amplifier (PPA1) is shown bound to the target probe pair (TP1a and TP1b). A plurality of pre-amplifiers (PA1) is shown bound to PPA1. A plurality of amplifiers (AMP1) is shown bound to PA1. The amplifiers are shown bound to one pre-amplifier for simplicity, but it is understood that the amplifiers can be bound to all of the pre-amplifiers. A plurality of label probes (LP1) is shown bound to the amplifiers. FIG. 10B shows a similar configuration for target 2, with the components of the SGC (target probes, pre-pre-amplifiers, pre-amplifiers, amplifiers, label probes) specific for each of the respective targets. Shown in FIG. 10C is orthogonal labeling of target nucleic acids based on a Basescope™ assay. Shown in FIG. 10C is the labeling of two exemplary target nucleic acids with respective signal generating complexes (SGCs). FIG. 10C shows the binding of target probe pair 1 (TP1a and TP1b) to target nucleic acid 1. A pair of pre-pre-amplifiers (PPA1a and PPA1b) are shown bound to respective target probe pairs (TP1a and TP1b). Pre-amplifier (PA1) is shown bound to the pre-pre-amplifier pairs (PPA1a and PPA1b). A plurality of amplifiers (AMP1) is shown bound to PA1. The amplifiers are shown bound to one pre-amplifier for simplicity, but it is understood that the amplifiers can be bound to all of the pre-amplifiers. A plurality of label probes (LP1) is shown bound to the amplifiers. FIG. 10C shows a similar configuration for target 2, with the components of the SGC (target probes, pre-pre-amplifiers, pre-amplifiers, amplifiers, label probes) specific for each of the respective targets.

### DETAILED DESCRIPTION OF THE INVENTION

The present invention relates to methods for multiplex analysis of nucleic acids, for example, by in situ hybridization. The methods of the invention allow the detection of multiple target nucleic acids within the same sample and within the same cell. The methods of the invention utilize labeling of target nucleic acids with combinations of labels that uniquely identify the target nucleic acids.

As used herein, the term “label probe” refers to an entity that binds to a target molecule, directly or indirectly, generally indirectly, and allows the target to be detected. A label probe (or “LP”) contains a nucleic acid binding portion that is typically a single stranded polynucleotide or oligonucleotide that comprises one or more labels which directly or indirectly provides a detectable signal. The label can be covalently attached to the polynucleotide, or the polynucleotide can be configured to bind to the label. For example, a biotinylated polynucleotide can bind a streptavidin-associated label. The label probe can, for example, hybridize directly to a target nucleic acid. In general, the label probe can hybridize to a nucleic acid that is in turn hybridized to the target nucleic acid or to one or more other nucleic acids that are hybridized to the target nucleic acid. Thus, the label probe can comprise a polynucleotide sequence that is complementary to a polynucleotide sequence, particularly a portion, of the target nucleic acid. Alternatively, the label probe can comprise at least one polynucleotide sequence that is complementary to a polynucleotide sequence in an amplifier, pre-amplifier, pre-pre-amplifier, signal generating complex (SGC), or the like, as described herein. In general in embodiments of the invention, the label probe binds to an amplifier. As used herein, a label probe comprising an enzyme label refers to a label probe comprising a nucleic acid binding portion such as an oligonucleotide and an enzyme that is coupled to the nucleic acid binding portion. As disclosed herein, the coupling of the enzyme to the nucleic acid binding portion can be covalent or through a high affinity binding interaction such as biotin/avidin or other similar high affinity binding molecules.

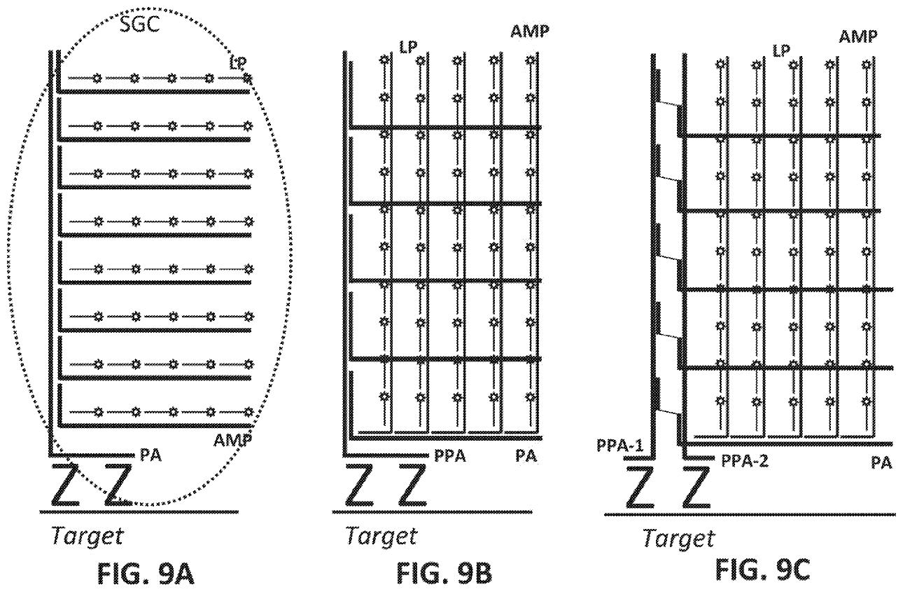

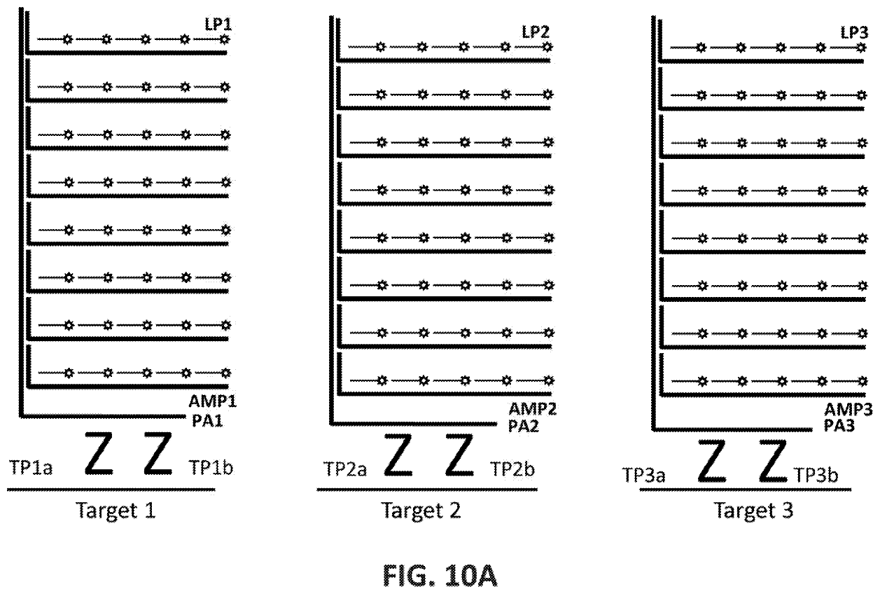

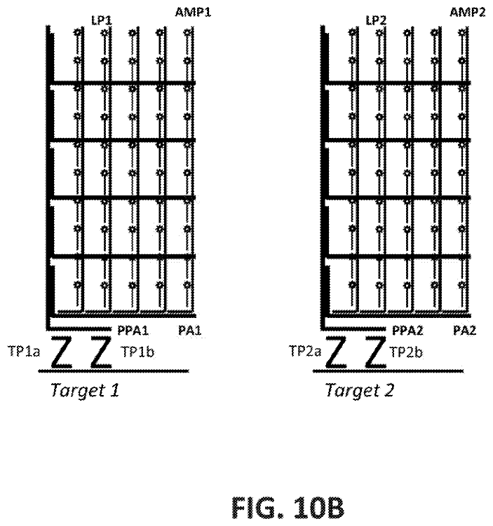

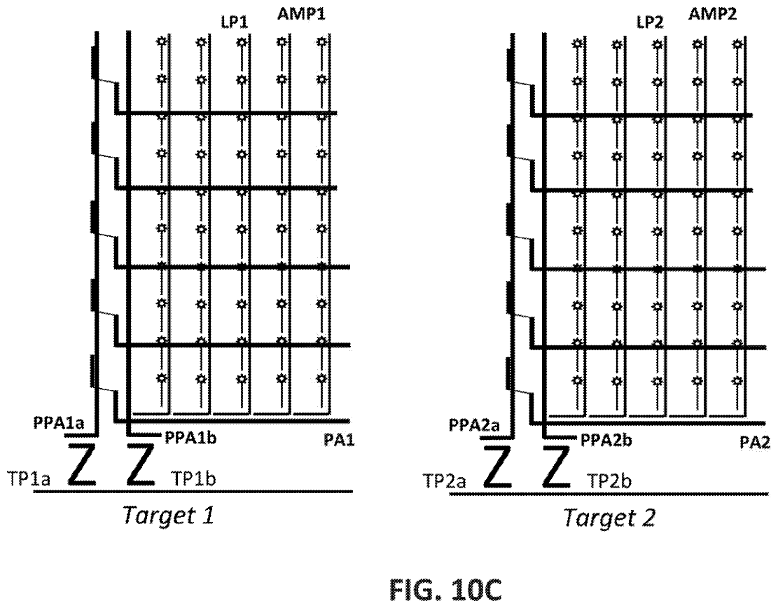

As used herein, a “target probe” is a polynucleotide that is capable of hybridizing to a target nucleic acid and capturing or binding a label probe or signal generating complex (SGC) component, for example, an amplifier, pre-amplifier or pre-pre-amplifier, to that target nucleic acid. The target probe can hybridize directly to the label probe, or it can hybridize to one or more nucleic acids that in turn hybridize to the label probe; for example, the target probe can hybridize to an amplifier, a pre-amplifier or a pre-pre-amplifier in an SGC. The target probe thus includes a first polynucleotide sequence that is complementary to a polynucleotide sequence of the target nucleic acid and a second polynucleotide sequence that is complementary to a polynucleotide sequence of the label probe, amplifier, pre-amplifier, pre-pre-amplifier, or the like. In general in embodiments of the invention, the target probe binds to a pre-amplifier, as in FIGS. 9A and 10A, or to a pre-pre-amplifier, as in FIGS. 9B, 9C, 10B and 10C. The target probe is generally single stranded so that the complementary sequence is available to hybridize with a corresponding target nucleic acid, label probe, amplifier, pre-amplifier or pre-pre-amplifier. In embodiments of the invention, the target probes are provided as a pair.

As used herein, an “amplifier” is a molecule, typically a polynucleotide, that is capable of hybridizing to multiple label probes. Typically, the amplifier hybridizes to multiple identical label probes. The amplifier can also hybridize to a target nucleic acid, to at least one target probe of a pair of target probes, to both target probes of a pair of target probes, or to nucleic acid bound to a target probe such as an amplifier, pre-amplifier or pre-pre-amplifier. For example, the amplifier can hybridize to at least one target probe and to a plurality of label probes, or to a pre-amplifier and a plurality of label probes. In general in embodiments of the invention, the amplifier can hybridize to a pre-amplifier. The amplifier can be, for example, a linear, forked, comb-like, or branched nucleic acid. As described herein for all polynucleotides, the amplifier can include modified nucleotides and/or nonstandard internucleotide linkages as well as standard deoxyribonucleotides, ribonucleotides, and/or phosphodiester bonds. Suitable amplifiers are described, for example, in U.S. Pat. Nos. 5,635,352, 5,124,246, 5,710,264, 5,849,481, and 7,709,198 and U.S. publications 2008/0038725 and 2009/0081688, each of which is incorporated by reference. In general in embodiments of the invention, the amplifier binds to a pre-amplifier and label probes (see FIGS. 9 and 10).

As used herein, a “pre-amplifier” is a molecule, typically a polynucleotide, that serves as an intermediate binding component between one or more target probes and one or more amplifiers. Typically, the pre-amplifier hybridizes simultaneously to one or more target probes and to a plurality of amplifiers. Exemplary pre-amplifiers are described, for example, in U.S. Pat. Nos. 5,635,352, 5,681,697 and 7,709,198 and U.S. publications 2008/0038725, 2009/0081688 and 2017/0101672, each of which is incorporated by reference. In general in embodiments of the invention, a pre-amplifier binds to both members of a target probe pair (see FIGS. 9A and 10A), to a pre-pre-amplifier that can bind to a target probe pair (FIGS. 9B and 10B), or to both members of a pair of pre-pre-amplifiers that can bind to a target probe pair (see FIGS. 9C and 10C). A pre-amplifier also binds to an amplifier (see FIGS. 9 and 10).

As used herein, a “pre-pre-amplifier” is a molecule, typically a polynucleotide, that serves as an intermediate binding component between one or more target probes and one or more pre-amplifiers. Typically, the pre-pre-amplifier hybridizes simultaneously to one or more target probes and to a plurality of pre-amplifiers. Exemplary pre-pre-amplifiers are described, for example, in 2017/0101672, which is incorporated by reference. In general in embodiments of the invention, a pre-pre-amplifier binds to a target probe pair (see FIGS. 9B and 10B) or to a member of a target probe pair (see FIGS. 9C and 10C) and to a pre-amplifier.

As used herein, the term “plurality” is understood to mean two or more. Thus, a plurality can refer to, for example, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 55 or more, 60 or more, 65 or more, 70 or more, 75 or more, 80 or more, 85 or more, 90 or more, 95 or more, 100 or more, 110 or more, 120 or more, 130 or more, 140 or more, 150 or more, 160 or more, 170 or more, 180 or more, 190 or more, 200 or more, 300 or more, 400 or more, 500 or more, 600 or more, 700 or more, 800 or more, 900 or more, or 1000 or more, or even a greater number, if desired for a particular use.

As described herein, the invention relates to multiplex detection of target nucleic acids, where the methods provide for detection of higher numbers of target nucleic acids than previously described methods of in situ hybridization. The methods can employ orthogonal amplification systems to distinctly detect multiple target nucleic acids.

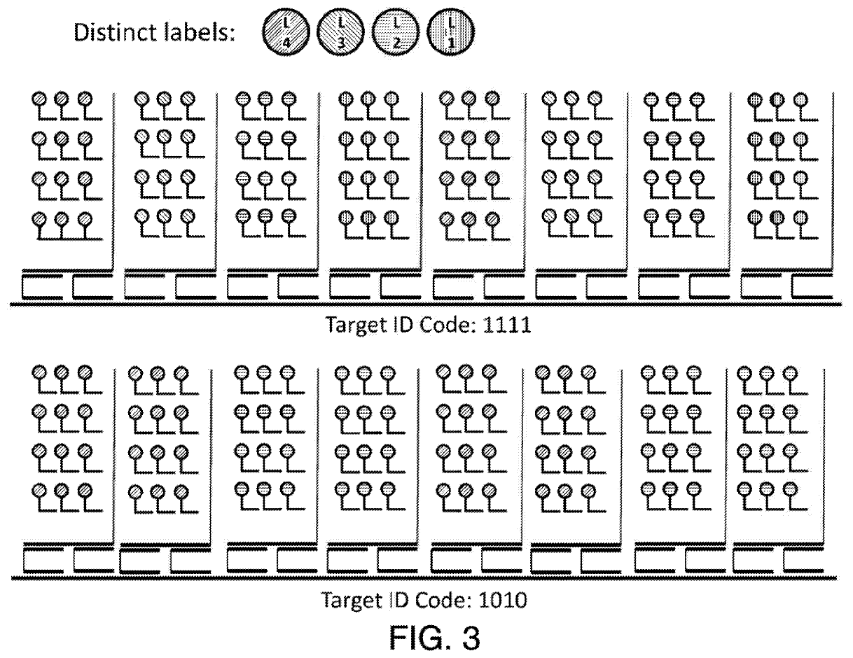

In one embodiment, the invention provides a method for multiplex detection of a plurality of target nucleic acids, comprising contacting a sample comprising a plurality of target nucleic acids with a set of probes, wherein the set of probes comprises subsets of probes comprising one or more probes that are specific for each target nucleic acid, wherein each of the probe subsets comprises a plurality detectable labels that provide a combination of detectable labels, wherein the combination of detectable labels provides unique labeling of each target nucleic acid, and detecting the combination of detectable labels bound to the respective target nucleic acids. In one embodiment of such a method, the combination of detectable labels is selected from the combination of label configurations depicted in any of FIGS. 3-7 or described herein.

In one embodiment, the invention provides a method for multiplex detection of a plurality of target nucleic acids in a cell, comprising (A) contacting a sample comprising a cell comprising a plurality of target nucleic acids with a set of probes, wherein the set of probes comprises subsets of probes comprising a plurality detectable labels that provide unique labeling of each target nucleic acid, wherein each probe subset comprises one or more distinct labels, wherein the number and/or combination of distinct labels is unique for each target nucleic acid; and (B) detecting the detectable labels bound to the respective target nucleic acids.

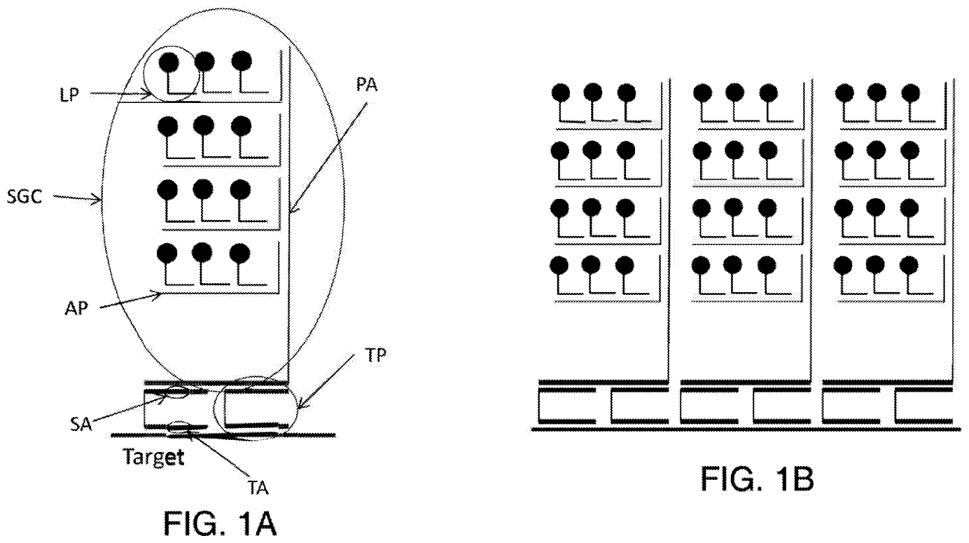

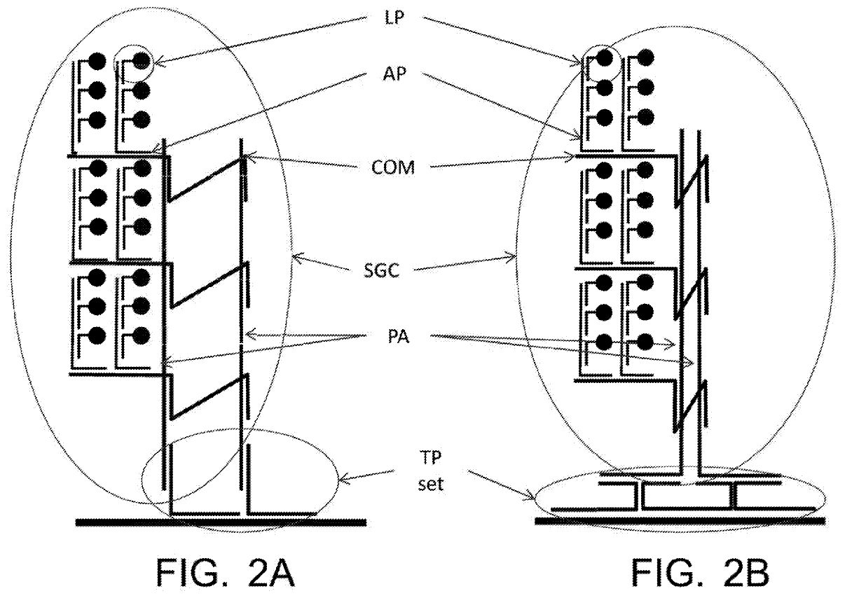

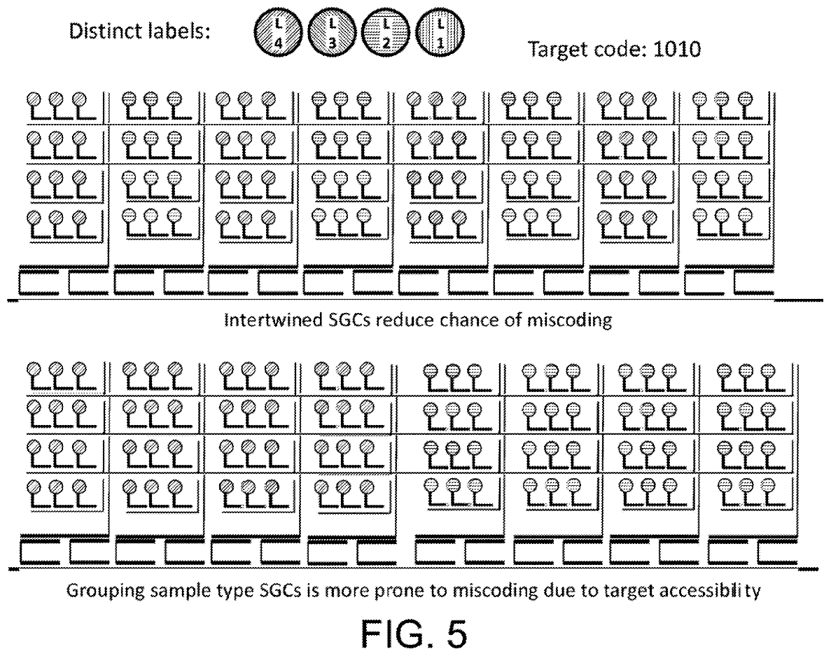

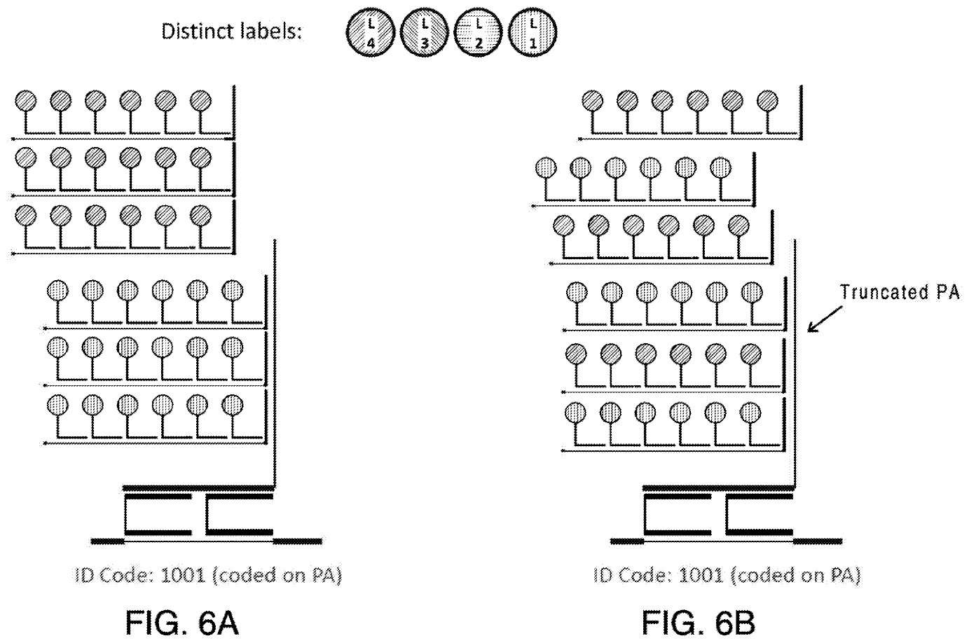

Previously disclosed methods of detecting target nucleic acids are described in U.S. Pat. No. 7,709,198 and European Patent No. 2500439, which describe methods of in situ detection of nucleic acid targets, in which target probes (TP) are arranged in sets of two or more short probes adjacent to each other when they are hybridized to the target. As shown in FIG. 1A, each individual target probe has a target anchor segment (TA) complementary to the target and a signal anchor segment (SA) complementary to a component of a Signal Generating Complex (SGC). Each SGC comprises multiple layers of components, such as Amplifiers (AP) and Pre-Amplifiers (PA) that assemble into a tree-like structure which is capable of carrying many Label Probes (LP) on its “branches”. As shown in FIG. 1B, if the target sequence is sufficiently long, many TP sets and associated SGCs can be assembled on the target to generate a detectable signal that appears in an imaging system as a discrete “dot”. Manual or computerized dot counting can be conducted to quantify the number of targets in a particular cell in a sample. FIG. 2 shows two additional different configurations of the SGC, in which additional layer(s) of amplification molecules, such as a Collaboration Amplifier (COM), are incorporated to carry more LPs in one SGC. Such configurations are more suitable for detecting short target sequences because a detectable signal can be generated with a single SGC (see, for example, WO 2017/066211, which is incorporated herein by reference). A Collaboration Amplifier (COM) is also illustrated in FIGS. 5C and 6C, where the COM is shown as a pre-amplifier, which binds to two pre-pre-amplifiers at multiple sites and to a plurality of amplifiers.

In many applications, it is highly valuable to detect many targets in the same assay. Previously disclosed methods address this need through “pooling” or “multiplexing” approaches. In the pooling approach, each target has unique TP sets but all TP sets have the same SAs and can therefore be bound to the same SGCs. In this way, a signal is detected when any one of the multiple target is present. The pooling approach is useful when a group of targets has the same clinical utility or biological functionality. In the multiplexing approach, each target has its unique SGC that does not cross hybridize, generating a uniquely identifiable signal for each target when it is present. The multiplexing approach is useful when each target in the group provide a different clinical or biological indication alone or in combination. In previously described methods, each unique signal is generated by a large number of LPs carrying the same label. The problem with this approach is that there are usually a limited number of uniquely identifiable labels. In fluorescent detection modalities, for example, four fluorophores at different wavelengths are commonly used. More than six fluorophores in an imaging based multiplexing system is possible but becomes difficult due to bandwidth limits and cross-talk between wavelengths. This limitation imposes a limit on the number of targets that can be multiplexed in an assay. The invention disclosed herein breaks this limitation, thereby enabling a much higher level of multiplexing capability.

In the invention disclosed herein, each LP has a unique label but the LPs associated with a target are not necessarily the same. Instead they can be a mixture of several different LPs that form combinations that are uniquely identifiable. An example is shown in Table 1, where a multiplexed assay with four different labels (L1, L2, L3 and L4) can generate 15 unique combinations or identity codes to detect 15 targets.

TABLE 1
Combinations of distinguishable labeling based on four
distinct labels.

| | | Presence of labels (“1” present, “0” absent) | | | |
|---|---|---|---|---|---|
| | | L4 | L3 | L2 | L1 |
| ID Codes of | T1 | 0 | 0 | 0 | 1 |
| Targets or | T2 | 0 | 0 | 1 | 0 |
| Multiplexing | T3 | 0 | 0 | 1 | 1 |
| Channels | T4 | 0 | 1 | 0 | 0 |
| | T5 | 0 | 1 | 0 | 1 |
| | T6 | 0 | 1 | 1 | 0 |
| | T7 | 0 | 1 | 1 | 1 |
| | T8 | 1 | 0 | 0 | 0 |
| | T9 | 1 | 0 | 0 | 1 |
| | T10 | 1 | 0 | 1 | 0 |
| | T11 | 1 | 0 | 1 | 1 |
| | T12 | 1 | 1 | 0 | 0 |
| | T13 | 1 | 1 | 0 | 1 |
| | T14 | 1 | 1 | 1 | 0 |
| | T15 | 1 | 1 | 1 | 1 |

In this way, up to 2^{N}−1 unique multiplexing channels can be created using N unique labels. Since each molecule of a specific target appears as a discrete dot in an image and the label composition of the dot determines the identity of the target, this “digital” multiplexing scheme does not affect quantification of targets and is capable of tolerating a high level of noise.

The digital multiplexing scheme described above can be implemented at the SGC level if the target sequence is sufficiently long. As shown in FIG. 3, for a multiplex assay with 2^{N}−1 multiplexing channels (i.e., N unique labels), N unique, label-specific SGCs can be made. Each SGC carries the same LP with a specific label. Components of each SGC (such as PAs, APs, LPs, etc.) are uniquely associated with the SGC. They are specially designed to hybridize to each other to assemble the SGC but cannot cross-hybridize to any components of any other SGC. In the exemplary embodiment shown in FIG. 3, two target nucleic acids are shown bound to the respective SGCs. For one target nucleic acid (upper target in FIG. 3), the code for the target nucleic acid is 1111, where the SGCs for the target comprise four labels (4, 3, 2, and 1). For a second target (lower target in FIG. 3), the code for the target nucleic acid is 1010, where the SGCs for the target nucleic acid comprise two labels (4 and 2). When the target sequence is long, many TP sets can be designed to bind specifically to the target. Each TP set can be coupled to a selected SGC through their SAs (see FIG. 1). Many SGCs are captured to each target in this way. Each target is uniquely identifiable in the assay through its unique combination of SGCs. The advantage of SGC level implementation is that only a relatively small number of different SGCs need to be developed. Since it is essential that components of each SGC do not cross-hybridize to other SGCs, the amount of work involved in developing a large number of SGCs is substantial. The disadvantage of this embodiment is that the length of target sequence has to be sufficiently long to accommodate many SGCs. In addition, some SGCs may not be assembled successfully on the target due to accessibility of the target sequence. This may lead to miscoding, which can be addressed as described below.

In one embodiment, the invention provides a method for multiplex detection of a plurality of target nucleic acids in a cell, comprising (A) contacting a sample comprising a cell comprising a plurality of target nucleic acids with a set of probes, wherein the set of probes comprises subsets of probes comprising a plurality detectable labels that provide unique labeling of each target nucleic acid, wherein each probe subset comprises one or more distinct labels, wherein the number and/or combination of distinct labels is unique for each target nucleic acid; and (B) detecting the detectable labels bound to the respective target nucleic acids; and wherein each probe in each of the probe subsets comprises (a) a set of target probes, wherein the target probe set comprises one or more subsets of target probes, wherein each target probe subset comprises a plurality of pairs of target probes that specifically hybridize to a target nucleic acid; (b) a set of pre-amplifiers, wherein the pre-amplifier set comprises one or more subsets of pre-amplifiers, wherein the one or more pre-amplifier subsets comprise a pre-amplifier specific for each of the target probe pairs in the one or more target probe subsets, wherein each pre-amplifier comprises binding sites for the pair of target probes of one of the target probe subsets and a plurality of binding sites for an amplifier; (c) a set of amplifiers, wherein the amplifier set comprises one or more subsets of amplifiers specific for each pre-amplifier subset, wherein each amplifier subset comprises a plurality of amplifiers, wherein the amplifiers of one of the amplifier subsets comprise a binding site for the pre-amplifiers of one of the pre-amplifier subsets and a plurality of binding sites for a label probe; and (d) a set of label probes, wherein the label probe set comprises one or more subsets of label probes, wherein each label probe subset is specific for one of the amplifier subsets, wherein each label probe subset comprises a plurality of label probes, wherein the label probes in each of the label probe subsets comprise a label and a binding site for the amplifiers of one of the amplifier subsets, wherein the labels in each label probe subset are distinguishable between the label probe subsets; wherein the one or more label probe subsets in each probe subset specific for a target nucleic acid comprise at least one label or a combination of labels that is different for each probe subset (see FIG. 3).

In the embodiment described above, the implementation of the target labeling is at the level of the SGC, as shown in FIG. 3. Each subset of corresponding target probes, pre-amplifiers, amplifiers and label probes corresponds to a particular “color” of SGC. In the case where a single label is being used for a particular target nucleic acid (for example, in the case of four distinct labels, 1000, 0100, 0010 or 0001), a subset corresponds to one of the distinct labels. In the case where a set has only one subset, it is understood that the set corresponds to the one subset.

In one embodiment, the set of target probes, pre-amplifiers, amplifiers and label probes each comprise two or more subsets. In another embodiment, the set of target probes, pre-amplifiers, amplifiers and label probes each comprise three or more subsets. In another embodiment, the set of target probes, pre-amplifiers, amplifiers and label probes each comprise four or more subsets. In one embodiment, the target probe binding sites for the two or more subsets are intermingled on the target nucleic acid (see FIG. 5, top panel). As described in more detail below, this embodiment can be used to reduce miscoding.

The digital multiplexing scheme can also be implemented at the component level within an SGC (i.e., sub-SGC level). Such a multiplexing assay system comprises N different, label specific LPs and 2^{N}−1 unique, target specific SGCs. Each LP has a segment designed to hybridize to a label probe anchor (LA) on an AP molecule (i.e., the LA being the binding site on the amplifier for the label probe) in the SGC. A mixture of different LAs are designed and made to bind a set of pre-determined, different LPs, generating a unique combination of detectable signals that are used to identify the target. The sub-SGC level implementation is advantageous when the target sequence is very short. In addition, the probability of miscoding is reduced, which is described below. This approach can be adopted to detect a single base variant or a unique junction in the target sequence (see, for example, WO 2017/066211). Its disadvantage is that a relatively large number of unique SGCs (2^{N}−1) need to be developed.

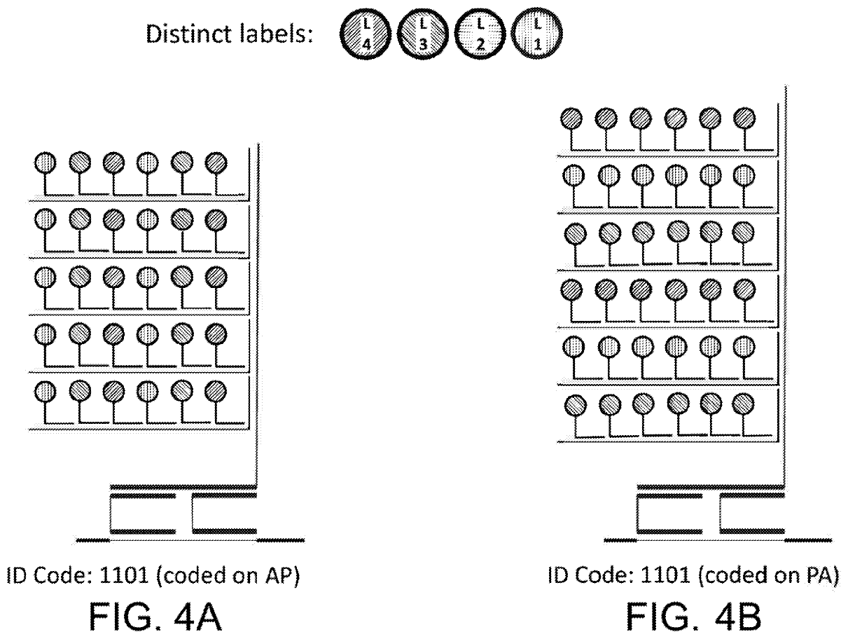

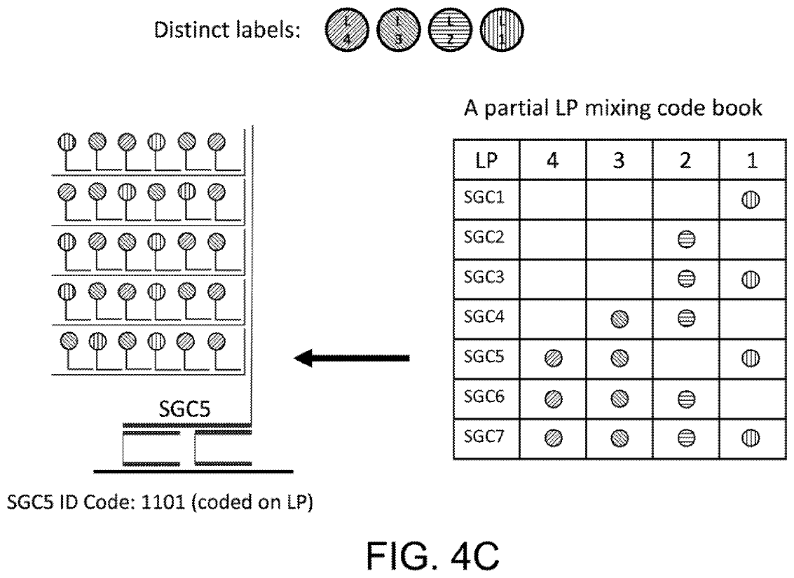

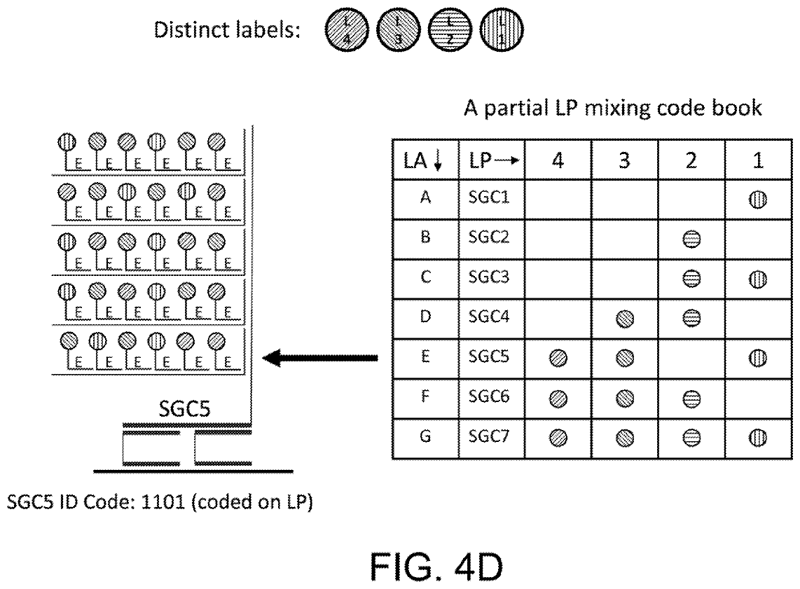

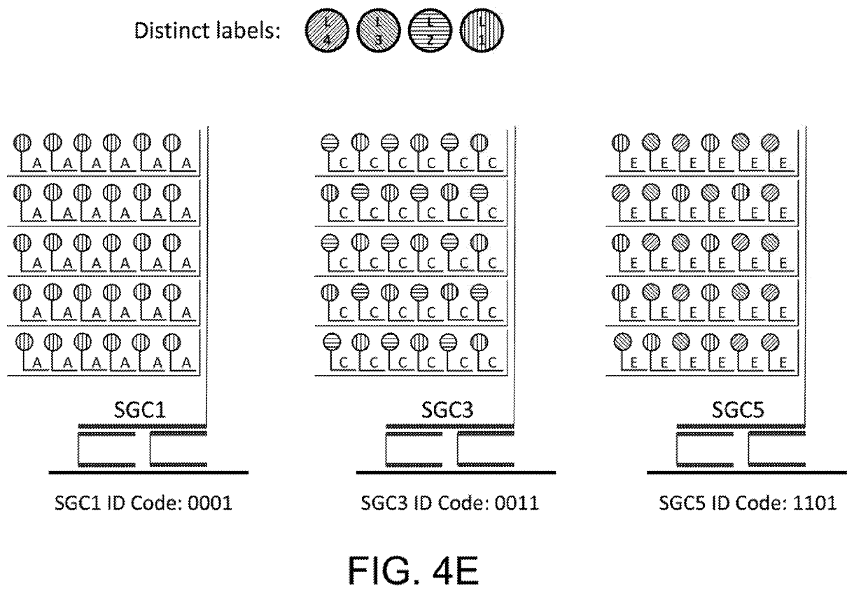

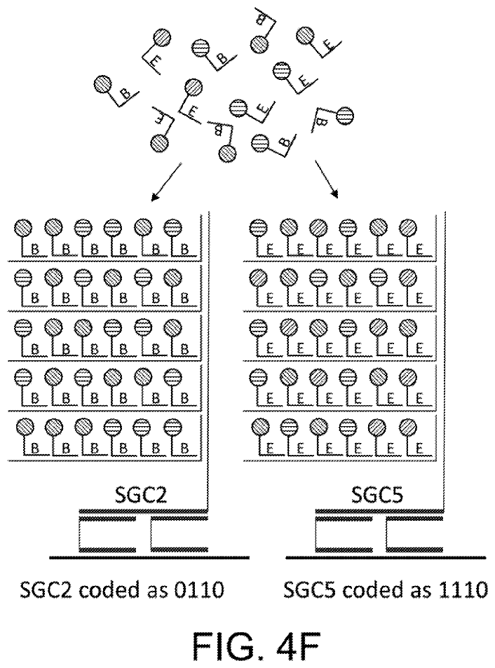

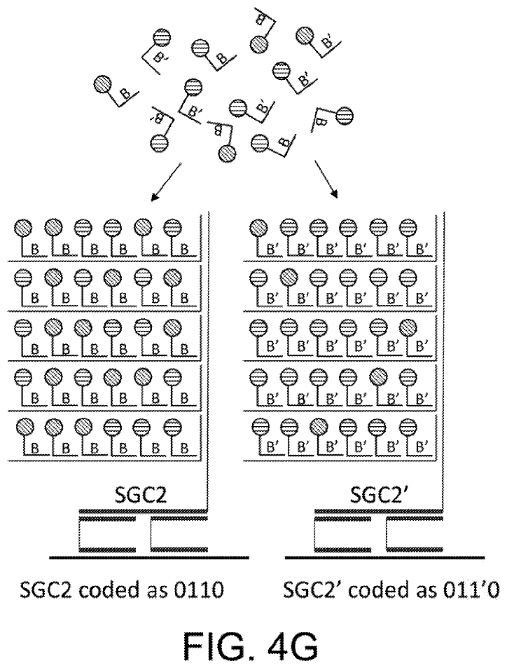

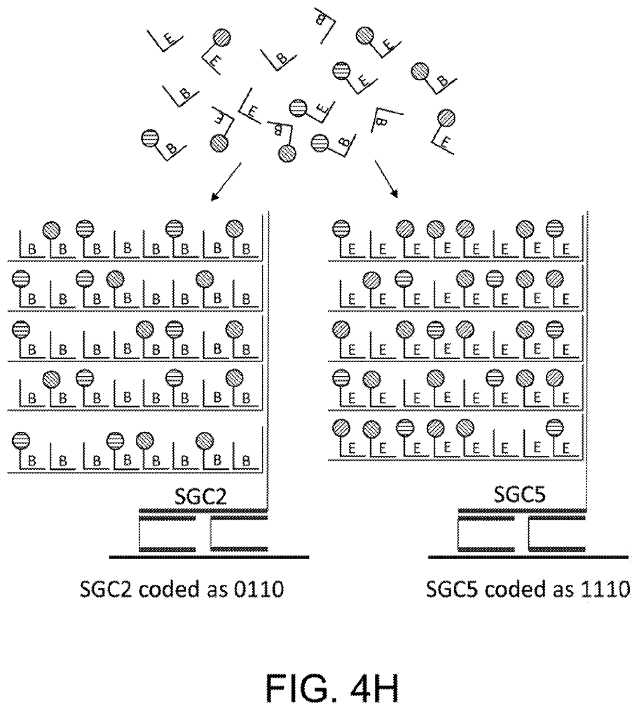

FIG. 4A illustrates one embodiment of sub-SGC implementation, where the above mentioned SGC ID code is implemented on the AP molecule. As shown in FIG. 4A, an AP has one region designed to bind to the amplifier anchor (AA) on the PA molecule (i.e., the AA being the binding site on the pre-amplifier for the amplifier) and another region comprising multiple segments of LAs (i.e., the binding sites on the amplifier for the label probes). In previously described methods, the amplifier used repeats of the same LA, that is, the amplifier had a plurality of binding sites for the same label probe. An embodiment of the invention using repeats of the same LA for binding a plurality of the same LPs to the amplifier is shown in FIG. 3. In the embodiment shown in FIG. 4A, however, a mixture of different LAs are designed according to the unique identification code of the SGC. For example, if the ID code of the SGC is L4, L3, L2, L1=1110, then equal number of LAs for LP type 4, 3, 2 are made on the AP molecule, which will bind a designed number of desired LP to generate the ID code in the assay. In the exemplary embodiment shown in FIG. 4A, the code for the SGC shown is 1101 using labels 4, 3 and 1.

In one embodiment, the invention provides a method for multiplex detection of a plurality of target nucleic acids in a cell, comprising (A) contacting a sample comprising a cell comprising a plurality of target nucleic acids with a set of probes, wherein the set of probes comprises subsets of probes comprising a plurality detectable labels that provide unique labeling of each target nucleic acid, wherein each probe subset comprises one or more distinct labels, wherein the number and/or combination of distinct labels is unique for each target nucleic acid; and (B) detecting the detectable labels bound to the respective target nucleic acids; and wherein each probe in each of the probe subsets comprises (a) a set of target probes, wherein the target probe set comprises a plurality of pairs of target probes that specifically hybridize to a target nucleic acid; (b) a set of pre-amplifiers, wherein the pre-amplifier set comprises a plurality of pre-amplifiers, wherein the pre-amplifiers comprise binding sites for the pairs of target probes and a plurality of binding sites for an amplifier; (c) a set of amplifiers, wherein the amplifier set comprises a plurality of amplifiers, wherein the amplifiers comprise a binding site for the pre-amplifiers and a plurality of binding sites for a label probe or two or more distinct label probes; and (d) a set of label probes, wherein the label probe set comprises a label probe or two or more distinct label probes, wherein each label probe comprises a label and a binding site for the amplifiers, wherein the labels in each distinct label probe are distinguishable between the distinct label probes; wherein the amplifier in each probe subset specific for a target nucleic acid comprises a binding site for a label or a combination of two or more distinct labels that is different for each probe subset (see FIG. 4A).

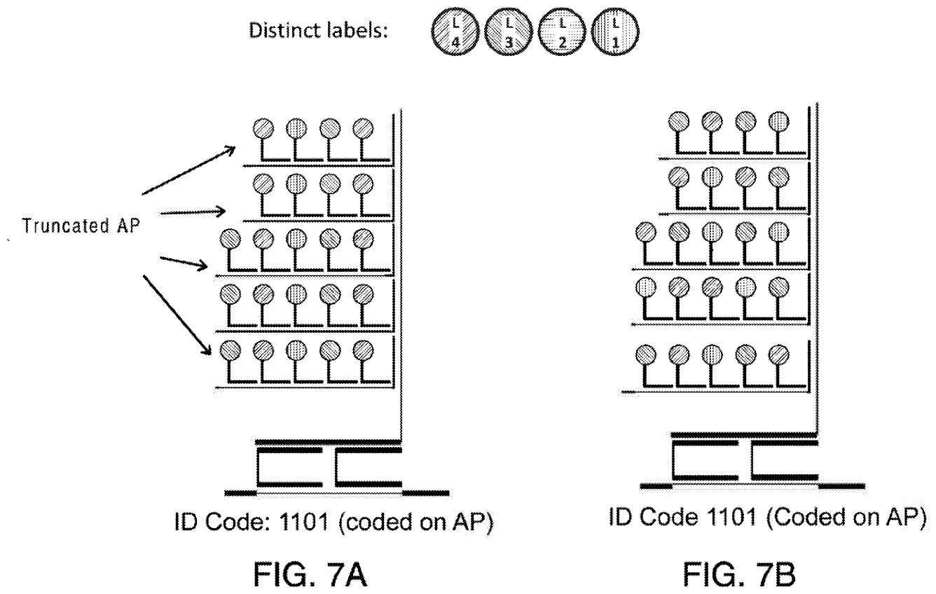

In one embodiment of such a method, the label probe set comprises two or more distinct label probes, wherein the amplifier set comprises a plurality of non-identical amplifiers, and wherein the binding sites for the two or more distinct label probes on each non-identical amplifier are in a different order on each non-identical amplifier (see FIG. 7B). This embodiment can be used to reduce miscoding, as described below in more detail.

FIG. 4B illustrates another embodiment of sub-SGC implementation, where the SGC ID code is implemented on the PA molecule. As shown in FIG. 4B, N “pure” AP molecules are made, each carrying the same LA for the same type of LP. A PA molecule has one region designed to bind to the SA (the signal anchor segment, i.e., the segment of the TP that binds to the pre-amplifier; see FIG. 1A) of a TP set and another region comprising multiple segments of AAs (amplifier anchors, i.e., the segments on the pre-amplifier that bind to the amplifiers). In previously disclosed methods, these are repeats of the same AA. In the embodiment shown in FIG. 1B, however, a mixture of different AAs are designed according to the unique identification code of the SGC. For example, if the ID code of the SGC is L4, L3, L2, L1=1010, then equal number of AAs for APs carrying LP type 4, 2 are made on the PA molecule, which will bind a designed number of desired LPs to generate the ID code in the assay. In the exemplary embodiment shown in FIG. 4B, the code for the SGC shown is 1101 using labels 4, 3 and 1.

In one embodiment, the invention provides a method for multiplex detection of a plurality of target nucleic acids in a cell, comprising (A) contacting a sample comprising a cell comprising a plurality of target nucleic acids with a set of probes, wherein the set of probes comprises subsets of probes comprising a plurality detectable labels that provide unique labeling of each target nucleic acid, wherein each probe subset comprises one or more distinct labels, wherein the number and/or combination of distinct labels is unique for each target nucleic acid; and (B) detecting the detectable labels bound to the respective target nucleic acids; and wherein each probe in each of the probe subsets comprises (a) a set of target probes, wherein the target probe set comprises a plurality of pairs of target probes that specifically hybridize to a target nucleic acid; (b) a set of pre-amplifiers, wherein the pre-amplifier set comprises a plurality of pre-amplifiers, wherein the pre-amplifiers comprise binding sites for the pairs of target probes and a plurality of binding sites for amplifiers; (c) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for the pre-amplifiers and a plurality of binding sites for a distinct label probe; and (d) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes; wherein the pre-amplifier in each probe subset specific for a target nucleic acid comprises a plurality of binding sites for the amplifier comprising a binding site for the label probe or a plurality of binding sites for the two or more distinct amplifiers comprising binding sites for the two or more distinct label probes, and wherein the label of the label probe or combination of two or more distinct labels of the two or more distinct label probes is different for each probe subset (see FIG. 4B).

In one embodiment of such a method, the plurality of amplifiers comprise two or more distinct amplifiers, and wherein the binding sites on the pre-amplifier for the distinct amplifiers are intermingled (see FIG. 6B). As described below in more detail, this embodiment can be used to reduce miscoding.

FIG. 4C illustrates yet another embodiment of sub-SGC implementation, where the SGC ID code is implemented on the LP molecule. In previously described methods, the LP sequences bound to a single SGC all carry the same label. The embodiment of FIG. 3 also utilizes the same labels on a given SGC. In the embodiment depicted in FIG. 4C, LP molecules binding to the same SGC can be a mixture of LPs each conjugated to a different label according to a predefined code book. For example, as shown in FIG. 4C, SGC5 LPs are a mixture of LPs conjugated to three different labels, creating the ID code 1011. The advantage of this embodiment is that the “coloring” of the SGC complex by the LPs will be completely randomized, which can further help to reduce coding errors. Since the SGC ID codes are not hard coded in the SGCs, this scheme provides the flexibility to assign different ID codes to different SGCs in different assay configurations simply by devising a different code book on the fly. In addition, the mixing of LPs can be made in unequal amounts to normalize the labeling intensities across the N labels, which again can help in reducing encoding/decoding errors. Furthermore, the mixing of LPs can be made according to predefined ratios of different labels such that each label can encode >1 bit of information. For example, a 1010 ID code could be distinguished from a 101′0 ID code where the 1′ refers to a specific label being present at a higher or lower concentration (and therefore providing a different relative signal intensity) than a 1. Each color can be provided at up to M relative concentrations such as 1, 1′, 1″, 1′″, etc., which will be limited by the number of distinct levels that can be reliably detected by the signal detection system and the number of available LA sites in each SGC (the higher the total number of LA sites, the larger the number of distinct levels can be detected). The disadvantage of this embodiment is that M^{N}−1 LP and LA sequences will be required to uniquely encode and decode each SGC. In comparison, in the embodiment shown in FIG. 4A, only N unique LPs and LAs are required.

As shown in FIG. 4C, the SGC ID code is implemented on the LP molecule. In this embodiment, LP molecules binding to the same SGC can be mixtures of LPs each conjugated to a different label according to a predefined code book. For example, as shown in FIG. 4C, SGC5 LPs are a mixture of LPs conjugated to three different labels, creating the ID code 1101, corresponding to labels 4, 3 and 1. The advantage of this embodiment is that the “coloring” of the SGC complex by the LPs will be completely randomized, which can further help to reduce coding errors. A partial LP mixing code book is shown on the right of FIG. 4C, with 7 different exemplary SGC codes shown using 4 labels.

FIG. 4D shows an embodiment of FIG. 4C in more detail. As depicted in FIG. 4D, for each SGC, a specific label anchor (LA, the binding site on the amplifier for the label probe) is assigned so that each SGC for a particular target nucleic acid has a plurality of the same LAs on the amplifier. The level at which combinatorial labeling can be provided is with the label probes (LPs). In this case, SGC5 is illustrated showing that the amplifiers comprise a plurality of identical LAs, labeled “E.” As shown in FIG. 4D, SGC5 is coded with 3 ID codes (1101) corresponding to 3 distinct label probes (4, 3 and 1), all of which have the same binding site for the plurality of “E” LAs on the corresponding amplifiers. Therefore, all three label probes (4, 3 and 1) are bound to the amplifiers of SGC5, thereby labeling the SGC5 target nucleic acid with the label code 1101.

FIG. 4E shows an embodiment of FIG. 4C in more detail. The SGC5 of FIG. 4D is shown bound to its respective target nucleic acid, with the label probes having an “E” binding site bound to the respective “E” LAs of the SGC5 amplifiers (as in FIG. 4D). Also shown are two additional exemplary SGCs bound to their respective target nucleic acids. SGC1, coded as shown in FIG. 4D, comprises a plurality of identical LAs, labeled “A.” SGC1 is coded with 1 ID code (0001) corresponding to a label probe (1), which has the binding site for the plurality of “A” LAs of the SGC1 amplifiers. Therefore, the label probe “1” is bound to the amplifiers of SGC1, thereby labeling the SGC1 target nucleic acid with the label code 0001. SGC3, coded as shown in FIG. 4D, comprises a plurality of identical LAs, labeled “C.” SGC3 is coded with 2 ID probes (0011) corresponding to 2 distinct label probes (2 and 1), both of which have the same binding site for the plurality of “C” LAs on the corresponding SGC3 amplifiers. Therefore, both label probes (2 and 1) are bound to the amplifiers of SGC3, thereby labeling the SGC3 target nucleic acid with the label code 0011.

FIG. 4F shows an embodiment of FIG. 4C in more detail. FIG. 4F illustrates that, once an SGC for a particular target nucleic acid has been designed, the actual coding for the target nucleic acid can be readily modified simply by changing the labels on the label probes that bind to the amplifiers of a particular SGC. For example, in FIG. 4D, SGC2 comprises amplifiers with “B” LAs and is coded as 0010 using label 2. In FIG. 4F, the same SGC assembly can be used with respect to the target probes, pre-amplifier, and amplifiers with “B” LAs, but instead of using “B” LA-binding label probes with only label 2 as in FIG. 4B, “B” LA-binding label probes can be used that have a mixture of labels 3 and 2 such that SGC2 is now coded with both labels (0110). Thus, labels 3 and 2 (0110) are bound to “B” LAs on the SGC2 amplifiers. Similarly, SGC5 comprising amplifiers with “E” LAs is now coded in FIG. 4F as 1110 by using label probes with “E” LA-binding label probes that have a mixture of labels 4, 3 and 2 (1110), instead of labels 4, 3 and 1 (1101) as shown in FIG. 4D.

FIG. 4G shows an embodiment of FIG. 4C in more detail. In FIG. 4G, an additional “coding” can be implemented by using different ratios of label probes. As shown in FIG. 4G, rather than binding the distinct label probes in equivalent amounts, the ratio of distinct label probes bound to the corresponding amplifiers can be varied such that not only the presence of a particular label but also the relative amount of a particular label can be used as another way to provide a distinct label. As shown in FIG. 4G, SGC2 is coded as 0110 with labels 3 and 2, whereas SGC2′ can be used to code a different target nucleic acid using the code 011′0 and the same labels 3 and 2, but where the ratio of label 2 to label 3 bound to SGC2′ is different than the ratio of label 2 to label 3 bound to SGC2. The two target nucleic acids are labeled with the same label probes, but the target nucleic acids can be distinguished based on the relative amounts of the two label probes bound to the respective target nucleic acids.

As described herein, an additional “coding” can be implemented by using different ratios of label probes. Rather than adding the distinct label probes in equivalent amounts, the ratio of distinct label probes can be varied such that not only the presence of a particular label but also the relative amount of a particular label can be used as another way to provide a distinct label, as described herein. One way to achieve different ratios of labeling is by providing different relative proportions of labels that bind to the same LA. For example, in one experiment the proportion of the two distinct label probes provided to an SGC for binding to one target nucleic acid can be at a 1:1 ratio. In a separate experiment, the same label probes can be used to label a different target nucleic acid with a different ratio (for example, 2:1) of the two distinct probes. In this case, the two target nucleic acids are labeled with the same label probes, but the target nucleic acids can be distinguished based on the relative amounts of the two label probes bound to the respective target nucleic acids.

A similar type of labeling two target nucleic acids with different ratios of the same label probes can be achieved for detection concurrently, for example, by using two sets of label probes for detection of two target nucleic acids, where the SGC for each target contains different LAs on the respective amplifiers. In this case, the label probe sets with the corresponding amplifier binding sites are contacted with the cell such that the same label in the respective sets are in different ratios between the two sets. In this case, the two target nucleic acids are labeled with the same label probes, but the target nucleic acids can be distinguished based on the relative amounts of the two label probes bound to the respective target nucleic acids.

Another way to utilize different ratios of labels to detect different target nucleic acids can be implemented in the embodiments depicted in FIGS. 4A and 4B. For example, in an embodiment similar to FIG. 4A, rather than including equivalent numbers of LAs for each of the respective label probes, the LAs for each distinct LP can be incorporated into the amplifier to provide a specified ratio between different LPs, thereby labeling a target nucleic acid with one ratio. A different ratio of LAs specific for the same LPs can be utilized to label a different target nucleic acid. A similar approach can be utilized in an embodiment depicted in FIG. 4B, where the ratio of binding sites for the amplifiers (AAs) on the pre-amplifier are included in different ratios on the SGC for one target nucleic acid compared to the SGC for another target nucleic acid. These embodiments provide for additional “codes” based on the combinations of labels and the ratios of distinct labels, where additional “codes” can be implemented with the same labels.

As described herein, in some embodiments, an SGC for different target nucleic acids will have a different number of labels in the code (e.g., 1000, 1100, 1110 and 1111) (see FIGS. 4C and 4D). In this situation and in the case where the number of LAs on the respective SGCs is the same, if the number of label probes are added to the SGCs, the SGC coded 1000 will have a higher number of bound labels (label 4 probes) than the number of label 4 probes bound to an SGC coded 1111, since label probe 4 can bind to all of the sites on one of the SGCs but on ¼ of the sites on the other SGC. In some embodiments of the invention, it can be desirable to normalize the amount of label bound to different SGCs coded by different numbers of distinct labels. In an exemplary embodiment as shown in FIG. 4H, two target nucleic acids are shown with two bound SGCs, SGC2 and SGC5. SGC2 is coded as 0110 with labels 3 and 2, and SGC5 is coded as 1110 with labels 4, 3 and 2. In this case, where all of the label probes bind to the respective LAs, “B” LAs in the case of SGC2 and “E” LAs in the case of SGC5, and assuming that the SGC2 and SGC5 have approximately the same number of LAs in the respective SGCs, the number of respective labels that can bind to SGC2 will be higher than the number of respective labels that bind to SGC5 (i.e., the 2 distinct labels for SGC2 (labels 3 and 2) and the 3 distinct labels for SGC5 (labels 4, 3 and 2) will be bound to the same number of sites, resulting in a higher number of labels 3 and 2 being bound to SGC2 than SGC5 since some of the SGC5 sites are occupied by label 4). If desired, the number of labels (and therefore intensity of signal) can be normalized by including “blank” label probes, i.e., probes having a binding site for the respective LAs (in this case “B” for SGC2 and “E” for SGC5) but without a label. For example, if it is desired to compare SGC2 and SGC5 with equal intensity signals for the respective labels, ⅓ “blank” label probes can be included with the mixture of “B” LA-specific probes so that the intensity of labels 3 and 2 will be the same on both SGCs (i.e., ⅓ of SGC2 occupied by “blank” label probes and ⅓ of SGC5 occupied by label 4). In another example, if a multiplex assay is being performed where some SGCs include 4 labels, then the assay can be performed so that the same proportion of “blank” label probes are included in the label probe sets using less than 4 labels, for example, ½ “blank” label probes can be included with the SG2-specific label probes coded by 2 distinct labels (0110) and ¼ “blank” label probes can be included with the SGC5-specific label probes coded by 3 distinct labels (1110) so that the amount of each distinct label probe, 4, 3, 2 and 1, bound to the respective SGCs is the same on each SGC. Using such “blank” label probes can also be utilized in combination with distinct label probes to provide for a desired proportion of respective labels, such as a desired ratio of label probes on an SGC.

In one embodiment, the invention provides a method for multiplex detection of a plurality of target nucleic acids in a cell, comprising (A) contacting a sample comprising a cell comprising a plurality of target nucleic acids with a set of probes, wherein the set of probes comprises subsets of probes comprising a plurality detectable labels that provide unique labeling of each target nucleic acid, wherein each probe subset comprises one or more distinct labels, wherein the number and/or combination of distinct labels is unique for each target nucleic acid; and (B) detecting the detectable labels bound to the respective target nucleic acids; and wherein each probe in each of the probe subsets comprises (a) a set of target probes, wherein the target probe set comprises a plurality of pairs of target probes that specifically hybridize to a target nucleic acid; (b) a set of pre-amplifiers, wherein the pre-amplifier set comprises a plurality of pre-amplifiers, wherein the pre-amplifiers comprise binding sites for the pairs of target probes and a plurality of binding sites for an amplifier; (c) a set of amplifiers, wherein the amplifier set comprises a plurality of amplifiers, wherein the amplifiers comprise a binding site for the pre-amplifiers and a plurality of identical binding sites for a label probe; and (d) a set of label probes, wherein the label probe set comprises a label probe or two or more distinct label probes, wherein each label probe comprises a label and a binding site for the amplifiers, wherein the binding site for the amplifier is the same for each label probe, wherein the labels in each distinct label probe are distinguishable between the distinct label probes; wherein the amplifier in each probe subset specific for a target nucleic acid comprises a binding site for a label or a combination of two or more distinct labels that is different for each probe subset (see FIG. 4C).

In one embodiment of the method, the distinct labels of the two or more distinct label probes are the same in two probe subsets for two target nucleic acids and wherein the ratio of label probes in one probe subset is different than the ratio of label probes in the second probe subset, wherein a difference in ratios of distinct label probes in the first probe subset and the second probe subset distinguish the two target nucleic acids (see FIG. 4G).

Similar approaches can be implemented on other components of the SGC and the two embodiments shown in FIG. 4A and FIG. 4B can be used in combination to ensure the pre-determined mixture of LPs can be assembled onto the SGC to generate the ID code.

In the embodiments described herein, the label on each LP can be an indirect label, such as enzymes (e.g., horseradish peroxidase (HRP)) or haptens (e.g., digoxygenin) that can be detected at a later step, as well as additional labels as disclosed herein. An example of embodiment is the use of HRP as the label in the LP, which can provide for additional signal amplification by using a fluorescent dye conjugated to tyramide, as described below in more detail. Enhanced signals can be advantageous for both signal detection and reducing coding errors in sub-optimal samples such as formalin-fixed paraffin-embedded tissues or tissues with significant autofluorescence.

In some embodiments of the invention, the methods of the invention can be modified to reduce miscoding. Miscoding can occur when the signal from a type of LP designed to be present with a particular target is undetectable (i.e., errant “0”), or background noise is misinterpreted as a signal from a particular LP (i.e., errant “1”). Errant “1” type miscoding can be largely eliminated by setting up an appropriate threshold. Signal level from the area surrounding the image dot as well as the global background level can be used to reference the background. Methods for reducing Errant “0” type miscoding are described herein.

When the SGC level code implementation method described previously is used, errant “0” type miscoding can occur when SGCs of a certain LP type are not attached to the target due to limited TP accessibility to the target or target degradation. It is therefore important that each label-specific SGC has many copies in the set designed to bind to the same target so that there are statistically many chances for each needed LPs to be present in the detected signal. This means that the target sequence has to be very long in order to reduce the miscoding rate. For example, assuming four different LPs are used, 20 copies of each LP specific SGCs can be needed to ensure reliable detection and each SGC binds to a TP set occupying 50 nucleotides (50nt), so the total target length in this case has to be 4×20×50=4000 nucleotides. Also, it is advantageous to intertwine different SGCs along the target, as shown in FIG. 5. If different SGC types are positioned apart in separate groups, as depicted in FIG. 5, lower panel, a certain section of the target may be blocked or masked, thereby preventing attachment of one specific SGC type, which will result in miscoding. Designing the target probes in the SGC of the same type, when more than one type of SGC is used, to bind to the target nucleic acid at sites that are intermingled or intertwined, as shown in FIG. 5, upper panel, reduces the chance of miscoding.

From the point of view of reducing miscoding, there are advantages to implementing the ID code at the sub-SGC level because, once a TP set is successfully hybridized to the target, there are many more chances for different LPs to successfully attach to the SGC without bias. Arranging different labels into alternating positions is still a very important strategy to reduce the chance of miscoding. As shown in FIG. 6A, this particular SGC is miscoded from “1001” to “0001” because the PA is truncated, which can occur during manufacturing of the PA. The same truncation, however, will not cause the miscoding if the labels are intertwined or intermingled on the PA, if the embodiment shown in FIG. 4B is implemented as shown in FIG. 6B. A similar strategy can be used in a configuration with AP level coding, as illustrated in FIG. 4A. An additional method to minimize the potential miscoding caused by truncation is to randomize the position of different labels on the AP or PA that encodes the target specific code, as illustrated in FIG. 7, in which the multiplexing channel ID is encoded on the AP molecule. In FIG. 7A, different label probes are positioned on each AP in exactly the same way, that is, each amplifier is the same. Truncation of the AP, for example, during manufacturing, may cause substantial reduction in certain label compared to others. This imbalance increases the chance of miscoding. In the most severe case, truncation may cause the loss of all copies of one certain label leading to an outright miscode. In FIG. 7B, locations of different labels on the AP are purposefully randomized. Truncation therefore does not cause a large bias in the numbers and types of labels in the SGC. The APs are provided as a plurality of amplifiers, where a mix of non-identical amplifiers is included, where the position of LAs for specific label probes are distributed differently and can be randomized on the non-identical amplifiers. Randomization of different labels in the SGCs can be achieved by using one or combinations of the embodiments described herein.

When an errant “0” type miscoding occurs, a target with more “1”s in its ID code is mis-identified as another target with fewer “1”s in the ID code. In most situations, the probability of miscoding is low (e.g., <5%). When the quantities of targets are in a similar level, such miscoding does not significantly impact the results. Miscoding can have a significant impact if one target is present at a significantly higher quantity than the other target that it miscoded into (i.e., one target is miscoded to be misread as another target due to differences in amounts of the two targets). Therefore, one important method to reduce the impact of miscoding is to assign ID codes with fewer “1”s to higher quantity targets if the relative quantities of the targets are known. For example, the multiplex channels of T1, T2, T4 and T8 in Table 1 are each coded by a single label. These codes can be reserved for targets with the highest quantities because if any errant “0” miscoding occurs in these channels, they will not be mis-interpreted into the signals from other multiplex channels.

Many error detection schemes used in the digital communication field can be adopted to detect miscoding. For example, a parity check can ensure accurate data transmission during communication. A parity bit is appended to the original data bits to make an even or odd number of total data bits. For example, the signal from one of the N Labels can be used as a “parity check” bit (L1 in the example codebook in Table 2). The bit will be made “1” (present) or “0” (absent) to make the total number of “1”s in the N label system odd (i.e., odd parity check) or even (i.e., even parity check). Depending on the result of the parity check, the detected target may or may not be counted. This parity check scheme can detect single or odd number of bit errors but cannot detect double or even number bit errors. This can substantially reduce the probability of miscoding. For example, if the chance of single bit error is 5%, the chance of double bit error is theoretically 0.25%. The price for using such a parity check is that the number of multiplexing channels is reduced to 2^{N-1}−1 or 2^{N-1} if an even or odd parity check scheme is adopted, respectively (e.g., 7 or 8 SGC codes in Table 2 compared to 15 SGC codes in Table 1). A code book can be designed to include an odd (or even) number of labels. In the example shown in Table 1, for an odd number, the allowed channels from Table 1 would be T1, T2, T4, T7, T8, T11, T13, and T14. If one of the other channels is detected, then it must be an error.

TABLE 2
Codebook with Parity Check.

| | | Presence of labels | | | | |
|---|---|---|---|---|---|---|
| | | (“1” present, “0” absent) | | | | |
| | | | | | L1 = Even | L1 = Odd |
| | | L4 | L3 | L2 | Parity Bit | Parity Bit |
| ID Codes of | T1 | 0 | 0 | 1 | 1 | 0 |
| Targets or | T2 | 0 | 1 | 0 | 1 | 0 |
| Multiplexing | T3 | 1 | 0 | 0 | 1 | 0 |
| Channels | T4 | 0 | 1 | 1 | 0 | 1 |
| | T5 | 1 | 0 | 1 | 0 | 1 |
| | T6 | 1 | 1 | 0 | 0 | 1 |
| | T7 | 1 | 1 | 1 | 1 | 0 |
| | T8 | 0 | 0 | 0 | | 1 |

As described herein, a specific target nucleic acid can be labeled with more than one distinct label. In this case, a single “dot” will be comprised of two or more distinct labels. A dot comprising more than one label can be deconvoluted to identify the individual labels in the dot using well known methods. Such well known methods include the Richardson-Lucy deconvolution algorithm (see Example I) as described previously (Biggs et al., Applied Optics, Vol. 36, No. 8, (1997); Hanisch et al., “Deconvolutions of Hubble Space Telescope Images and Spectra, Deconvolution of Images and Spectra,” Ed. P. A. Jansson, 2nd ed., Academic Press CA, (1997)). Other methods for deconvolution include, but are not limited to, Wiener deconvolution, regularized filter deconvolution, and the like (Gonzalez et al., “Digital Image Processing,” Addison-Wesley Publishing Company, Inc. (1992)).

The embodiments described above and depicted in FIG. 3-7 show SGCs with pre-amplifiers, amplifiers and label probes. It is understood that the same principles can be applied to an SGC where a pre-pre-amplifier component is included in the SGC, as disclosed herein (see, for example, FIGS. 5B, 5C, 6B and 6C for examples of SGCs with a pre-pre-amplifier layer).

In one embodiment, the invention provides a method for multiplex detection of a plurality of target nucleic acids in a cell, comprising (A) contacting a sample comprising a cell comprising a plurality of target nucleic acids with a set of probes, wherein the set of probes comprises subsets of probes comprising a plurality detectable labels that provide unique labeling of each target nucleic acid, wherein each probe subset comprises one or more distinct labels, wherein the number and/or combination of distinct labels is unique for each target nucleic acid; and (B) detecting the detectable labels bound to the respective target nucleic acids; and wherein each probe in each of the probe subsets comprises (a) a set of target probes, wherein the target probe set comprises one or more subsets of target probes, wherein each target probe subset comprises a plurality of pairs of target probes that specifically hybridize to a target nucleic acid; (b) a set of pre-pre-amplifiers, wherein the pre-pre-amplifier set comprises one or more subsets of pre-pre-amplifiers, wherein the one or more pre-pre-amplifier subsets comprise a pre-pre-amplifier specific for each of the target probe pairs in the one or more target probe subsets, wherein each pre-pre-amplifier comprises binding sites for the pair of target probes of one of the target probe subsets and a plurality of binding sites for a pre-amplifier; (c) a set of pre-amplifiers, wherein the pre-amplifier set comprises one or more subsets of pre-amplifiers, wherein the one or more pre-amplifier subsets comprise a pre-amplifier specific for the pre-pre-amplifiers in the one or more pre-pre-amplifier subsets, wherein each pre-amplifier comprises binding sites for the pre-pre-amplifiers of one of the pre-pre-amplifier subsets and a plurality of binding sites for an amplifier; (d) a set of amplifiers, wherein the amplifier set comprises one or more subsets of amplifiers specific for each pre-amplifier subset, wherein each amplifier subset comprises a plurality of amplifiers, wherein the amplifiers of one of the amplifier subsets comprise a binding site for the pre-amplifiers of one of the pre-amplifier subsets and a plurality of binding sites for a label probe; and (e) a set of label probes, wherein the label probe set comprises one or more subsets of label probes, wherein each label probe subset is specific for one of the amplifier subsets, wherein each label probe subset comprises a plurality of label probes, wherein the label probes in each of the label probe subsets comprise a label and a binding site for the amplifiers of one of the amplifier subsets, wherein the labels in each label probe subset are distinguishable between the label probe subsets; wherein the one or more label probe subsets in each probe subset specific for a target nucleic acid comprise at least one label or a combination of labels that is different for each probe subset. This embodiment is similar to FIG. 3 except that a pre-pre-amplifier is included in the SGC.

In one embodiment, the set of target probes, pre-amplifiers, amplifiers and label probes each comprise two or more subsets. In another embodiment of such a method, the set of target probes, pre-amplifiers, amplifiers and label probes each comprise three or more subsets. In another embodiment, the set of target probes, pre-amplifiers, amplifiers and label probes each comprise four or more subsets.

In one embodiment, the target probe binding sites for the two or more subsets are intermingled on the target nucleic acid (see FIG. 5, top panel, with a pre-pre-amplifier included in the SGC).

In one embodiment, the invention provides a method for multiplex detection of a plurality of target nucleic acids in a cell, comprising (A) contacting a sample comprising a cell comprising a plurality of target nucleic acids with a set of probes, wherein the set of probes comprises subsets of probes comprising a plurality detectable labels that provide unique labeling of each target nucleic acid, wherein each probe subset comprises one or more distinct labels, wherein the number and/or combination of distinct labels is unique for each target nucleic acid; and (B) detecting the detectable labels bound to the respective target nucleic acids; and wherein each probe in each of the probe subsets comprises (a) a set of target probes, wherein the target probe set comprises a plurality of pairs of target probes that specifically hybridize to a target nucleic acid; (b) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier; (c) a set of pre-amplifiers, wherein the pre-amplifier set comprises a plurality of pre-amplifiers, wherein the pre-amplifiers comprise binding sites for the pre-pre-amplifiers and a plurality of binding sites for an amplifier; (d) a set of amplifiers, wherein the amplifier set comprises a plurality of amplifiers, wherein the amplifiers comprise a binding site for the pre-amplifiers and a plurality of binding sites for a label probe or two or more distinct label probes; and (e) a set of label probes, wherein the label probe set comprises a label probe or two or more distinct label probes, wherein each label probe comprises a label and a binding site for the amplifiers, wherein the labels in each distinct label probe are distinguishable between the distinct label probes; wherein the amplifier in each probe subset specific for a target nucleic acid comprises a binding site for a label or a combination of two or more distinct labels that is different for each probe subset. This embodiment is similar to FIG. 4A except that a pre-pre-amplifier is included in the SGC.

In one embodiment, the label probe set comprises two or more distinct label probes, wherein the amplifier set comprises a plurality of non-identical amplifiers, and wherein the binding sites for the two or more distinct label probes on each non-identical amplifier are in a different order on each non-identical amplifier (similar to FIG. 7B except with a pre-pre-amplifier in the SGC).

In one embodiment, the invention provides a method for multiplex detection of a plurality of target nucleic acids in a cell, comprising (A) contacting a sample comprising a cell comprising a plurality of target nucleic acids with a set of probes, wherein the set of probes comprises subsets of probes comprising a plurality detectable labels that provide unique labeling of each target nucleic acid, wherein each probe subset comprises one or more distinct labels, wherein the number and/or combination of distinct labels is unique for each target nucleic acid; and (B) detecting the detectable labels bound to the respective target nucleic acids; and wherein each probe in each of the probe subsets comprises (a) a set of target probes, wherein the target probe set comprises a plurality of pairs of target probes that specifically hybridize to a target nucleic acid; (b) a set of pre-pre-amplifiers, wherein the pre-pre-amplifier set comprises a plurality of pre-pre-amplifiers, wherein each pre-pre-amplifier comprises binding sites for the pairs of target probes and a plurality of binding sites for a pre-amplifier; (c) a set of pre-amplifiers, wherein the pre-amplifier set comprises a plurality of pre-amplifiers, wherein the pre-amplifiers comprise binding sites for the pre-pre-amplifiers and a plurality of binding sites for amplifiers; (d) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for the pre-amplifiers and a plurality of binding sites for a distinct label probe; and (e) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes; wherein the pre-amplifier in each probe subset specific for a target nucleic acid comprises a plurality of binding sites for the amplifier comprising a binding site for the label probe or a plurality of binding sites for the two or more distinct amplifiers comprising binding sites for the two or more distinct label probes, and wherein the label of the label probe or combination of two or more distinct labels of the two or more distinct label probes is different for each probe subset. This embodiment is similar to FIG. 4B except that a pre-pre-amplifier is included in the SGC.

In one embodiment, the plurality of amplifiers comprise two or more distinct amplifiers, and wherein the binding sites on the pre-amplifier for the distinct amplifiers are intermingled (similar to FIG. 6B except with a pre-pre-amplifier layer in the SGC).

In one embodiment, the invention provides a method for multiplex detection of a plurality of target nucleic acids in a cell, comprising (A) contacting a sample comprising a cell comprising a plurality of target nucleic acids with a set of probes, wherein the set of probes comprises subsets of probes comprising a plurality detectable labels that provide unique labeling of each target nucleic acid, wherein each probe subset comprises one or more distinct labels, wherein the number and/or combination of distinct labels is unique for each target nucleic acid; and (B) detecting the detectable labels bound to the respective target nucleic acids; and wherein each probe in each of the probe subsets comprises (a) a set of target probes, wherein the target probe set comprises a plurality of pairs of target probes that specifically hybridize to a target nucleic acid; (b) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier or for two or more distinct pre-amplifiers; (c) a set of pre-amplifiers, wherein the pre-amplifier set comprises a plurality of pre-amplifiers, wherein the plurality of pre-amplifiers comprise a pre-amplifier comprising a binding site for the pre-pre-amplifiers and a plurality of binding sites for an amplifier, or wherein the plurality of pre-amplifiers comprise two or more distinct pre-amplifiers, wherein each distinct pre-amplifier comprises a binding site for the pre-pre-amplifiers and a plurality of binding sites for a distinct amplifier; (d) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for one of the distinct pre-amplifiers and a plurality of binding sites for a distinct label probe; and (e) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes; wherein the pre-pre-amplifier in each probe subset specific for a target nucleic acid comprises a plurality of binding sites for the pre-amplifier comprising a plurality of binding sites for the amplifier comprising a binding site for the label probe, or a plurality of binding sites for the two or more distinct pre-amplifiers each comprising a plurality of binding sites for one of the two or more distinct amplifiers comprising binding sites for one of the two or more distinct label probes, and wherein the label of the label probe or combination of two or more distinct labels of the two or more distinct label probes is different for each probe subset. This embodiment is similar to FIG. 4B, except that the combinatorial labeling is implemented at the level of one or more distinct pre-amplifiers binding to the pre-pre-amplifier, rather than at the level of one or more distinct amplifiers binding to the pre-amplifier, as shown in FIG. 4B.

In one embodiment, the plurality of pre-amplifiers comprise two or more distinct pre-amplifiers, and wherein the binding sites on the pre-pre-amplifier for the distinct pre-amplifiers are intermingled. This embodiment is similar to FIG. 6B except that the combinatorial labeling is implemented at the level of one ore more distinct pre-amplifiers binding to the pre-pre-amplifier, rather than at the level of one or more distinct amplifiers binding to the pre-amplifier, as shown in FIG. 6B.

In one embodiment, the invention provides a method for multiplex detection of a plurality of target nucleic acids in a cell, comprising (A) contacting a sample comprising a cell comprising a plurality of target nucleic acids with a set of probes, wherein the set of probes comprises subsets of probes comprising a plurality detectable labels that provide unique labeling of each target nucleic acid, wherein each probe subset comprises one or more distinct labels, wherein the number and/or combination of distinct labels is unique for each target nucleic acid; and (B) detecting the detectable labels bound to the respective target nucleic acids; and wherein each probe in each of the probe subsets comprises (a) a set of target probes, wherein the target probe set comprises a plurality of pairs of target probes that specifically hybridize to a target nucleic acid; (b) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier; (c) a set of pre-amplifiers, wherein the pre-amplifier set comprises a plurality of pre-amplifiers, wherein the pre-amplifiers comprise binding sites for the pairs of target probes and a plurality of binding sites for an amplifier; (d) a set of amplifiers, wherein the amplifier set comprises a plurality of amplifiers, wherein the amplifiers comprise a binding site for the pre-amplifiers and a plurality of identical binding sites for a label probe; and (e) a set of label probes, wherein the label probe set comprises a label probe or two or more distinct label probes, wherein each label probe comprises a label and a binding site for the amplifiers, wherein the binding site for the amplifier is the same for each label probe, wherein the labels in each distinct label probe are distinguishable between the distinct label probes; wherein the amplifier in each probe subset specific for a target nucleic acid comprises a binding site for a label probe or a combination of two or more distinct label probes that is different for each probe subset. This embodiment is similar to FIG. 4C except that a pre-pre-amplifier is included in the SGC.

In one embodiment of the method, the distinct labels of the two or more distinct label probes are the same in two probe subsets for two target nucleic acids and wherein the ratio of label probes in one probe subset is different than the ratio of label probes in the second probe subset, wherein a difference in ratios of distinct label probes in the first probe subset and the second probe subset distinguish the two target nucleic acids.

In general, when using distinct and distinguishable labels for multiplex detection of target nucleic acids, there is a limit to the number of distinct labels that can be distinguished concurrently. For example, in the case of fluorescent labels, in order for multiple labels to be detected simultaneously, there should be spectral separation of the multiple emissions from the fluorophores so that the fluorescence microscope can distinguish the fluorophores concurrently. The need for spectral separation of the emissions from the fluorophores limits the number of fluorophores that can be visualized simultaneously. The present invention circumvents this limitation by detecting labels iteratively, such that the same fluorophores can be used in sequential rounds to detect different target nucleic acids.

The orthogonal nature of detection systems that can be used in the methods of invention is depicted in FIG. 10. FIG. 10A shows one embodiment, with three exemplary target nucleic acids and the respective orthogonal detection systems, also referred to herein as signal generating complexes (SGCs). As depicted in FIG. 10A, each of the target nucleic acids is hybridized to specific target probe pairs (TP1a and TP1b, TP2a and TP2b, TP3a and TP3b), which in turn are hybridized to respective specific pre-amplifiers (PA1, PA2, PA3), which in turn are hybridized to a respective specific plurality of amplifiers (AMP1, AMP2, AMP3), which are in turn hybridized to a respective specific plurality of label probes (LP1, LP2, LP3). FIG. 10B shows another embodiment, with two exemplary target nucleic acids and the respective orthogonal detection systems. As depicted in FIG. 10B, each of the target nucleic acids is hybridized to specific target probe pairs (TP1a and TP1b, TP2a and TP2b), which in turn are hybridized to respective specific pre-pre-amplifiers (PPA1, PPA2), which in turn are hybridized to respective specific plurality of pre-amplifiers (PA1, PA2), which in turn are hybridized to a respective specific plurality of amplifiers (AMP1, AMP2), which are in turn hybridized to a respective specific plurality of label probes (LP1, LP2). FIG. 10C shows another embodiment, with two exemplary target nucleic acids and the respective orthogonal detection systems. As depicted in FIG. 10C, each of the target nucleic acids is hybridized to specific target probe pairs (TP1a and TP1b, TP2a and TP2b), which in turn are hybridized to respective specific pairs of pre-pre-amplifiers (PPA1a and PPA1b, PPA2a and PPA2b), which in turn are both hybridized to respective specific pre-amplifiers (PA1 and PA2), which in turn are hybridized to respective specific amplifiers (AMP1 and AMP2), which in turn are hybridized to respective specific label probes (LP1 and LP2). For simplicity, a plurality of amplifiers are depicted bound to one of the pre-amplifiers, but it is understood that the amplifiers can bind to each of the pre-amplifiers. As shown in FIG. 10, each nucleic acid target has a specific detection system, for which binding of the components are mediated by unique binding sites that provide for binding to one specific complex but not to another. Such unique binding sites for hybridization of components of an SGC to a specific target nucleic acid can be achieved designing binding sites (nucleic acid sequences) to provide the desired specificity, as well known in the art and described herein. This orthogonal detection system, where each target is uniquely labeled, allows the detection of multiple target nucleic acids in the same sample.

In some embodiments as described herein, the methods utilize orthogonal amplification systems to uniquely label target nucleic acids so that multiple target nucleic acids can be analyzed in the same sample and even in the same cell. The invention utilizes the building of signal generating complexes (SGCs) that are specific for particular target nucleic acids so that each target nucleic acid can be uniquely identified. In one embodiment, a sample is contacted with target probe sets comprising a pair of target probes that can specifically hybridize to a target nucleic acid. The sample is also contacted with a set of pre-amplifiers that includes a pre-amplifier specific for each target probe set and that can hybridize to the target probe pair that is hybridized to the respect target nucleic acid. Such an embodiment is illustrated schematically in FIG. 10A. The sample is also contacted with amplifiers, where the amplifiers include subsets of amplifiers specific for each pre-amplifier that is specific for a target probe pair that is specific for a target nucleic acid. Thus, each target nucleic acid has an assembly of unique components of an SGC, target probe pair(s), pre-amplifiers, and amplifiers, that provide discrimination between the target nucleic acids. In an additional embodiment, a pre-pre-amplifier can bind to a target probe pair as an additional amplification layer between the target probe pairs and the pre-amplifier (see FIGS. 9B and 10B).

In another embodiment, a sample is contacted with target probe sets comprising a pair of target probes that can specifically hybridize to a target nucleic acid. The sample is also contacted with a set of pre-pre-amplifiers that includes a pair of pre-pre-amplifiers specific for each target probe set and that can hybridize to the target probe pair that is hybridized to the respect target nucleic acid. Such an embodiment is illustrated schematically in FIG. 10C. The sample is also contacted with a set of pre-amplifiers that includes a pre-amplifier that can specifically bind to both pairs of pre-pre-amplifiers that are specific for a pair of target probes that are specific for a target. The sample is also contacted with amplifiers, where the amplifiers include subsets of amplifiers specific for each pre-amplifier that is specific for a pair of pre-pre-amplifiers that are specific for a target probe pair that is specific for a target nucleic acid. Thus, each target nucleic acid has an assembly of unique components of an SGC, target probe pairs, pre-pre-amplifiers, pre-amplifiers, and amplifiers, that provide discrimination between the target nucleic acids.

In order to detect the target nucleic acids, sets of label probes are contacted with the sample. Instead of contacting the sample with label probes that can detect all of the target nucleic acids, the sample is contacted with a set of label probes that can detect a subset of the target nucleic acids. Thus, rather than detecting all of the target nucleic acids at once, the target nucleic acids are detected in iterative rounds of detection. Within one round, the label probes specific for the respective target nucleic acids are distinguishable from each other, so that all of the target nucleic acids associated with a first round of applied label probes can be detected concurrently.

The number of target nucleic acids that can be detected concurrently in a single round will depend on the type of label used in the label probes and how such labels can be distinguished. For example, in the case of using fluorescent labels, the fluorophores used in a single round need to be distinguishable, so there should be spectral separation of the emissions of the fluorophores. The number of fluorophores that can be distinguished concurrently is up to 10, depending on the detection system and the availability of filters and/or software that can be used to distinguish fluorophores with overlapping emissions, which are considered to have spectral separation if they can be distinguished, as is well known in the art. Imaging systems for detecting multiple fluorescent labels are well known in the art (for example, Vectra Polaris, Perkin Elmer, Waltham Mass.).

In still another embodiment, the methods of the invention can be applied to simultaneous detection of double stranded nucleic acids and single stranded nucleic acids, for example, detection of DNA and RNA in the same sample. In such a case, probes can be designed to detect single stranded nucleic acids, such as RNA (see, for example, U.S. Pat. No. 7,709,198, U.S. publications 2008/0038725 and 2009/0081688, and 2017/0101672) and double stranded nucleic acids such that both double stranded nucleic acids and single stranded nucleic acids, such as DNA and RNA, can be detected in the same sample.

In some embodiments, each target probe set that is specific for a target nucleic acid comprises two or more pairs of target probes that specifically hybridize to the same target nucleic acid. In such a case, the pairs of target probes in the target probe set specific for a target nucleic acid bind to different and non-overlapping sequences of the target nucleic acid. When a target probe set is used that has two or more pairs of target probes that can specifically hybridize to the same target nucleic acid, the molecule that binds to the target probe pairs, either a pre-amplifier (see FIGS. 9A and 10A), or a pre-pre-amplifier (see FIGS. 9B, 9C, 10B and 10C), generally are the same for target probe pairs in the same target probe set. Thus, the target probe pairs that bind to the same target nucleic acid can be designed to comprise the same binding site for the molecule in the SGC that binds to the target probe pairs, that is, a pre-amplifier or pre-pre-amplifier. The use of multiple target probe pairs to detect a target nucleic acid provides for a higher signal associated with the assembly of multiple SGCs on the same target nucleic acid. In some embodiments, the number of target probe pairs used for binding to the same target nucleic acid are in the range of 1-10, 1-20, 1-30, 1-40, 1-50, 1-60, 1-70, 1-80, 1-90, 1-100, 1-110, 1-120, 1-130, 1-140, 1-150, 1-160, 1-170, 1-180, 1-190, or 1-200 pairs per target, or larger numbers of pairs, or any integer number of pairs in between, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, and the like.

The methods of the invention can be utilized to achieve the detection of desired target nucleic acids. In one embodiment, a target nucleic acid is detected with a plurality of target probe pairs. In such a case, target probe pairs are designed to bind to more than one region of a target nucleic acid to allow for the assembly of multiple SGCs onto a target nucleic acid. It is understood that the target binding sites of one target probe pair do not overlap with the target binding sites of another target probe pair if a plurality of target probe pairs are being used to bind to the same target nucleic acid.

In an embodiment of the invention, the target nucleic acids detected by the methods of the invention can be any nucleic acid present in the cell sample, including but not limited to, RNA, including messenger RNA (mRNA), micro RNA (miRNA), ribosomal RNA (rRNA), mitochondrial RNA, non-coding RNA, and the like, or DNA, and the like. In a particular embodiment, the nucleic acid is RNA. In the methods of the invention for multiplex detection of nucleic acids, it is understood the target nucleic acids can independently be DNA or RNA. In other words, the target nucleic acids to be detected can be, but are not necessarily, the same type of nucleic acid. Thus, the target nucleic acids to be detected in an assay of the invention can be DNA and RNA. In the case where the target nucleic acids are RNA, it is understood that the target nucleic acids can independently be selected from the group consisting of messenger RNA (mRNA), micro RNA (miRNA), ribosomal RNA (rRNA), mitochondrial RNA, and non-coding RNA. Thus, the target nucleic acids can independently be DNA or any type of RNA.

As described herein, the methods of the invention generally relate to in situ detection of target nucleic acids. Methods for in situ detection of nucleic acids are well known to those skilled in the art (see, for example, US 2008/0038725; US 2009/0081688; Hicks et al., J. Mol. Histol. 35:595-601 (2004)). As used herein, “in situ hybridization” or “ISH” refers to a type of hybridization that uses a directly or indirectly labeled complementary DNA or RNA strand, such as a probe, to bind to and localize a specific nucleic acid, such as DNA or RNA, in a sample, in particular a portion or section of tissue or cells (in situ). The probe types can be double stranded DNA (dsDNA), single stranded DNA (ssDNA), single stranded complimentary RNA (sscRNA), messenger RNA (mRNA), micro RNA (miRNA), ribosomal RNA, mitochondrial RNA, and/or synthetic oligonucleotides. The term “fluorescent in situ hybridization” or “FISH” refers to a type of ISH utilizing a fluorescent label. The term “chromogenic in situ hybridization” or “CISH” refers to a type of ISH with a chromogenic label. ISH, FISH and CISH methods are well known to those skilled in the art (see, for example, Stoler, Clinics in Laboratory Medicine 10(1):215-236 (1990); In situ hybridization. A practical approach, Wilkinson, ed., IRL Press, Oxford (1992); Schwarzacher and Heslop-Harrison, Practical in situ hybridization, BIOS Scientific Publishers Ltd, Oxford (2000)).

For methods of the invention for in situ detection of nucleic acid targets in a cell, including but not limited to in situ hybridization or flow cytometry, the cell is optionally fixed and/or permeabilized before hybridization of the target probes. Fixing and permeabilizing cells can facilitate retaining the nucleic acid targets in the cell and permit the target probes, label probes, amplifiers, pre-amplifiers, pre-pre-amplifiers, and so forth, to enter the cell and reach the target nucleic acid molecule. The cell is optionally washed to remove materials not captured to a nucleic acid target. The cell can be washed after any of various steps, for example, after hybridization of the target probes to the nucleic acid targets to remove unbound target probes, after hybridization of the pre-pre-amplifiers, pre-amplifiers, amplifiers, and/or label probes to the target probes, and the like. Methods for fixing and permeabilizing cells for in situ detection of nucleic acids, as well as methods for hybridizing, washing and detecting target nucleic acids, are also well known in the art (see, for example, US 2008/0038725; US 2009/0081688; Hicks et al., J. Mol. Histol. 35:595-601 (2004); Stoler, Clinics in Laboratory Medicine 10(1):215-236 (1990); In situ hybridization. A practical approach, Wilkinson, ed., IRL Press, Oxford (1992); Schwarzacher and Heslop-Harrison, Practical in situ hybridization, BIOS Scientific Publishers Ltd, Oxford (2000); Shapiro, Practical Flow Cytometry 3rd ed., Wiley-Liss, New York (1995); Ormerod, Flow Cytometry, 2nd ed., Springer (1999)). Exemplary fixing agents include, but are not limited to, aldehydes (formaldehyde, gluteraldehyde, and the like), acetone, alcohols (methanol, ethanol, and the like). Exemplary permeabilizing agents include, but are not limited to, alcohols (methanol, ethanol, and the like), acids (glacial acetic acid, and the like), detergents (Triton, NP-40, Tween™ 20, and the like), saponin, digitonin, Leucoperm™ (BioRad, Hercules, Calif.), and enzymes (for example, lysozyme, lipases, proteases and peptidases). Permeabilization can also occur by mechanical disruption, such as in tissue slices.

For in situ detection of double stranded nucleic acids, generally the sample is treated to denature the double stranded nucleic acids in the sample to provide accessibility for the target probes to bind by hybridization to a strand of the target double stranded nucleic acid. Conditions for denaturing double stranded nucleic acids are well known in the art, and include heat and chemical denaturation, for example, with base (NaOH), formamide, dimethyl sulfoxide, and the like (see Wang et al., Environ. Health Toxicol. 29:e2014007 (doi: 10.5620/eht.2014.29.e2014007) 2014; Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, Md. (1999)). For example, NaOH, LiOH or KOH, or other high pH buffers (pH>11) can be used to denature double stranded nucleic acids such as DNA. In addition, heat and chemical denaturation methods can be used in combination.

Such in situ detection methods can be used on tissue specimens immobilized on a glass slide, on single cells in suspension such as peripheral blood mononucleated cells (PBMCs) isolated from blood samples, and the like. Tissue specimens include, for example, tissue biopsy samples. Blood samples include, for example, blood samples taken for diagnostic purposes. In the case of a blood sample, the blood can be directly analyzed, such as in a blood smear, or the blood can be processed, for example, lysis of red blood cells, isolation of PBMCs or leukocytes, isolation of target cells, and the like, such that the cells in the sample analyzed by methods of the invention are in a blood sample or are derived from a blood sample. Similarly, a tissue specimen can be processed, for example, the tissue specimen minced and treated physically or enzymatically to disrupt the tissue into individual cells or cell clusters. Additionally, a cytological sample can be processed to isolate cells or disrupt cell clusters, if desired. Thus, the tissue, blood and cytological samples can be obtained and processed using methods well known in the art. The methods of the invention can be used in diagnostic applications to identify the presence or absence of pathological cells based on the presence or absence of a nucleic acid target that is a biomarker indicative of a pathology.

It is understood by those skilled in the art that any of a number of suitable samples can be used for detecting target nucleic acids using methods of the invention. The sample for use in methods of the invention will generally be a biological sample or tissue sample. Such a sample can be obtained from a biological subject, including a sample of biological tissue or fluid origin that is collected from an individual or some other source of biological material such as biopsy, autopsy or forensic materials. A biological sample also includes samples from a region of a biological subject containing or suspected of containing precancerous or cancer cells or tissues, for example, a tissue biopsy, including fine needle aspirates, blood sample or cytological specimen. Such samples can be, but are not limited to, organs, tissues, tissue fractions and/or cells isolated from an organism such as a mammal. Exemplary biological samples include, but are not limited to, a cell culture, including a primary cell culture, a cell line, a tissue, an organ, an organelle, a biological fluid, and the like. Additional biological samples include but are not limited to a skin sample, tissue biopsies, including fine needle aspirates, cytological samples, stool, bodily fluids, including blood and/or serum samples, saliva, semen, and the like. Such samples can be used for medical or veterinary diagnostic purposes. A sample can also be obtained from other sources, for example, food, soil, surfaces of objects, and the like, and other materials for which detection of target nucleic acids is desired. Thus, the methods of the invention can be used for detection of one or more pathogens, such as a virus, a bacterium, a fungus, a single celled organism such as a parasite, and the like, from a biological sample obtained from an individual or other sources.

Collection of cytological samples for analysis by methods of the invention are well known in the art (see, for example, Dey, “Cytology Sample Procurement, Fixation and Processing” in Basic and Advanced Laboratory Techniques in Histopathology and Cytology pp. 121-132, Springer, Singapore (2018); “Non-Gynecological Cytology Practice Guideline” American Society of Cytopathology, Adopted by the ASC executive board Mar. 2, 2004). Methods for processing samples for analysis of cervical tissue, including tissue biopsy and cytology samples, are well known in the art (see, for example, Cecil Textbook of Medicine, Bennett and Plum, eds., 20th ed., WB Saunders, Philadelphia (1996); Colposcopy and Treatment of Cervical Intraepithelial Neoplasia: A Beginner's Manual, Sellors and Sankaranarayanan, eds., International Agency for Research on Cancer, Lyon, France (2003); Kalaf and Cooper, J. Clin. Pathol. 60:449-455 (2007); Brown and Trimble, Best Pract. Res. Clin. Obstet. Gynaecol. 26:233-242 (2012); Waxman et al., Obstet. Gynecol. 120:1465-1471 (2012); Cervical Cytology Practice Guidelines TOC, Approved by the American Society of Cytopathology (ASC) Executive Board, Nov. 10, 2000)). In one embodiment, the cytological sample is a cervical sample, for example, a pap smear. In one embodiment, the sample is a fine needle aspirate.

In particular embodiments of the invention, the sample is a tissue specimen or is derived from a tissue specimen. In other particular embodiments of the invention, the sample is a blood sample or is derived from a blood sample. In still other particular embodiments of the invention, the sample is a cytological sample or is derived from a cytological sample.

The invention is based on building a complex between a target nucleic acid in order to label the target nucleic acid with a detectable label. Such a complex is sometimes referred to as a signal generating complex (SGC; see, for example, US 20170101672). Such a complex, or SGC, is achieved by building layers of molecules that allow the attachment of a large number of labels to a target nucleic acid.

The methods of the invention can employ a signal generating complex (SGC), where the SGC comprises multiple molecules rather than a single molecule. Such an SGC is particularly useful for amplifying the detectable signal, providing higher sensitivity detection of target nucleic acids. Such methods for amplifying a signal are described, for example, in U.S. Pat. Nos. 5,635,352, 5,124,246, 5,710,264, 5,849,481, and 7,709,198, and U.S. publications 2008/0038725 and 2009/0081688, as well as WO 2007/001986 and WO 2012/054795, each of which is incorporated herein by reference. The generation of an SGC is a principle of the RNAscope™ assay (see U.S. Pat. Nos. 7,709,198, 8,658,361 and 9,315,854, U.S. publications 2008/0038725, 2009/0081688 and 2016/0201117, as well as WO 2007/001986 and WO 2012/054795, each of which is incorporated herein by reference).

A basic Signal Generating Complex (SGC) is illustrated in FIG. 9A (see also US 2009/0081688, which is incorporated herein by reference). A pair of target probes, depicted in FIG. 9 as a pair of “Z's”, hybridizes to a complementary molecule sequence, labeled “Target”. Each target probe contains an additional sequence complementary to a pre-amplifier molecule (PA, illustrated in green), which must hybridize simultaneously to both members of the target probe pair in order to bind stably. The pre-amplifier molecule is made up of two domains: one domain with a region that hybridizes to each target probe, and one domain that contains a series of nucleotide sequence repeats, each complementary to a sequence on the amplifier molecule (Amp, illustrated in black). The presence of multiple repeats of this sequence allows multiple amplifier molecules to hybridize to one pre-amplifier, which increases the overall signal amplification. Each amplifier molecule is made up of two domains, one domain with a region that hybridizes to the pre-amplifier, and one domain that contains a series of nucleotide sequence repeats, each complementary to a sequence on the label probe (LP, illustrated in yellow), allowing multiple label probes to hybridize to each amplifier molecule, further increasing the total signal amplification. Each label probe contains two components. One component is made up of a nucleotide sequence complementary to the repeat sequence on the amplifier molecule to allow the label probe to hybridize. This nucleotide sequence is linked to the second component, which can be any signal-generating entity, including a fluorescent or chromogenic label for direct visualization, a directly detectable metal isotope, or an enzyme or other chemical capable of facilitating a chemical reaction to generate a fluorescent, chromogenic, or other detectable signal, as described herein. In FIG. 9A, the label probe is depicted as a line, representing the nucleic acid component, and a star, representing the signal-generating component. Together, the assembly from target probe to label probe is referred to as a Signal Generating Complex (SGC).

FIG. 9B illustrates a SGC enlarged by adding an amplification molecule layer, in this case a pre-pre-amplifier molecule (PPA, shown in red). The PPA binds to both target probes in one domain and multiple pre-amplifiers (PAs) in another domain.

FIG. 9C illustrates a different SGC structure that uses collaborative hybridization at the pre-amplifier level (see US 2017/0101672, which is incorporated herein by reference). Similarly to the SGC formed in FIGS. 9A and 9B, a pair of target probes hybridize to the target molecule sequence. Each target probe contains an additional sequence complementary to a unique pre-pre-amplifier molecule (PPA-1, illustrated in purple; PPA-2, illustrated in red). The use of two independent molecules sets up a base on which collaborative hybridization can be required. Each pre-pre-amplifier molecule is made up of two domains, one domain with a region that hybridizes to one of the target probes, and one domain that contains a series of nucleotide sequence repeats, each containing both a sequence complementary to a sequence within the pre-amplifier molecule (PA, illustrated in green), as well as a spacer sequence to facilitate PPA-PA binding efficiency. To stably attach to the growing SGC, each PA must hybridize to both PPA molecules simultaneously. Each pre-amplifier molecule is made up of two domains, one domain that contains sequences complementary to both pre-pre-amplifiers to allow hybridization, and one domain that contains a series of nucleotide sequence repeats each complementary to a sequence on the amplifier molecule (AMP, illustrated in black). Multiple repeats of the amplifier hybridization sequence allows multiple amplifier molecules to hybridize to each pre-amplifier, further increasing signal amplification. For simplicity of illustration, amplifier molecules are shown hybridizing to one pre-amplifier molecule, but it is understood that amplifiers can bind to each pre-amplifier. Each amplifier molecule contains a series of nucleotide sequence repeats complementary to a sequence within the label probe (LP, illustrated in yellow), allowing several label probes to hybridize to each amplifier molecule. Each label probe contains a signal-generating element to provide for signal detection.

As described above, whether using a configuration as depicted in FIG. 9A, 9B, 10A or 10B, or a configuration as depicted in FIGS. 9C and 10C, the components of the SGC are designed such that the binding of both target probes is required in order to build an SGC. In the case of the configuration of FIG. 9A, 9B, 10A or 10B, a pre-amplifier (or pre-pre-amplifier in FIGS. 9B and 10B) must bind to both members of the target probe pair for stable binding to occur. This is achieved by designing binding sites between the target probes and the pre-amplifier (or pre-pre-amplifier) such that binding of both target probes to the pre-amplifier (or pre-pre-amplifier) has a higher melting temperature (Tm) than the binding of a single target probe to the pre-amplifier (or pre-pre-amplifier), and where the binding of a single target probe is unstable under the conditions of the assay. This design has been described previously, for example, in U.S. Pat. No. 7,709,198, U.S. publications 2008/0038725 and 2009/0081688, WO 2007/001986 WO 2007/002006, Wang et al., supra, 2012, Anderson et al., supra, 2016). By configuring the SGC components this way, the assembly of the SGC is achieved when both target probes are bound to the target nucleic acid and the pre-amplifier, thereby reducing background noise since assembly of an SGC as a false positive is minimized.

In the case of the configuration of FIGS. 9C and 10C, the requirement that an SGC be formed only when both members of a target probe pair are bound to the target nucleic acid is achieved by requiring that a pre-amplifier be bound to both pre-pre-amplifiers, which in turn are bound to both members of the target probe pair, respectively. This requirement is achieved by designing the binding sites between the pre-pre-amplifiers and the pre-amplifier such that the melting temperature (Tm) between the binding of both pre-pre-amplifiers to the pre-amplifier is higher than the melting temperature of either pre-pre-amplifier alone, and where the binding of one of the pre-pre-amplifiers to the pre-amplifier is unstable under the conditions of the assay. This design has been described previously, for example, in US 20170101672, WO 2017/066211 and Baker et al., supra, 2017). Unless the pre-amplifier is bound to both pre-pre-amplifiers, the amplifiers and label probes cannot assemble into an SGC bound to the target nucleic acid, thereby reducing background noise since assembly of an SGC as a false positive is minimized.

As disclosed herein, the methods can be based on building a signal-generating complex (SGC) bound to a target nucleic acid in order to detect the presence of the target nucleic acid in the cell. The components for building an SGC generally comprise nucleic acids such that nucleic acid hybridization reactions are used to bind the components of the SGC to the target nucleic acid. Methods of selecting appropriate regions and designing specific and selective reagents that bind to the target nucleic acids, in particular oligonucleotides or probes that specifically and selectively bind to a target nucleic acid, or other components of the SGC, are well known to those skilled in the art (see Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, Md. (1999)). The target probes are designed such that the probes specifically hybridize to a target nucleic acid. A desired specificity can be achieved using appropriate selection of regions of a target nucleic acid as well as appropriate lengths of a binding agent such as an oligonucleotide or probe, and such selection methods are well known to those skilled in the art. Thus, one skilled in the art will readily understand and can readily determine appropriate reagents, such as oligonucleotides or probes, that can be used to target one particular target nucleic acid over another target nucleic acid, or to provide binding to the components of the SGC. Similar specificity can be achieved for a target-specific SGC by using appropriate selection of unique sequences such that a given component of a target-specific SGC (for example, target probe, pre-pre-amplifier, pre-amplifier, amplifier, label probe) will bind to the respective components such that the SGC is bound to a specific target (see FIG. 10).

As described herein, embodiments of the invention include the use of target probe pairs. In the case where a pair of target probes binds to the same pre-amplifier (FIGS. 9A and 10A) or pre-pre-amplifier (FIGS. 9B and 10B), a probe configuration, sometimes referred to as a “Z” configuration, can be used. Such a configuration and its advantages for increasing sensitivity and decreasing background are described, for example, in U.S. Pat. No. 7,709,198, U.S. publications 2008/0038725 and 2009/0081688, and WO 2007/001986 and WO 2007/002006, each of which is incorporated herein by reference. U.S. Pat. No. 7,709,198 and U.S. publications 2008/0038725 and 2009/0081688 additionally describe details for selecting characteristics of the target probes, such as target probe pairs, including length, orientation, hybridization conditions, and the like. One skilled in the art can readily identify suitable configurations based on the teachings herein and, for example, in U.S. Pat. No. 7,709,198, U.S. publications 2008/0038725 and 2009/0081688, and WO 2007/001986 and WO 2007/002006.

As described herein, the target binding site of the target probes in a target probe pair can be in any desired orientation and combination. For example, the target binding site of one member of the target probe pair can be 5′ or 3′ to the pre-amplifier or pre-pre-amplifier binding site, and the other member of the pair can independently be oriented with the target binding site 5′ or 3′ to the pre-amplifier or pre-pre-amplifier binding site.

In another embodiment, the SGC used to detect the presence of a target nucleic acid is based on a collaborative hybridization of one or more components of the SGC (see US 20170101672 and WO 2017/066211, each of which is incorporated herein by reference). Such a collaborative hybridization is also referred to herein as BaseScope™. In a collaborative hybridization effect, the binding between two components of an SGC is mediated by two binding sites, and the melting temperature of the binding to the two sites simultaneously is higher than the melting temperature of the binding of one site alone (see US 20170101672 and WO 2017/066211). The collaborative hybridization effect can be enhanced by target probe set configurations as described in US 20170101672 and WO 2017/066211.

The methods of the invention, and related compositions, can utilize collaborative hybridization to increase specificity and to reduce background in in situ detection of nucleic acid targets, where a complex physiochemical environment and the presence of an overwhelming number of non-target molecules can generate high noise. Using such a collaborative hybridization method, the binding of label probes only occurs when the SGC is bound to the target nucleic acid. As described in US 20170101672 and WO 2017/066211 and illustrated in FIG. 1 thereof, the method can be readily modified to provide a desired signal to noise ratio by increasing the number of collaborative hybridizations in one or more components of the SGC.

In another embodiment, the collaborative hybridization can be applied to various components of the SGC. For example, the binding between components of an SGC can be a stable reaction, as described herein, or the binding can be configured to require a collaborative hybridization, also as described herein. In such a case, the binding component intended for collaborative hybridization are designed such that the component contains two segments that bind to another component.

Thus, the methods for detecting a target nucleic acid can utilize collaborative hybridization for the binding reactions between any one or all of the components in the detection system that provides an SGC specifically bound to a target nucleic acid. The number of components, and which components, to apply collaborative hybridization can be selected based on the desired assay conditions, the type of sample being assayed, a desired assay sensitivity, and so forth. Any one or combination of collaborative hybridization binding reactions can be used to increase the sensitivity and specificity of the assay. In embodiments of the invention, the collaborative hybridization can be between a pre-pre-amplifier and a pre-amplifier, between a pre-amplifier and an amplifier, between an amplifier and a label probe, or combinations thereof (see, for example, US 20170101672 and WO 2017/066211).

As disclosed herein, the components are generally bound directly to each other. In the case of nucleic acid containing components, the binding reaction is generally by hybridization. In the case of a hybridization reaction, the binding between the components is direct. If desired, an intermediary component can be included such that the binding of one component to another is indirect, for example, the intermediary component contains complementary binding sites to bridge two other components.

As described herein, the configuration of various components can be selected to provide a desired stable or collaborative hybridization binding reaction (see, for example, US 20170101672). It is understood that, even if a binding reaction is exemplified herein as a stable or unstable reaction, such as for a collaborative hybridization, any of the binding reactions can be modified, as desired, so long as the target nucleic acid is detected. It is further understood that the configuration can be varied and selected depending on the assay and hybridization conditions to be used. In general, if a binding reaction is desired to be stable, the segments of complementary nucleic acid sequence between the components is generally in the range of 10 to 50 nucleotides, or greater, for example, 16 to 30 nucleotides, such as 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides, or greater. If a binding reaction is desired to be relatively unstable, such as when a collaborative hybridization binding reaction is employed, the segments of complementary nucleic acid sequence between the components is generally in the range of 5 to 18 nucleotides, for example, 5, 6, 7, 8, 9, 10, 11, 12, 13, 14, 15, 16, 17 or 18 nucleotides. It is understood that the nucleotide lengths can be somewhat shorter or longer for a stable or unstable hybridization, depending on the sequence (for example, GC content) and the conditions employed in the assay. It is further understood, as disclosed herein, that modified nucleotides such as Locked Nucleic Acid (LNA) or Bridged Nucleic Acid (BNA) can be used to increase the binding strength at the modified base, thereby allowing length of the binding segment to be reduced. Thus, it is understood that, with respect to the length of nucleic acid segments that are complementary to other nucleic acid segments, the lengths described herein can be reduced further, if desired. A person skilled in the art can readily determine appropriate probe designs, including length, the presence of modified nucleotides, and the like, to achieve a desired interaction between nucleic acid components.

In designing binding sites between two nucleic acid sequences comprising complementary sequences, the complementary sequences can optionally be designed to maximize the difference in melting temperature (dTₘ). This can be done by using melting temperature calculation algorithms known in the art (see, for example, SantaLucia, Proc. Natl. Acad. Sci. U.S.A. 95:1460-1465 (1998)). In addition, artificial modified bases such as Locked Nucleic Acid (LNA) or bridged nucleic acid (BNA) and naturally occurring 2′-O-methyl RNA are known to enhance the binding strength between complementary pairs (Petersen and Wengel, Trends Biotechnol. 21:74-81 (2003); Majlessi et al., Nucl. Acids Res. 26:2224-2229 (1998)). These modified bases can be strategically introduced into the binding site between components of an SGC, as desired.

One approach is to utilize modified nucleotides (LNA, BNA or 2′-O-methyl RNA). Because each modified base can increase the melting temperature, the length of binding regions between two nucleic acid sequences (i.e., complementary sequences) can be substantially shortened. The binding strength of a modified base to its complement is stronger, and the difference in melting temperatures (dTₘ) is increased. Yet another embodiment is to use three modified bases (for example, three LNA, BNA or 2′-O-methyl RNA bases, or a combination of two or three different modified bases) in the complementary sequences of a nucleic acid component or between two nucleic acid components, for example of a signal generating complex (SGC), that are to be hybridized. Such components can be, for example, a pre-pre-amplifier, a pre-amplifier, an amplifier, a label probe, or a pair of target probes.

The modified bases, such as LNA or BNA, can be used in the segments of selected components of SGC, in particular those mediating binding between nucleic acid components, which increases the binding strength of the base to its complementary base, allowing a reduction in the length of the complementary segments (see, for example, Petersen and Wengel, Trends Biotechnol. 21:74-81 (2003); U.S. Pat. No. 7,399,845). Artificial bases that expand the natural 4-letter alphabet such as the Artificially Expanded Genetic Information System (AEGIS; Yang et al., Nucl. Acids Res. 34 (21): 6095-6101 (2006)) can be incorporated into the binding sites among the interacting components of the SGC. These artificial bases can increase the specificity of the interacting components, which in turn can allow lower stringency hybridization reactions to yield a higher signal.

With respect to a target probe pair, the target probe pair can be designed to bind to immediately adjacent segments of the target nucleic acid or on segments that have one to a number of bases between the target probe binding sites of the target probe pair. Generally, target probe pairs are designed for binding to the target nucleic acid such that there are generally between 0 to 500 bases between the binding sites on the target nucleic acid, for example, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 420, 440, 460, 480, or 500 bases, or any integer length in between. In particular embodiments, the binding sites for the pair of target probes are between 0 to 100, 0 to 200, or 0 to 300 bases, or any integer length in between. In the case where more than one target probe pair is used in a target probe set to bind to the same target nucleic acid that is RNA or single stranded DNA, and where there is a gap in the binding sites between a pair of target probes, it is understood that the binding sites of different target probe pairs do not overlap. In the case of detecting double stranded nucleic acids, such as DNA, some overlap between different target probe pairs can occur, so long as the target probe pairs are able to concurrently bind to the respective binding sites of the double stranded target nucleic acid.

The SGC also comprises a plurality of label probes (LPs). Each LP comprises a segment that is detectable. The detectable component can be directly attached to the LP, or the LP can hybridize to another nucleic acid that comprises the detectable component, i.e., the label. As used herein, a “label” is a moiety that facilitates detection of a molecule. Common labels in the context of the present invention include fluorescent, luminescent, light-scattering, and/or colorimetric labels. Suitable labels include enzymes, and fluorescent and chromogenic moieties, as well as radionuclides, substrates, cofactors, inhibitors, chemiluminescent moieties, magnetic particles, rare earth metals, metal isotopes, and the like. In a particular embodiment of the invention, the label is an enzyme. Exemplary enzyme labels include, but are not limited to Horse Radish Peroxidase (HRP), Alkaline Phosphatase (AP), β-galactosidase, glucose oxidase, and the like, as well as various proteases. Other labels include, but are not limited to, fluorophores, Dinitrophenyl (DNP), and the like. Labels are well known to those skilled in the art, as described, for example, in Hermanson, Bioconjugate Techniques, Academic Press, San Diego (1996), and U.S. Pat. Nos. 3,817,837; 3,850,752; 3,939,350; 3,996,345; 4,277,437; 4,275,149; and 4,366,241. Many labels are commercially available and can be used in methods and assays of the invention, including detectable enzyme/substrate combinations (Pierce, Rockford Ill.; Santa Cruz Biotechnology, Dallas Tex.; Life Technologies, Carlsbad Calif.). In a particular embodiment of the invention, the enzyme can utilize a chromogenic or fluorogenic substrate to produce a detectable signal, as described herein. Exemplary labels are described herein.

Any of a number of enzymes or non-enzyme labels can be utilized so long as the enzymatic activity or non-enzyme label, respectively, can be detected. The enzyme thereby produces a detectable signal, which can be utilized to detect a target nucleic acid. Particularly useful detectable signals are chromogenic or fluorogenic signals. Accordingly, particularly useful enzymes for use as a label include those for which a chromogenic or fluorogenic substrate is available. Such chromogenic or fluorogenic substrates can be converted by enzymatic reaction to a readily detectable chromogenic or fluorescent product, which can be readily detected and/or quantified using microscopy or spectroscopy. Such enzymes are well known to those skilled in the art, including but not limited to, horseradish peroxidase, alkaline phosphatase, β-galactosidase, glucose oxidase, and the like (see Hermanson, Bioconjugate Techniques, Academic Press, San Diego (1996)). Other enzymes that have well known chromogenic or fluorogenic substrates include various peptidases, where chromogenic or fluorogenic peptide substrates can be utilized to detect proteolytic cleavage reactions. The use of chromogenic and fluorogenic substrates is also well known in bacterial diagnostics, including but not limited to the use of α- and β-galactosidase, β-glucuronidase, 6-phospho-β-D-galactoside 6-phosphogalactohydrolase, β-glucosidase, α-glucosidase, amylase, neuraminidase, esterases, lipases, and the like (Manafi et al., Microbiol. Rev. 55:335-348 (1991)), and such enzymes with known chromogenic or fluorogenic substrates can readily be adapted for use in methods of the present invention.

Various chromogenic or fluorogenic substrates to produce detectable signals are well known to those skilled in the art and are commercially available. Exemplary substrates that can be utilized to produce a detectable signal include, but are not limited to, 3,3′-diaminobenzidine (DAB), 3,3′,5,5′-tetramethylbenzidine (TMB), Chloronaphthol (4-CN)(4-chloro-1-naphthol), 2,2′-azino-bis(3-ethylbenzothiazoline-6-sulphonic acid) (ABTS), o-phenylenediamine dihydrochloride (OPD), and 3-amino-9-ethylcarbazole (AEC) for horseradish peroxidase; 5-bromo-4-chloro-3-indolyl-1-phosphate (BCIP), nitroblue tetrazolium (NBT), Fast Red (Fast Red TR/AS-MX), and p-Nitrophenyl Phosphate (PNPP) for alkaline phosphatase; 1-Methyl-3-indolyl-β-D-galactopyranoside and 2-Methoxy-4-(2-nitrovinyl)phenyl β-D-galactopyranoside for β-galactosidase; 2-Methoxy-4-(2-nitrovinyl)phenyl β-D-glucopyranoside for β-glucosidase; and the like. Exemplary fluorogenic substrates include, but are not limited to, 4-(Trifluoromethyl)umbelliferyl phosphate for alkaline phosphatase; 4-Methylumbelliferyl phosphate bis (2-amino-2-methyl-1,3-propanediol), 4-Methylumbelliferyl phosphate bis (cyclohexylammonium) and 4-Methylumbelliferyl phosphate for phosphatases; QuantaBlu™ and QuantaRed™ for horseradish peroxidase; 4-Methylumbelliferyl β-D-galactopyranoside, Fluorescein di(β-D-galactopyranoside) and Naphthofluorescein di-(β-D-galactopyranoside) for β-galactosidase; 3-Acetylumbelliferyl β-D-glucopyranoside and 4-Methylumbelliferyl-β-D-glucopyranoside for β-glucosidase; and 4-Methylumbelliferyl-α-D-galactopyranoside for α-galactosidase. Exemplary enzymes and substrates for producing a detectable signal are also described, for example, in US publication 2012/0100540. Various detectable enzyme substrates, including chromogenic or fluorogenic substrates, are well known and commercially available (Pierce, Rockford Ill.; Santa Cruz Biotechnology, Dallas Tex.; Invitrogen, Carlsbad Calif.; 42 Life Science; Biocare). Generally, the substrates are converted to products that form precipitates that are deposited at the site of the target nucleic acid. Other exemplary substrates include, but are not limited to, HRP-Green (42 Life Science), Betazoid DAB, Cardassian DAB, Romulin AEC, Bajoran Purple, Vina Green, Deep Space Black™, Warp Red™, Vulcan Fast Red and Ferangi Blue from Biocare (Concord Calif.; biocare.net/products/detection/chromogens).

Exemplary rare earth metals and metal isotopes suitable as a detectable label include, but are not limited to, lanthanide (III) isotopes such as 141Pr, 142Nd, 143Nd, 144Nd, 145Nd, 146Nd, 147Sm, 148Nd, 149Sm, 150Nd, 151Eu, 152Sm, 153Eu, 154Sm, 155Gd, 156Gd, 158Gd, 159Tb, 160Gd, 161Dy, 162Dy, 163Dy, 164Dy, 165Ho, 166Er, 167Er, 168Er, 169Tm, 170Er, 171Yb, 172Yb, 173Yb, 174Yb, 175Lu, and 176Yb. Metal isotopes can be detected, for example, using time-of-flight mass spectrometry (TOF-MS) (for example, Fluidigm Helios and Hyperion systems, fluidigm.com/systems; South San Francisco, Calif.).

Biotin-avidin (or biotin-streptavidin) is a well known signal amplification system based on the fact that the two molecules have extraordinarily high affinity to each other and that one avidin/streptavidin molecule can bind four biotin molecules. Antibodies are widely used for signal amplification in immunohistochemistry and ISH. Tyramide signal amplification (TSA) is based on the deposition of a large number of haptenized tyramide molecules by peroxidase activity. Tyramine is a phenolic compound. In the presence of small amounts of hydrogen peroxide, immobilized Horse Radish Peroxidase (HRP) converts the labeled substrate into a short-lived, extremely reactive intermediate. The activated substrate molecules then very rapidly react with and covalently bind to electron-rich moieties of proteins, such as tyrosine, at or near the site of the peroxidase binding site. In this way, many hapten molecules conjugated to tyramide can be introduced at the hybridization site in situ. Subsequently, the deposited tyramide-hapten molecules can be visualized directly or indirectly. Such a detection system is described in more detail, for example, in U.S. publication 2012/0100540.

Embodiments described herein can utilize enzymes to generate a detectable signal using appropriate chromogenic or fluorogenic substrates. It is understood that, alternatively, a label probe can have a detectable label directly coupled to the nucleic acid portion of the label probe. Exemplary detectable labels are well known to those skilled in the art, including but not limited to chromogenic or fluorescent labels (see Hermanson, Bioconjugate Techniques, Academic Press, San Diego (1996)). Exemplary fluorophores useful as labels include, but are not limited to, rhodamine derivatives, for example, tetramethylrhodamine, rhodamine B, rhodamine 6G, sulforhodamine B, Texas Red (sulforhodamine 101), rhodamine 110, and derivatives thereof such as tetramethylrhodamine-5-(or 6), lissamine rhodamine B, and the like; 7-nitrobenz-2-oxa-1,3-diazole (NBD); fluorescein and derivatives thereof; napthalenes such as dansyl (5-dimethylaminonapthalene-1-sulfonyl); coumarin derivatives such as 7-amino-4-methylcoumarin-3-acetic acid (AMCA), 7-diethylamino-3-[(4′-(iodoacetyl)amino)phenyl]-4-methylcoumarin (DCIA), Alexa fluor dyes (Molecular Probes), and the like; 4,4-difluoro-4-bora-3a,4a-diaza-s-indacene (BODIPY™) and derivatives thereof (Molecular Probes; Eugene, Oreg.); pyrenes and sulfonated pyrenes such as Cascade Blue™ and derivatives thereof, including 8-methoxypyrene-1,3,6-trisulfonic acid, and the like; pyridyloxazole derivatives and dapoxyl derivatives (Molecular Probes); Lucifer Yellow (3,6-disulfonate-4-amino-naphthalimide) and derivatives thereof; CyDye™ fluorescent dyes (Amersham/GE Healthcare Life Sciences; Piscataway N.J.); ATTO 390, DyLight 395XL, ATTO 425, ATTO 465, ATTO 488, ATTO 490LS, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740, Cyan 500 NETS-Ester (ATTO-TECH, Siegen, Germany), and the like. Exemplary chromophores include, but are not limited to, phenolphthalein, malachite green, nitroaromatics such as nitrophenyl, diazo dyes, dabsyl (4-dimethylaminoazobenzene-4′-sulfonyl), and the like.

As disclosed herein, the methods can utilize concurrent detection of multiple target nucleic acids. In the case of using fluorophores as labels, the fluorophores to be used for detection of multiple target nucleic acids are selected so that each of the fluorophores are distinguishable and can be detected concurrently in the fluorescence microscope in the case of concurrent detection of target nucleic acids. Such fluorophores are selected to have spectral separation of the emissions so that distinct labeling of the target nucleic acids can be detected concurrently. Methods of selecting suitable distinguishable fluorophores for use in methods of the invention are well known in the art (see, for example, Johnson and Spence, “Molecular Probes Handbook, a Guide to Fluorescent Probes and Labeling Technologies, 11th ed., Life Technologies (2010)).

Well known methods such as microscopy, cytometry (e.g., mass cytometry, cytometry by time of flight (CyTOF), flow cytometry) or spectroscopy can be utilized to visualize chromogenic, fluorescent, or metal detectable signals associated with the respective target nucleic acids. In general, either chromogenic substrates or fluorogenic substrates, or chromogenic or fluorescent labels, or rare earth or metal isotopes, will be utilized for a particular assay, if different labels are used in the same assay, so that a single type of instrument can be used for detection of nucleic acid targets in the same sample.

The invention described herein generally relates to detection of multiple target nucleic acids in a sample. It is understood that the methods of the invention can additionally be applied to detecting multiple target nucleic acids and optionally other molecules in the sample, in particular in the same cell as the target nucleic acid. For example, in addition to detecting multiple target nucleic acids, proteins expressed in a cell can also concurrently be detected using a similar rationale as described herein for detecting target nucleic acids. In this case, in one or more rounds of detection of multiple target nucleic acids, and optionally one or more proteins expressed in a cell can be detected, for example, by using a detectable label to detect the protein. If the protein is being detected in an earlier round of target nucleic acid detection, the protein can be detected with a cleavable label, similar to that used for detecting target nucleic acids. If the protein is being detected in the last round of detection, the label does not need to be cleavable. Detection of proteins in a cell are well known to those skilled in the art, for example, by detecting the binding of protein-specific antibodies using any of the well known detection systems, including those described herein for detection of target nucleic acids. Detection of target nucleic acids and protein in the same cell has been described (see also Schulz et al., Cell Syst. 6(1):25-36 (2018)).

It is understood that the invention can be carried out in any desired order, so long as the target nucleic acids are detected. Thus, in a method of the invention, the steps of contacting a cell with any components for assembly of an SGC can be performed in any desired order, can be carried out sequentially, or can be carried out simultaneously, or some steps can be performed sequentially while others are performed simultaneously, as desired, so long as the target nucleic acids are detected. It is further understood that embodiments disclosed herein can be independently combined with other embodiments disclosed herein, as desired, in order to utilize various configurations, component sizes, assay conditions, assay sensitivity, and the like.

It is understood that the invention can be carried out in any format that provides for the detection of a target nucleic acid. Although implementation of the invention has generally been described herein using in situ hybridization, it is understood that the invention can be carried out for detection of target nucleic acids in other formats, in particular for detection of target nucleic acids in a cell, as are well known in the art. One method that can be used for detecting target nucleic acids in a cell is flow cytometry, as is well known in the art (see, for example, Shapiro, Practical Flow Cytometry 3rd ed., Wiley-Liss, New York (1995); Ormerod, Flow Cytometry, 2nd ed., Springer (1999)). The methods, samples and kits of the invention can thus be used in an in situ hybridization assay format or another format, such as flow cytometry. The application of nucleic acid detection methods, including in situ hybridization, to flow cytometry has been described previously (see, for example, Hanley et al., PLoS One, 8(2):e57002. doi: 10.1371/journal.pone.0057002 (2013); Baxter et al., Nature Protocols 12(10):2029-2049 (2017)).

In some cases, it can be desirable to reduce the number of assay steps, for example, reduce the number of hybridization and wash steps. One way of reducing the number of assay steps is to pre-assemble some or all components of the SGC prior to contacting with a cell. Such a pre-assembly can be performed by hybridizing some or all of the components of the SGC together prior to contacting the target nucleic acid.

The invention also provides a sample comprising a cell or a plurality of cells. The cell can optionally be fixed. The cells can optionally be permeabilized. Fixing and/or permeabilizing cells is particularly applicable to in situ hybridization assays.

In one embodiment, the invention provides a sample comprising a cell, comprising (A) at least one cell containing a plurality of target nucleic acids; and (B) set of probes, wherein the set of probes comprises subsets of probes comprising a plurality detectable labels that provide unique labeling of each target nucleic acid, wherein each probe subset comprises one or more distinct labels, wherein the number and/or combination of distinct labels is unique for each target nucleic acid, and wherein at least one subset of probes is specifically hybridized to a target nucleic acid.

In one embodiment of a sample of the invention, each probe in each of the probe subsets comprises (a) a set of target probes, wherein the target probe set comprises one or more subsets of target probes, wherein each target probe subset comprises a plurality of pairs of target probes that specifically hybridize to a target nucleic acid, wherein the target probes of a subset are hybridized to the target nucleic acid; (b) a set of pre-amplifiers, wherein the pre-amplifier set comprises one or more subsets of pre-amplifiers, wherein the one or more pre-amplifier subsets comprise a pre-amplifier specific for each of the target probe pairs in the one or more target probe subsets, wherein each pre-amplifier comprises binding sites for the pair of target probes of one of the target probe subsets and a plurality of binding sites for an amplifier, wherein the pre-amplifiers of a subset are hybridized to the respective target probe subset; (c) a set of amplifiers, wherein the amplifier set comprises one or more subsets of amplifiers specific for each pre-amplifier subset, wherein each amplifier subset comprises a plurality of amplifiers, wherein the amplifiers of one of the amplifier subsets comprise a binding site for the pre-amplifiers of one of the pre-amplifier subsets and a plurality of binding sites for a label probe, wherein the amplifiers of a subset are hybridized to the respective pre-amplifier subset; and (d) a set of label probes, wherein the label probe set comprises one or more subsets of label probes, wherein each label probe subset is specific for one of the amplifier subsets, wherein each label probe subset comprises a plurality of label probes, wherein the label probes in each of the label probe subsets comprise a label and a binding site for the amplifiers of one of the amplifier subsets, wherein the labels in each label probe subset are distinguishable between the label probe subsets, wherein the label probes of a subset are hybridized to the respective amplifier subset; wherein the one or more label probe subsets in each probe subset specific for a target nucleic acid comprise at least one label or a combination of labels that is different for each probe subset.

In one embodiment of the sample, the set of target probes, pre-amplifiers, amplifiers and label probes each comprise two or more subsets. In another embodiment, the set of target probes, pre-amplifiers, amplifiers and label probes each comprise three or more subsets. In another embodiment, the set of target probes, pre-amplifiers, amplifiers and label probes each comprise four or more subsets. In one embodiment, the target probe binding sites for the two or more subsets are intermingled on the target nucleic acid.

In one embodiment of a sample of the invention, each probe in each of the probe subsets comprises (a) a set of target probes, wherein the target probe set comprises a plurality of pairs of target probes that specifically hybridize to a target nucleic acid, wherein the target probes are hybridized to the target nucleic acid; (b) a set of pre-amplifiers, wherein the pre-amplifier set comprises a plurality of pre-amplifiers, wherein the pre-amplifiers comprise binding sites for the pairs of target probes and a plurality of binding sites for an amplifier, wherein the pre-amplifiers are hybridized to the target probes; (c) a set of amplifiers, wherein the amplifier set comprises a plurality of amplifiers, wherein the amplifiers comprise a binding site for the pre-amplifiers and a plurality of binding sites for a label probe or two or more distinct label probes, wherein the amplifiers are hybridized to the pre-amplifiers; and (d) a set of label probes, wherein the label probe set comprises a label probe or two or more distinct label probes, wherein each label probe comprises a label and a binding site for the amplifiers, wherein the labels in each distinct label probe are distinguishable between the distinct label probes, wherein the label probes are hybridized to the amplifiers; wherein the amplifier in each probe subset specific for a target nucleic acid comprises a binding site for a label or a combination of two or more distinct labels that is different for each probe subset.

In one embodiment of the sample, the label probe set comprises two or more distinct label probes, wherein the amplifier set comprises a plurality of non-identical amplifiers, and wherein the binding sites for the two or more distinct label probes on each non-identical amplifier are in a different order on each non-identical amplifier.

In one embodiment of a sample of the invention, each probe in each of the probe subsets comprises (a) a set of target probes, wherein the target probe set comprises a plurality of pairs of target probes that specifically hybridize to a target nucleic acid, wherein the target probes are hybridized to the target nucleic acid; (b) a set of pre-amplifiers, wherein the pre-amplifier set comprises a plurality of pre-amplifiers, wherein the pre-amplifiers comprise binding sites for the pairs of target probes and a plurality of binding sites for amplifiers, wherein the pre-amplifiers are hybridized to the target probes; (c) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for the pre-amplifiers and a plurality of binding sites for a distinct label probe, wherein the amplifiers are hybridized to the pre-amplifiers; and (d) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes, wherein the label probes are hybridized to the amplifiers; wherein the pre-amplifier in each probe subset specific for a target nucleic acid comprises a plurality of binding sites for the amplifier comprising a binding site for the label probe or a plurality of binding sites for the two or more distinct amplifiers comprising binding sites for the two or more distinct label probes, and wherein the label of the label probe or combination of two or more distinct labels of the two or more distinct label probes is different for each probe subset.

In one embodiment of the sample, the plurality of amplifiers comprise two or more distinct amplifiers, and wherein the binding sites on the pre-amplifier for the distinct amplifiers are intermingled.

In one embodiment of a sample of the invention, each probe in each of the probe subsets comprises (a) a set of target probes, wherein the target probe set comprises a plurality of pairs of target probes that specifically hybridize to a target nucleic acid, wherein the target probes are hybridized to the target nucleic acid; (b) a set of pre-amplifiers, wherein the pre-amplifier set comprises a plurality of pre-amplifiers, wherein the pre-amplifiers comprise binding sites for the pairs of target probes and a plurality of binding sites for an amplifier, wherein the pre-amplifiers are hybridized to the target probes; (c) a set of amplifiers, wherein the amplifier set comprises a plurality of amplifiers, wherein the amplifiers comprise a binding site for the pre-amplifiers and a plurality of identical binding sites for a label probe, wherein the amplifiers are hybridized to the pre-amplifiers; and (d) a set of label probes, wherein the label probe set comprises a label probe or two or more distinct label probes, wherein each label probe comprises a label and a binding site for the amplifiers, wherein the binding site for the amplifier is the same for each of the label probes, wherein the labels in each distinct label probe are distinguishable between the distinct label probes; wherein the amplifier in each probe subset specific for a target nucleic acid comprises a binding site for a label or a combination of two or more distinct labels that is different for each probe subset.

In one embodiment of the sample, the distinct labels of the two or more distinct label probes are the same in two probe subsets for two target nucleic acids and wherein the ratio of label probes bound to one target nucleic acid is different than the ratio of label probes bound to the second target nucleic acid, wherein a difference in ratios of distinct label probes in the first probe subset and the second probe subset distinguish the two target nucleic acids.

In one embodiment of a sample of the invention, each probe in each of the probe subsets comprises (a) a set of target probes, wherein the target probe set comprises a plurality of pairs of target probes that specifically hybridize to a target nucleic acid, wherein the target probes are hybridized to the target nucleic acid; (b) a set of pre-amplifiers, wherein the pre-amplifier set comprises a plurality of pre-amplifiers, wherein the pre-amplifiers comprise binding sites for the pairs of target probes and a plurality of binding sites for an amplifier, wherein the pre-amplifiers are hybridized to the target probes; (c) a set of amplifiers, wherein the amplifier set comprises a plurality of amplifiers, wherein the amplifiers comprise a binding site for the pre-amplifiers and a plurality of identical binding sites for a label probe, wherein the amplifiers are hybridized to the pre-amplifiers; and (d) a set of label probes, wherein the label probe set comprises a label probe or two or more distinct label probes, wherein each label probe comprises a label and a binding site for the amplifiers, wherein the binding site for the amplifiers is the same for each label probe, wherein the labels in each distinct label probe are distinguishable between the distinct label probes, wherein the label probes are hybridized to the amplifiers; wherein the amplifier in each probe subset specific for a target nucleic acid comprises a binding site for a label probe or a combination of two or more distinct label probes that is different for each probe subset.

In one embodiment of a sample of the invention, each probe in each of the probe subsets comprises (a) a set of target probes, wherein the target probe set comprises one or more subsets of target probes, wherein each target probe subset comprises a plurality of pairs of target probes that specifically hybridize to a target nucleic acid, wherein the target probes of a subset are hybridized to the target nucleic acid; (b) a set of pre-pre-amplifiers, wherein the pre-pre-amplifier set comprises one or more subsets of pre-pre-amplifiers, wherein the one or more pre-pre-amplifier subsets comprise a pre-pre-amplifier specific for each of the target probe pairs in the one or more target probe subsets, wherein each pre-pre-amplifier comprises binding sites for the pair of target probes of one of the target probe subsets and a plurality of binding sites for a pre-amplifier, wherein the pre-pre-amplifiers of a subset are hybridized to the respective target probe subset; (c) a set of pre-amplifiers, wherein the pre-amplifier set comprises one or more subsets of pre-amplifiers, wherein the one or more pre-amplifier subsets comprise a pre-amplifier specific for the pre-pre-amplifiers in the one or more pre-pre-amplifier subsets, wherein each pre-amplifier comprises binding sites for the pre-pre-amplifiers of one of the pre-pre-amplifier subsets and a plurality of binding sites for an amplifier, wherein the pre-amplifiers of a subset are hybridized to the respective pre-pre-amplifier subset; (d) a set of amplifiers, wherein the amplifier set comprises one or more subsets of amplifiers specific for each pre-amplifier subset, wherein each amplifier subset comprises a plurality of amplifiers, wherein the amplifiers of one of the amplifier subsets comprise a binding site for the pre-amplifiers of one of the pre-amplifier subsets and a plurality of binding sites for a label probe, wherein the amplifiers of a subset are hybridized to the respective pre-amplifier subset; and (e) a set of label probes, wherein the label probe set comprises one or more subsets of label probes, wherein each label probe subset is specific for one of the amplifier subsets, wherein each label probe subset comprises a plurality of label probes, wherein the label probes in each of the label probe subsets comprise a label and a binding site for the amplifiers of one of the amplifier subsets, wherein the labels in each label probe subset are distinguishable between the label probe subsets, wherein the label probes of a subset are hybridized to the respective amplifier subset; wherein the one or more label probe subsets in each probe subset specific for a target nucleic acid comprise at least one label or a combination of labels that is different for each probe subset.

In one embodiment of the sample, the set of target probes, pre-amplifiers, amplifiers and label probes each comprise two or more subsets. In another embodiment, the set of target probes, pre-amplifiers, amplifiers and label probes each comprise three or more subsets. In another embodiment, the set of target probes, pre-amplifiers, amplifiers and label probes each comprise four or more subsets. In one embodiment of the sample, the target probe binding sites for the two or more subsets are intermingled on the target nucleic acid.

In one embodiment of a sample of the invention, each probe in each of the probe subsets comprises (a) a set of target probes, wherein the target probe set comprises a plurality of pairs of target probes that specifically hybridize to a target nucleic acid, wherein the target probes are hybridized to the target nucleic acid; (b) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier, wherein the pre-pre-amplifiers are hybridized to the target probes; (c) a set of pre-amplifiers, wherein the pre-amplifier set comprises a plurality of pre-amplifiers, wherein the pre-amplifiers comprise binding sites for the pre-pre-amplifiers and a plurality of binding sites for an amplifier, wherein the pre-amplifiers are hybridized to the pre-pre-amplifiers; (d) a set of amplifiers, wherein the amplifier set comprises a plurality of amplifiers, wherein the amplifiers comprise a binding site for the pre-amplifiers and a plurality of binding sites for a label probe or two or more distinct label probes, wherein the amplifiers are hybridized to the pre-amplifiers; and (e) a set of label probes, wherein the label probe set comprises a label probe or two or more distinct label probes, wherein each label probe comprises a label and a binding site for the amplifiers, wherein the labels in each distinct label probe are distinguishable between the distinct label probes, wherein the label probes are hybridized to the amplifiers; wherein the amplifier in each probe subset specific for a target nucleic acid comprises a binding site for a label or a combination of two or more distinct labels that is different for each probe subset.

In one embodiment of the sample, the label probe set comprises two or more distinct label probes, wherein the amplifier set comprises a plurality of non-identical amplifiers, and wherein the binding sites for the two or more distinct label probes on each non-identical amplifier are in a different order on each non-identical amplifier.

In one embodiment of a sample of the invention, each probe in each of the probe subsets comprises (a) a set of target probes, wherein the target probe set comprises a plurality of pairs of target probes that specifically hybridize to a target nucleic acid, wherein the target probes are hybridized to the target nucleic acid; (b) a set of pre-pre-amplifiers, wherein the pre-pre-amplifier set comprises a plurality of pre-pre-amplifiers, wherein each pre-pre-amplifier comprises binding sites for the pairs of target probes and a plurality of binding sites for a pre-amplifier, wherein the pre-pre-amplifiers are hybridized to the target probes; (c) a set of pre-amplifiers, wherein the pre-amplifier set comprises a plurality of pre-amplifiers, wherein the pre-amplifiers comprise binding sites for the pre-pre-amplifiers and a plurality of binding sites for amplifiers, wherein the pre-amplifiers are hybridized to the pre-pre-amplifiers; (d) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for the pre-amplifiers and a plurality of binding sites for a distinct label probe, wherein the amplifiers are hybridized to the pre-amplifiers; and (e) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes, wherein the label probes are hybridized to the amplifiers; wherein the pre-amplifier in each probe subset specific for a target nucleic acid comprises a plurality of binding sites for the amplifier comprising a binding site for the label probe or a plurality of binding sites for the two or more distinct amplifiers comprising binding sites for the two or more distinct label probes, and wherein the label of the label probe or combination of two or more distinct labels of the two or more distinct label probes is different for each probe subset.

In one embodiment of the sample, the plurality of amplifiers comprise two or more distinct amplifiers, and wherein the binding sites on the pre-amplifier for the distinct amplifiers are intermingled.

In one embodiment of a sample of the invention, each probe in each of the probe subsets comprises (a) a set of target probes, wherein the target probe set comprises a plurality of pairs of target probes that specifically hybridize to a target nucleic acid, wherein the target probes are hybridized to the target nucleic acid; (b) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier or for two or more distinct pre-amplifiers, wherein the pre-pre-amplifiers are hybridized to the target probes; (c) a set of pre-amplifiers, wherein the pre-amplifier set comprises a plurality of pre-amplifiers, wherein the plurality of pre-amplifiers comprise a pre-amplifier comprising a binding site for the pre-pre-amplifiers and a plurality of binding sites for an amplifier, or wherein the plurality of pre-amplifiers comprise two or more distinct pre-amplifiers, wherein each distinct pre-amplifier comprises a binding site for the pre-pre-amplifiers and a plurality of binding sites for a distinct amplifier, wherein the pre-amplifiers are hybridized to the pre-pre-amplifiers; (d) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for one of the distinct pre-amplifiers and a plurality of binding sites for a distinct label probe, wherein the amplifiers are hybridized to the pre-amplifiers; and (e) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes, wherein the label probes are hybridized to the amplifiers; wherein the pre-pre-amplifier in each probe subset specific for a target nucleic acid comprises a plurality of binding sites for the pre-amplifier comprising a plurality of binding sites for the amplifier comprising a binding site for the label probe, or a plurality of binding sites for the two or more distinct pre-amplifiers each comprising a plurality of binding sites for one of the two or more distinct amplifiers comprising binding sites for one of the two or more distinct label probes, and wherein the label of the label probe or combination of two or more distinct labels of the two or more distinct label probes is different for each probe subset.

In one embodiment of the sample, the plurality of pre-amplifiers comprise two or more distinct pre-amplifiers, and wherein the binding sites on the pre-pre-amplifier for the distinct pre-amplifiers are intermingled.

In one embodiment of a sample of the invention, each probe in each of the probe subsets comprises (a) a set of target probes, wherein the target probe set comprises a plurality of pairs of target probes that specifically hybridize to a target nucleic acid; (b) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier; (c) a set of pre-amplifiers, wherein the pre-amplifier set comprises a plurality of pre-amplifiers, wherein the pre-amplifiers comprise binding sites for the pairs of target probes and a plurality of binding sites for an amplifier; (d) a set of amplifiers, wherein the amplifier set comprises a plurality of amplifiers, wherein the amplifiers comprise a binding site for the pre-amplifiers and a plurality of identical binding sites for a label probe; and (e) a set of label probes, wherein the label probe set comprises a label probe or two or more distinct label probes, wherein each label probe comprises a label and a binding site for the amplifiers, wherein the binding site for the amplifier is the same for each label probe, wherein the labels in each distinct label probe are distinguishable between the distinct label probes; wherein the amplifier in each probe subset specific for a target nucleic acid comprises a binding site for a label probe or a combination of two or more distinct label probes that is different for each probe subset.

In one embodiment of the sample, the distinct labels of the two or more distinct label probes are the same in two probe subsets for two target nucleic acids and wherein the ratio of label probes in one probe subset is different than the ratio of label probes in the second probe subset, wherein a difference in ratios of distinct label probes in the first probe subset and the second probe subset distinguish the two target nucleic acids.

The invention additionally provides a slide comprising a cell or a plurality of cells. Optionally, the cell or cells are fixed to the slide. Optionally, the cell or cells are permeabilized. In particular embodiments, the cells on the slide are fixed and/or permeabilized for an in situ assay.

In one embodiment, the invention provides a slide comprising (A) a slide having immobilized thereon at least one cell containing a plurality of target nucleic acids; and (B) set of probes, wherein the set of probes comprises subsets of probes comprising a plurality detectable labels that provide unique labeling of each target nucleic acid, wherein each probe subset comprises one or more distinct labels, wherein the number and/or combination of distinct labels is unique for each target nucleic acid, and wherein at least one subset of probes is specifically hybridized to a target nucleic acid.

In one embodiment of a slide of the invention, each probe in each of the probe subsets comprises (a) a set of target probes, wherein the target probe set comprises one or more subsets of target probes, wherein each target probe subset comprises a plurality of pairs of target probes that specifically hybridize to a target nucleic acid, wherein the target probes of a subset are hybridized to the target nucleic acid; (b) a set of pre-amplifiers, wherein the pre-amplifier set comprises one or more subsets of pre-amplifiers, wherein the one or more pre-amplifier subsets comprise a pre-amplifier specific for each of the target probe pairs in the one or more target probe subsets, wherein each pre-amplifier comprises binding sites for the pair of target probes of one of the target probe subsets and a plurality of binding sites for an amplifier, wherein the pre-amplifiers of a subset are hybridized to the respective target probe subset; (c) a set of amplifiers, wherein the amplifier set comprises one or more subsets of amplifiers specific for each pre-amplifier subset, wherein each amplifier subset comprises a plurality of amplifiers, wherein the amplifiers of one of the amplifier subsets comprise a binding site for the pre-amplifiers of one of the pre-amplifier subsets and a plurality of binding sites for a label probe, wherein the amplifiers of a subset are hybridized to the respective pre-amplifier subset; and (d) a set of label probes, wherein the label probe set comprises one or more subsets of label probes, wherein each label probe subset is specific for one of the amplifier subsets, wherein each label probe subset comprises a plurality of label probes, wherein the label probes in each of the label probe subsets comprise a label and a binding site for the amplifiers of one of the amplifier subsets, wherein the labels in each label probe subset are distinguishable between the label probe subsets, wherein the label probes of a subset are hybridized to the respective amplifier subset; wherein the one or more label probe subsets in each probe subset specific for a target nucleic acid comprise at least one label or a combination of labels that is different for each probe subset.

In one embodiment of the slide, the set of target probes, pre-amplifiers, amplifiers and label probes each comprise two or more subsets. In another embodiment, the set of target probes, pre-amplifiers, amplifiers and label probes each comprise three or more subsets. In another embodiment, the set of target probes, pre-amplifiers, amplifiers and label probes each comprise four or more subsets. In one embodiment of the slide, the target probe binding sites for the two or more subsets are intermingled on the target nucleic acid.

In one embodiment of a slide of the invention, each probe in each of the probe subsets comprises (a) a set of target probes, wherein the target probe set comprises a plurality of pairs of target probes that specifically hybridize to a target nucleic acid, wherein the target probes are hybridized to the target nucleic acid; (b) a set of pre-amplifiers, wherein the pre-amplifier set comprises a plurality of pre-amplifiers, wherein the pre-amplifiers comprise binding sites for the pairs of target probes and a plurality of binding sites for an amplifier, wherein the pre-amplifiers are hybridized to the target probes; (c) a set of amplifiers, wherein the amplifier set comprises a plurality of amplifiers, wherein the amplifiers comprise a binding site for the pre-amplifiers and a plurality of binding sites for a label probe or two or more distinct label probes, wherein the amplifiers are hybridized to the pre-amplifiers; and (d) a set of label probes, wherein the label probe set comprises a label probe or two or more distinct label probes, wherein each label probe comprises a label and a binding site for the amplifiers, wherein the labels in each distinct label probe are distinguishable between the distinct label probes, wherein the label probes are hybridized to the amplifiers; wherein the amplifier in each probe subset specific for a target nucleic acid comprises a binding site for a label or a combination of two or more distinct labels that is different for each probe subset.

In one embodiment of the slide, the label probe set comprises two or more distinct label probes, wherein the amplifier set comprises a plurality of non-identical amplifiers, and wherein the binding sites for the two or more distinct label probes on each non-identical amplifier are in a different order on each non-identical amplifier.

In one embodiment of a slide of the invention, each probe in each of the probe subsets comprises (a) a set of target probes, wherein the target probe set comprises a plurality of pairs of target probes that specifically hybridize to a target nucleic acid, wherein the target probes are hybridized to the target nucleic acid; (b) a set of pre-amplifiers, wherein the pre-amplifier set comprises a plurality of pre-amplifiers, wherein the pre-amplifiers comprise binding sites for the pairs of target probes and a plurality of binding sites for amplifiers, wherein the pre-amplifiers are hybridized to the target probes; (c) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for the pre-amplifiers and a plurality of binding sites for a distinct label probe, wherein the amplifiers are hybridized to the pre-amplifiers; and (d) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes, wherein the label probes are hybridized to the amplifiers; wherein the pre-amplifier in each probe subset specific for a target nucleic acid comprises a plurality of binding sites for the amplifier comprising a binding site for the label probe or a plurality of binding sites for the two or more distinct amplifiers comprising binding sites for the two or more distinct label probes, and wherein the label of the label probe or combination of two or more distinct labels of the two or more distinct label probes is different for each probe subset.

In one embodiment of the slide, the plurality of amplifiers comprise two or more distinct amplifiers, and wherein the binding sites on the pre-amplifier for the distinct amplifiers are intermingled.

In one embodiment of a slide of the invention, each probe in each of the probe subsets comprises (a) a set of target probes, wherein the target probe set comprises a plurality of pairs of target probes that specifically hybridize to a target nucleic acid, wherein the target probes are hybridized to the target nucleic acid; (b) a set of pre-amplifiers, wherein the pre-amplifier set comprises a plurality of pre-amplifiers, wherein the pre-amplifiers comprise binding sites for the pairs of target probes and a plurality of binding sites for an amplifier, wherein the pre-amplifiers are hybridized to the target probes; (c) a set of amplifiers, wherein the amplifier set comprises a plurality of amplifiers, wherein the amplifiers comprise a binding site for the pre-amplifiers and a plurality of identical binding sites for a label probe, wherein the amplifiers are hybridized to the pre-amplifiers; and (d) a set of label probes, wherein the label probe set comprises a label probe or two or more distinct label probes, wherein each label probe comprises a label and a binding site for the amplifiers, wherein the binding site for the amplifier is the same for each label probe, wherein the labels in each distinct label probe are distinguishable between the distinct label probes, wherein the label probes are hybridized to the amplifiers; wherein the amplifier in each probe subset specific for a target nucleic acid comprises a binding site for a label probe or a combination of two or more distinct label probes that is different for each probe subset.

In one embodiment of the slide, the distinct labels of the two or more distinct label probes are the same in two probe subsets for two target nucleic acids and wherein the ratio of label probes bound to one target nucleic acid is different than the ratio of label probes bound to the second target nucleic acid, wherein a difference in ratios of distinct label probes in the first probe subset and the second probe subset distinguish the two target nucleic acids.

In one embodiment of a slide of the invention, each probe in each of the probe subsets comprises (a) a set of target probes, wherein the target probe set comprises a plurality of pairs of target probes that specifically hybridize to a target nucleic acid, wherein the target probes are hybridized to the target nucleic acid; (b) a set of pre-amplifiers, wherein the pre-amplifier set comprises a plurality of pre-amplifiers, wherein the pre-amplifiers comprise binding sites for the pairs of target probes and a plurality of binding sites for an amplifier, wherein the pre-amplifiers are hybridized to the target probes; (c) a set of amplifiers, wherein the amplifier set comprises a plurality of amplifiers, wherein the amplifiers comprise a binding site for the pre-amplifiers and a plurality of identical binding sites for a label probe, wherein the amplifiers are hybridized to the pre-amplifiers; and (d) a set of label probes, wherein the label probe set comprises a label probe or two more distinct label probes, wherein each label probe comprises a label and a binding site for the amplifiers, wherein the binding site for the amplifier is the same for each label probe, wherein the labels in each distinct label probe are distinguishable between the distinct label probes, wherein the label probes are hybridized to the amplifiers; wherein the amplifier in each probe subset specific for a target nucleic acid comprises a binding site for a label probe or a combination of two or more distinct label probes that is different for each probe subset.

In one embodiment of the slide, the distinct labels of the two or more distinct label probes are the same in two probe subsets for two target nucleic acids and wherein the ratio of label probes in one probe subset is different than the ratio of label probes in the second probe subset, wherein a difference in ratios of distinct label probes in the first probe subset and the second probe subset distinguish the two target nucleic acids.

In one embodiment of a slide of the invention, each probe in each of the probe subsets comprises (a) a set of target probes, wherein the target probe set comprises one or more subsets of target probes, wherein each target probe subset comprises a plurality of pairs of target probes that specifically hybridize to a target nucleic acid, wherein the target probes of a subset are hybridized to the target nucleic acid; (b) a set of pre-pre-amplifiers, wherein the pre-pre-amplifier set comprises one or more subsets of pre-pre-amplifiers, wherein the one or more pre-pre-amplifier subsets comprise a pre-pre-amplifier specific for each of the target probe pairs in the one or more target probe subsets, wherein each pre-pre-amplifier comprises binding sites for the pair of target probes of one of the target probe subsets and a plurality of binding sites for a pre-amplifier, wherein the pre-pre-amplifiers of a subset are hybridized to the respective target probe subset; (c) a set of pre-amplifiers, wherein the pre-amplifier set comprises one or more subsets of pre-amplifiers, wherein the one or more pre-amplifier subsets comprise a pre-amplifier specific for the pre-pre-amplifiers in the one or more pre-pre-amplifier subsets, wherein each pre-amplifier comprises binding sites for the pre-pre-amplifiers of one of the pre-pre-amplifier subsets and a plurality of binding sites for an amplifier, wherein the pre-amplifiers of a subset are hybridized to the respective pre-pre-amplifier subset; (d) a set of amplifiers, wherein the amplifier set comprises one or more subsets of amplifiers specific for each pre-amplifier subset, wherein each amplifier subset comprises a plurality of amplifiers, wherein the amplifiers of one of the amplifier subsets comprise a binding site for the pre-amplifiers of one of the pre-amplifier subsets and a plurality of binding sites for a label probe, wherein the amplifiers of a subset are hybridized to the respective pre-amplifier subset; and (e) a set of label probes, wherein the label probe set comprises one or more subsets of label probes, wherein each label probe subset is specific for one of the amplifier subsets, wherein each label probe subset comprises a plurality of label probes, wherein the label probes in each of the label probe subsets comprise a label and a binding site for the amplifiers of one of the amplifier subsets, wherein the labels in each label probe subset are distinguishable between the label probe subsets, wherein the label probes of a subset are hybridized to the respective amplifier subset; wherein the one or more label probe subsets in each probe subset specific for a target nucleic acid comprise at least one label or a combination of labels that is different for each probe subset.

In one embodiment of the slide, the set of target probes, pre-amplifiers, amplifiers and label probes each comprise two or more subsets. In another embodiment, the set of target probes, pre-amplifiers, amplifiers and label probes each comprise three or more subsets. In another embodiment, the set of target probes, pre-amplifiers, amplifiers and label probes each comprise four or more subsets. In another embodiment of the slide, the target probe binding sites for the two or more subsets are intermingled on the target nucleic acid.

In one embodiment of a slide of the invention, each probe in each of the probe subsets comprises (a) a set of target probes, wherein the target probe set comprises a plurality of pairs of target probes that specifically hybridize to a target nucleic acid, wherein the target probes are hybridized to the target nucleic acid; (b) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier, wherein the pre-pre-amplifiers are hybridized to the target probes; (c) a set of pre-amplifiers, wherein the pre-amplifier set comprises a plurality of pre-amplifiers, wherein the pre-amplifiers comprise binding sites for the pre-pre-amplifiers and a plurality of binding sites for an amplifier, wherein the pre-amplifiers are hybridized to the pre-pre-amplifiers; (d) a set of amplifiers, wherein the amplifier set comprises a plurality of amplifiers, wherein the amplifiers comprise a binding site for the pre-amplifiers and a plurality of binding sites for a label probe or two or more distinct label probes, wherein the amplifiers are hybridized to the pre-amplifiers; and (e) a set of label probes, wherein the label probe set comprises a label probe or two or more distinct label probes, wherein each label probe comprises a label and a binding site for the amplifiers, wherein the labels in each distinct label probe are distinguishable between the distinct label probes, wherein the label probes are hybridized to the amplifiers; wherein the amplifier in each probe subset specific for a target nucleic acid comprises a binding site for a label or a combination of two or more distinct labels that is different for each probe subset.

In one embodiment of the slide, the label probe set comprises two or more distinct label probes, wherein the amplifier set comprises a plurality of non-identical amplifiers, and wherein the binding sites for the two or more distinct label probes on each non-identical amplifier are in a different order on each non-identical amplifier.

In one embodiment of a slide of the invention, each probe in each of the probe subsets comprises (a) a set of target probes, wherein the target probe set comprises a plurality of pairs of target probes that specifically hybridize to a target nucleic acid, wherein the target probes are hybridized to the target nucleic acid; (b) a set of pre-pre-amplifiers, wherein the pre-pre-amplifier set comprises a plurality of pre-pre-amplifiers, wherein each pre-pre-amplifier comprises binding sites for the pairs of target probes and a plurality of binding sites for a pre-amplifier, wherein the pre-pre-amplifiers are hybridized to the target probes; (c) a set of pre-amplifiers, wherein the pre-amplifier set comprises a plurality of pre-amplifiers, wherein the pre-amplifiers comprise binding sites for the pre-pre-amplifiers and a plurality of binding sites for amplifiers, wherein the pre-amplifiers are hybridized to the pre-pre-amplifiers; (d) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for the pre-amplifiers and a plurality of binding sites for a distinct label probe, wherein the amplifiers are hybridized to the pre-amplifiers; and (e) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes, wherein the label probes are hybridized to the amplifiers; wherein the pre-amplifier in each probe subset specific for a target nucleic acid comprises a plurality of binding sites for the amplifier comprising a binding site for the label probe or a plurality of binding sites for the two or more distinct amplifiers comprising binding sites for the two or more distinct label probes, and wherein the label of the label probe or combination of two or more distinct labels of the two or more distinct label probes is different for each probe subset.

In one embodiment of the slide, the plurality of amplifiers comprise two or more distinct amplifiers, and wherein the binding sites on the pre-amplifier for the distinct amplifiers are intermingled.

In one embodiment of a slide of the invention, each probe in each of the probe subsets comprises (a) a set of target probes, wherein the target probe set comprises a plurality of pairs of target probes that specifically hybridize to a target nucleic acid, wherein the target probes are hybridized to the target nucleic acid; (b) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier or for two or more distinct pre-amplifiers, wherein the pre-pre-amplifiers are hybridized to the target probes; (c) a set of pre-amplifiers, wherein the pre-amplifier set comprises a plurality of pre-amplifiers, wherein the plurality of pre-amplifiers comprise a pre-amplifier comprising a binding site for the pre-pre-amplifiers and a plurality of binding sites for an amplifier, or wherein the plurality of pre-amplifiers comprise two or more distinct pre-amplifiers, wherein each distinct pre-amplifier comprises a binding site for the pre-pre-amplifiers and a plurality of binding sites for a distinct amplifier, wherein the pre-amplifiers are hybridized to the pre-pre-amplifiers; (d) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for one of the distinct pre-amplifiers and a plurality of binding sites for a distinct label probe, wherein the amplifiers are hybridized to the pre-amplifiers; and (e) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes, wherein the label probes are hybridized to the amplifiers; wherein the pre-pre-amplifier in each probe subset specific for a target nucleic acid comprises a plurality of binding sites for the pre-amplifier comprising a plurality of binding sites for the amplifier comprising a binding site for the label probe, or a plurality of binding sites for the two or more distinct pre-amplifiers each comprising a plurality of binding sites for one of the two or more distinct amplifiers comprising binding sites for one of the two or more distinct label probes, and wherein the label of the label probe or combination of two or more distinct labels of the two or more distinct label probes is different for each probe subset.

In one embodiment of the slide, the plurality of pre-amplifiers comprise two or more distinct pre-amplifiers, and wherein the binding sites on the pre-pre-amplifier for the distinct pre-amplifiers are intermingled.

In one embodiment of a slide of the invention, each probe in each of the probe subsets comprises (a) a set of target probes, wherein the target probe set comprises a plurality of pairs of target probes that specifically hybridize to a target nucleic acid; (b) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier; (c) a set of pre-amplifiers, wherein the pre-amplifier set comprises a plurality of pre-amplifiers, wherein the pre-amplifiers comprise binding sites for the pairs of target probes and a plurality of binding sites for an amplifier; (d) a set of amplifiers, wherein the amplifier set comprises a plurality of amplifiers, wherein the amplifiers comprise a binding site for the pre-amplifiers and a plurality of identical binding sites for a label probe; and (e) a set of label probes, wherein the label probe set comprises a label probe or two or more distinct label probes, wherein each label probe comprises a label and a binding site for the amplifiers, wherein the binding site for the amplifier is the same for each label probe, wherein the labels in each distinct label probe are distinguishable between the distinct label probes; wherein the amplifier in each probe subset specific for a target nucleic acid comprises a binding site for a label probe or a combination of two or more distinct label probes that is different for each probe subset.

In one embodiment of the slide, the distinct labels of the two or more distinct label probes are the same in two probe subsets for two target nucleic acids and wherein the ratio of label probes in one probe subset is different than the ratio of label probes in the second probe subset, wherein a difference in ratios of distinct label probes in the first probe subset and the second probe subset distinguish the two target nucleic acids.

The invention also provides a kit comprising the components of an SGC, as described herein, for multiplex labeling of target nucleic acids. The components of a kit of the invention can optionally be in a container, and optionally instructions for using the kit can be provided. Optionally, the kit can comprise one or more components of an SGC, as described herein, where the kit does not include the target nucleic acid. Such a kit can comprise pre-amplifiers (PAs), amplifiers (AMPs) and label probes (LPs), and optionally pre-pre-amplifiers (PPAs), as disclosed herein. Optionally the kit can comprise target probes (TPs) directed to a particular target nucleic acid, or a plurality of target nucleic acids.

In one embodiment, the invention provides a kit for multiplex detection of a plurality of target nucleic acids in a cell, comprising a set of probes, wherein the set of probes comprises subsets of probes comprising a plurality detectable labels that provide unique labeling of each target nucleic acid, wherein each probe subset comprises one or more distinct labels, wherein the number and/or combination of distinct labels is unique for each target nucleic acid.

In one embodiment of a kit of the invention, each probe in each of the probe subsets comprises (a) a set of pre-amplifiers, wherein the pre-amplifier set comprises one or more subsets of pre-amplifiers, wherein the one or more pre-amplifier subsets comprise a pre-amplifier specific for each of the target probe pairs in the one or more target probe subsets, wherein each pre-amplifier comprises binding sites for the pair of target probes of one of the target probe subsets and a plurality of binding sites for an amplifier; (b) a set of amplifiers, wherein the amplifier set comprises one or more subsets of amplifiers specific for each pre-amplifier subset, wherein each amplifier subset comprises a plurality of amplifiers, wherein the amplifiers of one of the amplifier subsets comprise a binding site for the pre-amplifiers of one of the pre-amplifier subsets and a plurality of binding sites for a label probe; and (c) a set of label probes, wherein the label probe set comprises one or more subsets of label probes, wherein each label probe subset is specific for one of the amplifier subsets, wherein each label probe subset comprises a plurality of label probes, wherein the label probes in each of the label probe subsets comprise a label and a binding site for the amplifiers of one of the amplifier subsets, wherein the labels in each label probe subset are distinguishable between the label probe subsets; wherein the one or more label probe subsets in each probe subset specific for a target nucleic acid comprise at least one label or a combination of labels that is different for each probe subset.

In one embodiment, the kit comprises a set of target probes, wherein the target probe set comprises one or more subsets of target probes, wherein each target probe subset comprises a plurality of pairs of target probes that specifically hybridize to a target nucleic acid. In one embodiment of the kit, the set of target probes, pre-amplifiers, amplifiers and label probes each comprise two or more subsets. In another embodiment, the set of target probes, pre-amplifiers, amplifiers and label probes each comprise three or more subsets. In another embodiment, the set of target probes, pre-amplifiers, amplifiers and label probes each comprise four or more subsets. In one embodiment of the kit, the target probe binding sites for the two or more subsets are intermingled on the target nucleic acid.

In one embodiment of a kit of the invention, each probe in each of the probe subsets comprises (a) a set of pre-amplifiers, wherein the pre-amplifier set comprises a plurality of pre-amplifiers, wherein the pre-amplifiers comprise binding sites for the pairs of target probes and a plurality of binding sites for an amplifier; (b) a set of amplifiers, wherein the amplifier set comprises a plurality of amplifiers, wherein the amplifiers comprise a binding site for the pre-amplifiers and a plurality of binding sites for a label probe or two or more distinct label probes; and (c) a set of label probes, wherein the label probe set comprises a label probe or two or more distinct label probes, wherein each label probe comprises a label and a binding site for the amplifiers, wherein the labels in each distinct label probe are distinguishable between the distinct label probes; wherein the amplifier in each probe subset specific for a target nucleic acid comprises a binding site for a label or a combination of two or more distinct labels that is different for each probe subset.

In one embodiment, the kit comprises a set of target probes, wherein the target probe set comprises a plurality of pairs of target probes that specifically hybridize to a target nucleic acid. In one embodiment of the kit, the label probe set comprises two or more distinct label probes, wherein the amplifier set comprises a plurality of non-identical amplifiers, and wherein the binding sites for the two or more distinct label probes on each non-identical amplifier are in a different order on each non-identical amplifier.

In one embodiment of a kit of the invention, each probe in each of the probe subsets comprises (a) a set of pre-amplifiers, wherein the pre-amplifier set comprises a plurality of pre-amplifiers, wherein the pre-amplifiers comprise binding sites for the pairs of target probes and a plurality of binding sites for amplifiers; (b) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for the pre-amplifiers and a plurality of binding sites for a distinct label probe; and (c) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes; wherein the pre-amplifier in each probe subset specific for a target nucleic acid comprises a plurality of binding sites for the amplifier comprising a binding site for the label probe or a plurality of binding sites for the two or more distinct amplifiers comprising binding sites for the two or more distinct label probes, and wherein the label of the label probe or combination of two or more distinct labels of the two or more distinct label probes is different for each probe subset.

In one embodiment, the kit comprises a set of target probes, wherein the target probe set comprises a plurality of pairs of target probes that specifically hybridize to a target nucleic acid. In one embodiment of the kit, the plurality of amplifiers comprise two or more distinct amplifiers, and wherein the binding sites on the pre-amplifier for the distinct amplifiers are intermingled.

In one embodiment of a kit of the invention, each probe in each of the probe subsets comprises (a) a set of pre-amplifiers, wherein the pre-amplifier set comprises a plurality of pre-amplifiers, wherein the pre-amplifiers comprise binding sites for the pairs of target probes and a plurality of binding sites for an amplifier; (b) a set of amplifiers, wherein the amplifier set comprises a plurality of amplifiers, wherein the amplifiers comprise a binding site for the pre-amplifiers and a plurality of identical binding sites for a label probe; and (c) a set of label probes, wherein the label probe set comprises a label probe or two or more distinct label probes, wherein each label probe comprises a label and a binding site for the amplifiers, wherein the binding site for the amplifier is the same for each label probe, wherein the labels in each distinct label probe are distinguishable between the distinct label probes; wherein the amplifier in each probe subset specific for a target nucleic acid comprises a binding site for a label probe or a combination of two or more distinct label probes that is different for each probe subset.

In one embodiment of the kit, the distinct labels of the two or more distinct label probes are the same in two probe subsets for two target nucleic acids and wherein the ratio of label probes in one probe subset is different than the ratio of label probes in the second probe subset, wherein a difference in ratios of distinct label probes in the first probe subset and the second probe subset distinguish the two target nucleic acids.

In one embodiment, the kit comprises a set of target probes, wherein the target probe set comprises a plurality of pairs of target probes that specifically hybridize to a target nucleic acid.

In one embodiment of a kit of the invention, each probe in each of the probe subsets comprises (a) a set of pre-amplifiers, wherein the pre-amplifier set comprises a plurality of pre-amplifiers, wherein the pre-amplifiers comprise binding sites for the pairs of target probes and a plurality of binding sites for an amplifier; (b) a set of amplifiers, wherein the amplifier set comprises a plurality of amplifiers, wherein the amplifiers comprise a binding site for the pre-amplifiers and a plurality of identical binding sites for a label probe; and (c) a set of label probes, wherein the label probe set comprises a label probe or two or more distinct label probes, wherein each label probe comprises a label and a binding site for the amplifiers, wherein the binding site for the amplifier is the same for each label probe, wherein the labels in each distinct label probe are distinguishable between the distinct label probes; and wherein the label in each label probe subset is distinct from the label in another label probe subset; wherein the amplifier in each probe subset specific for a target nucleic acid comprises a binding site for a label probe or a combination of two or more distinct label probes that is different for each probe subset.

In one embodiment of the kit, the distinct labels of the two or more distinct label probes are the same in two probe subsets for two target nucleic acids and wherein the ratio of label probes in one probe subset is different than the ratio of label probes in the second probe subset, wherein a difference in ratios of distinct label probes in the first probe subset and the second probe subset distinguish the two target nucleic acids.

In one embodiment of a kit of the invention, each probe in each of the probe subsets comprises (a) a set of pre-pre-amplifiers, wherein the pre-pre-amplifier set comprises one or more subsets of pre-pre-amplifiers, wherein the one or more pre-pre-amplifier subsets comprise a pre-pre-amplifier specific for each of the target probe pairs in the one or more target probe subsets, wherein each pre-pre-amplifier comprises binding sites for the pair of target probes of one of the target probe subsets and a plurality of binding sites for a pre-amplifier; (b) a set of pre-amplifiers, wherein the pre-amplifier set comprises one or more subsets of pre-amplifiers, wherein the one or more pre-amplifier subsets comprise a pre-amplifier specific for the pre-pre-amplifiers in the one or more pre-pre-amplifier subsets, wherein each pre-amplifier comprises binding sites for the pre-pre-amplifiers of one of the pre-pre-amplifier subsets and a plurality of binding sites for an amplifier; (c) a set of amplifiers, wherein the amplifier set comprises one or more subsets of amplifiers specific for each pre-amplifier subset, wherein each amplifier subset comprises a plurality of amplifiers, wherein the amplifiers of one of the amplifier subsets comprise a binding site for the pre-amplifiers of one of the pre-amplifier subsets and a plurality of binding sites for a label probe; and (d) a set of label probes, wherein the label probe set comprises one or more subsets of label probes, wherein each label probe subset is specific for one of the amplifier subsets, wherein each label probe subset comprises a plurality of label probes, wherein the label probes in each of the label probe subsets comprise a label and a binding site for the amplifiers of one of the amplifier subsets, wherein the labels in each label probe subset are distinguishable between the label probe subsets; wherein the one or more label probe subsets in each probe subset specific for a target nucleic acid comprise at least one label or a combination of labels that is different for each probe subset.

In one embodiment, the kit comprises a set of target probes, wherein the target probe set comprises one or more subsets of target probes, wherein each target probe subset comprises a plurality of pairs of target probes that specifically hybridize to a target nucleic acid. In one embodiment of the kit, the set of target probes, pre-amplifiers, amplifiers and label probes each comprise two or more subsets. In another embodiment, the set of target probes, pre-amplifiers, amplifiers and label probes each comprise three or more subsets. In another embodiment, the set of target probes, pre-amplifiers, amplifiers and label probes each comprise four or more subsets. In one embodiment of the kit, the target probe binding sites for the two or more subsets are intermingled on the target nucleic acid.

In one embodiment of a kit of the invention, each probe in each of the probe subsets comprises (a) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier; (b) a set of pre-amplifiers, wherein the pre-amplifier set comprises a plurality of pre-amplifiers, wherein the pre-amplifiers comprise binding sites for the pre-pre-amplifiers and a plurality of binding sites for an amplifier; (c) a set of amplifiers, wherein the amplifier set comprises a plurality of amplifiers, wherein the amplifiers comprise a binding site for the pre-amplifiers and a plurality of binding sites for a label probe or two or more distinct label probes; and (d) a set of label probes, wherein the label probe set comprises a label probe or two or more distinct label probes, wherein each label probe comprises a label and a binding site for the amplifiers, wherein the labels in each distinct label probe are distinguishable between the distinct label probes; wherein the amplifier in each probe subset specific for a target nucleic acid comprises a binding site for a label or a combination of two or more distinct labels that is different for each probe subset.

In one embodiment, the kit comprises a set of target probes, wherein the target probe set comprises a plurality of pairs of target probes that specifically hybridize to a target nucleic acid. In one embodiment of the kit, the label probe set comprises two or more distinct label probes, wherein the amplifier set comprises a plurality of non-identical amplifiers, and wherein the binding sites for the two or more distinct label probes on each non-identical amplifier are in a different order on each non-identical amplifier.

In one embodiment of a kit of the invention, each probe in each of the probe subsets comprises (a) a set of pre-pre-amplifiers, wherein the pre-pre-amplifier set comprises a plurality of pre-pre-amplifiers, wherein each pre-pre-amplifier comprises binding sites for the pairs of target probes and a plurality of binding sites for a pre-amplifier; (b) a set of pre-amplifiers, wherein the pre-amplifier set comprises a plurality of pre-amplifiers, wherein the pre-amplifiers comprise binding sites for the pre-pre-amplifiers and a plurality of binding sites for amplifiers; (c) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for the pre-amplifiers and a plurality of binding sites for a distinct label probe; and (d) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes; wherein the pre-amplifier in each probe subset specific for a target nucleic acid comprises a plurality of binding sites for the amplifier comprising a binding site for the label probe or a plurality of binding sites for the two or more distinct amplifiers comprising binding sites for the two or more distinct label probes, and wherein the label of the label probe or combination of two or more distinct labels of the two or more distinct label probes is different for each probe subset.

In one embodiment, the kit comprises a set of target probes, wherein the target probe set comprises a plurality of pairs of target probes that specifically hybridize to a target nucleic acid. In one embodiment of the kit, the plurality of amplifiers comprise two or more distinct amplifiers, and wherein the binding sites on the pre-amplifier for the distinct amplifiers are intermingled.

In one embodiment of a kit of the invention, each probe in each of the probe subsets comprises (a) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier or for two or more distinct pre-amplifiers; (b) a set of pre-amplifiers, wherein the pre-amplifier set comprises a plurality of pre-amplifiers, wherein the plurality of pre-amplifiers comprise a pre-amplifier comprising a binding site for the pre-pre-amplifiers and a plurality of binding sites for an amplifier, or wherein the plurality of pre-amplifiers comprise two or more distinct pre-amplifiers, wherein each distinct pre-amplifier comprises a binding site for the pre-pre-amplifiers and a plurality of binding sites for a distinct amplifier; (c) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for one of the distinct pre-amplifiers and a plurality of binding sites for a distinct label probe; and (d) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes; wherein the pre-pre-amplifier in each probe subset specific for a target nucleic acid comprises a plurality of binding sites for the pre-amplifier comprising a plurality of binding sites for the amplifier comprising a binding site for the label probe, or a plurality of binding sites for the two or more distinct pre-amplifiers each comprising a plurality of binding sites for one of the two or more distinct amplifiers comprising binding sites for one of the two or more distinct label probes, and wherein the label of the label probe or combination of two or more distinct labels of the two or more distinct label probes is different for each probe subset.

In one embodiment, the kit comprises a set of target probes, wherein the target probe set comprises a plurality of pairs of target probes that specifically hybridize to a target nucleic acid. In one embodiment of the kit, the plurality of pre-amplifiers comprise two or more distinct pre-amplifiers, and wherein the binding sites on the pre-pre-amplifier for the distinct pre-amplifiers are intermingled.

In one embodiment of a kit of the invention, each probe in each of the probe subsets comprises (a) a set of target probes, wherein the target probe set comprises a plurality of pairs of target probes that specifically hybridize to a target nucleic acid; (b) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier; (c) a set of pre-amplifiers, wherein the pre-amplifier set comprises a plurality of pre-amplifiers, wherein the pre-amplifiers comprise binding sites for the pairs of target probes and a plurality of binding sites for an amplifier; (d) a set of amplifiers, wherein the amplifier set comprises a plurality of amplifiers, wherein the amplifiers comprise a binding site for the pre-amplifiers and a plurality of identical binding sites for a label probe; and (e) a set of label probes, wherein the label probe set comprises a label probe or two or more distinct label probes, wherein each label probe comprises a label and a binding site for the amplifiers, wherein the binding site for the amplifier is the same for each label probe, wherein the labels in each distinct label probe are distinguishable between the distinct label probes; wherein the amplifier in each probe subset specific for a target nucleic acid comprises a binding site for a label probe or a combination of two or more distinct label probes that is different for each probe subset.

In one embodiment of the kit, the distinct labels of the two or more distinct label probes are the same in two probe subsets for two target nucleic acids and wherein the ratio of label probes in one probe subset is different than the ratio of label probes in the second probe subset, wherein a difference in ratios of distinct label probes in the first probe subset and the second probe subset distinguish the two target nucleic acids.

In one embodiment, the kit comprises a reagent for fixing and/or permeabilizing cells.

It is understood that modifications which do not substantially affect the activity of the various embodiments of this invention are also provided within the definition of the invention provided herein. Accordingly, the following examples are intended to illustrate but not limit the present invention.

### Example I

### Multiplex Detection of Target Nucleic Acids

This example describes multiplex detection of four target nucleic acids.

Three fluorescent dyes (Alexa488, ATTO550 and ATTO647N) were used to detect four target mRNAs, 5-hydroxytryptamine receptor 7 (Htr7), protocadherin 8 (Pcdh8), tyrosine hydroxylase (Th), and forkhead box P1 (Foxp1). The assay was performed on frozen mouse brain section using the RNAscope® HiPlex assay (acdbio.com/rnascope-hiplex-assays). The fluorescent code for each target was as follows: Htr7, 1000 (Alexa488), Pcdh8, 0100 (ATTO550), Th, 1100 (Alexa488, ATTO550) and Foxp1, 1010 (Alexa488, ATTO647N). The configuration of the assay is essentially as described in FIG. 4C.

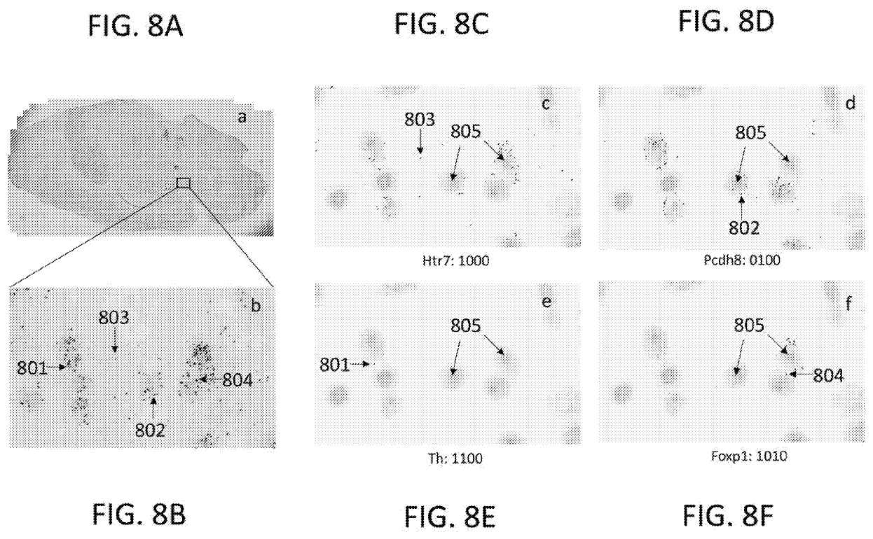

FIG. 8A shows an overview of stained mouse brain section. The boxed region in FIG. 8A is shown with 40× magnification in FIG. 8B. The zoomed image was processed with the Richardson-Lucy spatial deconvolution algorithm in MATLAB (Mathworks; Natick, Mass.), signal dots were detected (exemplary signal dots shown with arrows labeled 801-804), and colors were decoded to individual targets and shown in FIGS. 8C-8F. Nuclei were stained with DAPI (exemplary staining labeled 805).

These results demonstrate that three fluorescent “color” codes can be used to distinctly label and detect four target nucleic acids.

Throughout this application various publications have been referenced. The disclosures of these publications in their entireties are hereby incorporated by reference in this application in order to more fully describe the state of the art to which this invention pertains. Although the invention has been described with reference to the examples provided above, it should be understood that various modifications can be made without departing from the spirit of the invention.

What is claimed is:

1. A method for multiplex detection of a plurality of target nucleic acids in a cell, comprising:
(A) contacting a sample comprising a cell comprising a plurality of target nucleic acids with a set of probes, wherein the set of probes comprises subsets of probes comprising a plurality detectable labels that provide unique labeling of each target nucleic acid, wherein each probe subset comprises one or more distinct labels, wherein the number and/or combination of distinct labels is unique for each target nucleic acid; and
(B) detecting the detectable labels bound to the respective target nucleic acids.

2. The method of claim 1, wherein each probe in each of the probe subsets comprises:
(a) a set of target probes, wherein the target probe set comprises one or more subsets of target probes, wherein each target probe subset comprises a plurality of pairs of target probes that specifically hybridize to a target nucleic acid;
(b) a set of pre-amplifiers, wherein the pre-amplifier set comprises one or more subsets of pre-amplifiers, wherein the one or more pre-amplifier subsets comprise a pre-amplifier specific for each of the target probe pairs in the one or more target probe subsets, wherein each pre-amplifier comprises binding sites for the pair of target probes of one of the target probe subsets and a plurality of binding sites for an amplifier;
(c) a set of amplifiers, wherein the amplifier set comprises one or more subsets of amplifiers specific for each pre-amplifier subset, wherein each amplifier subset comprises a plurality of amplifiers, wherein the amplifiers of one of the amplifier subsets comprise a binding site for the pre-amplifiers of one of the pre-amplifier subsets and a plurality of binding sites for a label probe; and
(d) a set of label probes, wherein the label probe set comprises one or more subsets of label probes, wherein each label probe subset is specific for one of the amplifier subsets, wherein each label probe subset comprises a plurality of label probes, wherein the label probes in each of the label probe subsets comprise a label and a binding site for the amplifiers of one of the amplifier subsets, wherein the labels in each label probe subset are distinguishable between the label probe subsets;
wherein the one or more label probe subsets in each probe subset specific for a target nucleic acid comprise at least one label or a combination of labels that is different for each probe subset.

3. The method of claim 2, wherein the set of target probes, pre-amplifiers, amplifiers and label probes each comprise two or more subsets.

4. The method of claim 2, wherein the set of target probes, pre-amplifiers, amplifiers and label probes each comprise three or more subsets.

5. The method of claim 2, wherein the set of target probes, pre-amplifiers, amplifiers and label probes each comprise four or more subsets.

6. The method of claim 3, wherein the target probe binding sites for the two or more subsets are intermingled on the target nucleic acid.

7. The method of claim 1, wherein each probe in each of the probe subsets comprises:
(a) a set of target probes, wherein the target probe set comprises a plurality of pairs of target probes that specifically hybridize to a target nucleic acid;
(b) a set of pre-amplifiers, wherein the pre-amplifier set comprises a plurality of pre-amplifiers, wherein the pre-amplifiers comprise binding sites for the pairs of target probes and a plurality of binding sites for an amplifier;
(c) a set of amplifiers, wherein the amplifier set comprises a plurality of amplifiers, wherein the amplifiers comprise a binding site for the pre-amplifiers and a plurality of binding sites for a label probe or two or more distinct label probes; and
(d) a set of label probes, wherein the label probe set comprises a label probe or two or more distinct label probes, wherein each label probe comprises a label and a binding site for the amplifiers, wherein the labels in each distinct label probe are distinguishable between the distinct label probes;
wherein the amplifier in each probe subset specific for a target nucleic acid comprises a binding site for a label or a combination of two or more distinct labels that is different for each probe subset.

8. The method of claim 7, wherein the label probe set comprises two or more distinct label probes, wherein the amplifier set comprises a plurality of non-identical amplifiers, and wherein the binding sites for the two or more distinct label probes on each non-identical amplifier are in a different order on each non-identical amplifier.

9. The method of claim 1, wherein each probe in each of the probe subsets comprises:
(a) a set of target probes, wherein the target probe set comprises a plurality of pairs of target probes that specifically hybridize to a target nucleic acid;
(b) a set of pre-amplifiers, wherein the pre-amplifier set comprises a plurality of pre-amplifiers, wherein the pre-amplifiers comprise binding sites for the pairs of target probes and a plurality of binding sites for amplifiers;
(c) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for the pre-amplifiers and a plurality of binding sites for a distinct label probe; and
(d) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes;
wherein the pre-amplifier in each probe subset specific for a target nucleic acid comprises a plurality of binding sites for the amplifier comprising a binding site for the label probe or a plurality of binding sites for the two or more distinct amplifiers comprising binding sites for the two or more distinct label probes, and wherein the label of the label probe or combination of two or more distinct labels of the two or more distinct label probes is different for each probe subset.

10. The method of claim 9, wherein the plurality of amplifiers comprise two or more distinct amplifiers, and wherein the binding sites on the pre-amplifier for the distinct amplifiers are intermingled.

11. The method of claim 1, wherein each probe in each of the probe subsets comprises:
(a) a set of target probes, wherein the target probe set comprises a plurality of pairs of target probes that specifically hybridize to a target nucleic acid;
(b) a set of pre-amplifiers, wherein the pre-amplifier set comprises a plurality of pre-amplifiers, wherein the pre-amplifiers comprise binding sites for the pairs of target probes and a plurality of binding sites for an amplifier;
(c) a set of amplifiers, wherein the amplifier set comprises a plurality of amplifiers, wherein the amplifiers comprise a binding site for the pre-amplifiers and a plurality of identical binding sites for a label probe; and
(d) a set of label probes, wherein the label probe set comprises a label probe or two or more distinct label probes, wherein each label probe comprises a label and a binding site for the amplifiers, wherein the binding site for the amplifier is the same for each label probe, wherein the labels in each distinct label probe are distinguishable between the distinct label probes;
wherein the amplifier in each probe subset specific for a target nucleic acid comprises a binding site for a label probe or a combination of two or more distinct label probes that is different for each probe subset.

12. The method of claim 11, wherein the distinct labels of the two or more distinct label probes are the same in two probe subsets for two target nucleic acids and wherein the ratio of label probes in one probe subset is different than the ratio of label probes in the second probe subset, wherein a difference in ratios of distinct label probes in the first probe subset and the second probe subset distinguish the two target nucleic acids.

13. The method of claim 1, wherein each probe in each of the probe subsets comprises:
(a) a set of target probes, wherein the target probe set comprises one or more subsets of target probes, wherein each target probe subset comprises a plurality of pairs of target probes that specifically hybridize to a target nucleic acid;
(b) a set of pre-pre-amplifiers, wherein the pre-pre-amplifier set comprises one or more subsets of pre-pre-amplifiers, wherein the one or more pre-pre-amplifier subsets comprise a pre-pre-amplifier specific for each of the target probe pairs in the one or more target probe subsets, wherein each pre-pre-amplifier comprises binding sites for the pair of target probes of one of the target probe subsets and a plurality of binding sites for a pre-amplifier;
(c) a set of pre-amplifiers, wherein the pre-amplifier set comprises one or more subsets of pre-amplifiers, wherein the one or more pre-amplifier subsets comprise a pre-amplifier specific for the pre-pre-amplifiers in the one or more pre-pre-amplifier subsets, wherein each pre-amplifier comprises binding sites for the pre-pre-amplifiers of one of the pre-pre-amplifier subsets and a plurality of binding sites for an amplifier;
(d) a set of amplifiers, wherein the amplifier set comprises one or more subsets of amplifiers specific for each pre-amplifier subset, wherein each amplifier subset comprises a plurality of amplifiers, wherein the amplifiers of one of the amplifier subsets comprise a binding site for the pre-amplifiers of one of the pre-amplifier subsets and a plurality of binding sites for a label probe; and
(e) a set of label probes, wherein the label probe set comprises one or more subsets of label probes, wherein each label probe subset is specific for one of the amplifier subsets, wherein each label probe subset comprises a plurality of label probes, wherein the label probes in each of the label probe subsets comprise a label and a binding site for the amplifiers of one of the amplifier subsets, wherein the labels in each label probe subset are distinguishable between the label probe subsets;
wherein the one or more label probe subsets in each probe subset specific for a target nucleic acid comprise at least one label or a combination of labels that is different for each probe subset.

14. The method of claim 13, wherein the set of target probes, pre-amplifiers, amplifiers and label probes each comprise two or more subsets.

15. The method of claim 13, wherein the set of target probes, pre-amplifiers, amplifiers and label probes each comprise three or more subsets.

16. The method of claim 13, wherein the set of target probes, pre-amplifiers, amplifiers and label probes each comprise four or more subsets.

17. The method of claim 14, wherein the target probe binding sites for the two or more subsets are intermingled on the target nucleic acid.

18. The method of claim 1, wherein each probe in each of the probe subsets comprises:
(a) a set of target probes, wherein the target probe set comprises a plurality of pairs of target probes that specifically hybridize to a target nucleic acid;
(b) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier;
(c) a set of pre-amplifiers, wherein the pre-amplifier set comprises a plurality of pre-amplifiers, wherein the pre-amplifiers comprise binding sites for the pre-pre-amplifiers and a plurality of binding sites for an amplifier;
(d) a set of amplifiers, wherein the amplifier set comprises a plurality of amplifiers, wherein the amplifiers comprise a binding site for the pre-amplifiers and a plurality of binding sites for a label probe or two or more distinct label probes; and
(e) a set of label probes, wherein the label probe set comprises a label probe or two or more distinct label probes, wherein each label probe comprises a label and a binding site for the amplifiers, wherein the labels in each distinct label probe are distinguishable between the distinct label probes;
wherein the amplifier in each probe subset specific for a target nucleic acid comprises a binding site for a label or a combination of two or more distinct labels that is different for each probe subset.

19. The method of claim 18, wherein the label probe set comprises two or more distinct label probes, wherein the amplifier set comprises a plurality of non-identical amplifiers, and wherein the binding sites for the two or more distinct label probes on each non-identical amplifier are in a different order on each non-identical amplifier.

20. The method of claim 1, wherein each probe in each of the probe subsets comprises:
(a) a set of target probes, wherein the target probe set comprises a plurality of pairs of target probes that specifically hybridize to a target nucleic acid;
(b) a set of pre-pre-amplifiers, wherein the pre-pre-amplifier set comprises a plurality of pre-pre-amplifiers, wherein each pre-pre-amplifier comprises binding sites for the pairs of target probes and a plurality of binding sites for a pre-amplifier;
(c) a set of pre-amplifiers, wherein the pre-amplifier set comprises a plurality of pre-amplifiers, wherein the pre-amplifiers comprise binding sites for the pre-pre-amplifiers and a plurality of binding sites for amplifiers;
(d) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for the pre-amplifiers and a plurality of binding sites for a distinct label probe; and
(e) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes;
wherein the pre-amplifier in each probe subset specific for a target nucleic acid comprises a plurality of binding sites for the amplifier comprising a binding site for the label probe or a plurality of binding sites for the two or more distinct amplifiers comprising binding sites for the two or more distinct label probes, and wherein the label of the label probe or combination of two or more distinct labels of the two or more distinct label probes is different for each probe subset.

21. The method of claim 20, wherein the plurality of amplifiers comprise two or more distinct amplifiers, and wherein the binding sites on the pre-amplifier for the distinct amplifiers are intermingled.

22. The method of claim 1, wherein each probe in each of the probe subsets comprises:
(a) a set of target probes, wherein the target probe set comprises a plurality of pairs of target probes that specifically hybridize to a target nucleic acid;
(b) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier or for two or more distinct pre-amplifiers;
(c) a set of pre-amplifiers, wherein the pre-amplifier set comprises a plurality of pre-amplifiers, wherein the plurality of pre-amplifiers comprise a pre-amplifier comprising a binding site for the pre-pre-amplifiers and a plurality of binding sites for an amplifier, or wherein the plurality of pre-amplifiers comprise two or more distinct pre-amplifiers, wherein each distinct pre-amplifier comprises a binding site for the pre-pre-amplifiers and a plurality of binding sites for a distinct amplifier;
(d) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for one of the distinct pre-amplifiers and a plurality of binding sites for a distinct label probe; and
(e) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes;
wherein the pre-pre-amplifier in each probe subset specific for a target nucleic acid comprises a plurality of binding sites for the pre-amplifier comprising a plurality of binding sites for the amplifier comprising a binding site for the label probe, or a plurality of binding sites for the two or more distinct pre-amplifiers each comprising a plurality of binding sites for one of the two or more distinct amplifiers comprising binding sites for one of the two or more distinct label probes, and wherein the label of the label probe or combination of two or more distinct labels of the two or more distinct label probes is different for each probe subset.

23. The method of claim 22, wherein the plurality of pre-amplifiers comprise two or more distinct pre-amplifiers, and wherein the binding sites on the pre-pre-amplifier for the distinct pre-amplifiers are intermingled.

24. The method of claim 1, wherein each probe in each of the probe subsets comprises:
(a) a set of target probes, wherein the target probe set comprises a plurality of pairs of target probes that specifically hybridize to a target nucleic acid;
(b) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier;
(c) a set of pre-amplifiers, wherein the pre-amplifier set comprises a plurality of pre-amplifiers, wherein the pre-amplifiers comprise binding sites for the pairs of target probes and a plurality of binding sites for an amplifier;
(d) a set of amplifiers, wherein the amplifier set comprises a plurality of amplifiers, wherein the amplifiers comprise a binding site for the pre-amplifiers and a plurality of identical binding sites for a label probe; and
(e) a set of label probes, wherein the label probe set comprises a label probe or two or more distinct label probes, wherein each label probe comprises a label and a binding site for the amplifiers, wherein the binding site for the amplifier is the same for each label probe, wherein the labels in each distinct label probe are distinguishable between the distinct label probes;
wherein the amplifier in each probe subset specific for a target nucleic acid comprises a binding site for a label probe or a combination of two or more distinct label probes that is different for each probe subset.

25. The method of claim 24, wherein the distinct labels of the two or more distinct label probes are the same in two probe subsets for two target nucleic acids and wherein the ratio of label probes in one probe subset is different than the ratio of label probes in the second probe subset, wherein a difference in ratios of distinct label probes in the first probe subset and the second probe subset distinguish the two target nucleic acids.

26. A sample comprising a cell comprising, comprising:
(A) at least one cell containing a plurality of target nucleic acids; and
(B) set of probes, wherein the set of probes comprises subsets of probes comprising a plurality detectable labels that provide unique labeling of each target nucleic acid, wherein each probe subset comprises one or more distinct labels, wherein the number and/or combination of distinct labels is unique for each target nucleic acid, and wherein at least one subset of probes is specifically hybridized to a target nucleic acid.

27. The sample of claim 26, wherein each probe in each of the probe subsets comprises:
(a) a set of target probes, wherein the target probe set comprises one or more subsets of target probes, wherein each target probe subset comprises a plurality of pairs of target probes that specifically hybridize to a target nucleic acid, wherein the target probes of a subset are hybridized to the target nucleic acid;
(b) a set of pre-amplifiers, wherein the pre-amplifier set comprises one or more subsets of pre-amplifiers, wherein the one or more pre-amplifier subsets comprise a pre-amplifier specific for each of the target probe pairs in the one or more target probe subsets, wherein each pre-amplifier comprises binding sites for the pair of target probes of one of the target probe subsets and a plurality of binding sites for an amplifier, wherein the pre-amplifiers of a subset are hybridized to the respective target probe subset;
(c) a set of amplifiers, wherein the amplifier set comprises one or more subsets of amplifiers specific for each pre-amplifier subset, wherein each amplifier subset comprises a plurality of amplifiers, wherein the amplifiers of one of the amplifier subsets comprise a binding site for the pre-amplifiers of one of the pre-amplifier subsets and a plurality of binding sites for a label probe, wherein the amplifiers of a subset are hybridized to the respective pre-amplifier subset; and
(d) a set of label probes, wherein the label probe set comprises one or more subsets of label probes, wherein each label probe subset is specific for one of the amplifier subsets, wherein each label probe subset comprises a plurality of label probes, wherein the label probes in each of the label probe subsets comprise a label and a binding site for the amplifiers of one of the amplifier subsets, wherein the labels in each label probe subset are distinguishable between the label probe subsets, wherein the label probes of a subset are hybridized to the respective amplifier subset;
wherein the one or more label probe subsets in each probe subset specific for a target nucleic acid comprise at least one label or a combination of labels that is different for each probe subset.

28. The sample of claim 27, wherein the set of target probes, pre-amplifiers, amplifiers and label probes each comprise two or more subsets.

29. The sample of claim 27, wherein the set of target probes, pre-amplifiers, amplifiers and label probes each comprise three or more subsets.

30. The sample of claim 27, wherein the set of target probes, pre-amplifiers, amplifiers and label probes each comprise four or more subsets.

31. The sample of claim 28, wherein the target probe binding sites for the two or more subsets are intermingled on the target nucleic acid.

32. The sample of claim 26, wherein each probe in each of the probe subsets comprises:
(a) a set of target probes, wherein the target probe set comprises a plurality of pairs of target probes that specifically hybridize to a target nucleic acid, wherein the target probes are hybridized to the target nucleic acid;
(b) a set of pre-amplifiers, wherein the pre-amplifier set comprises a plurality of pre-amplifiers, wherein the pre-amplifiers comprise binding sites for the pairs of target probes and a plurality of binding sites for an amplifier, wherein the pre-amplifiers are hybridized to the target probes;
(c) a set of amplifiers, wherein the amplifier set comprises a plurality of amplifiers, wherein the amplifiers comprise a binding site for the pre-amplifiers and a plurality of binding sites for a label probe or two or more distinct label probes, wherein the amplifiers are hybridized to the pre-amplifiers; and
(d) a set of label probes, wherein the label probe set comprises a label probe or two or more distinct label probes, wherein each label probe comprises a label and a binding site for the amplifiers, wherein the labels in each distinct label probe are distinguishable between the distinct label probes, wherein the label probes are hybridized to the amplifiers;
wherein the amplifier in each probe subset specific for a target nucleic acid comprises a binding site for a label or a combination of two or more distinct labels that is different for each probe subset.

33. The sample of claim 32, wherein the label probe set comprises two or more distinct label probes, wherein the amplifier set comprises a plurality of non-identical amplifiers, and wherein the binding sites for the two or more distinct label probes on each non-identical amplifier are in a different order on each non-identical amplifier.

34. The sample of claim 26, wherein each probe in each of the probe subsets comprises:
(a) a set of target probes, wherein the target probe set comprises a plurality of pairs of target probes that specifically hybridize to a target nucleic acid, wherein the target probes are hybridized to the target nucleic acid;
(b) a set of pre-amplifiers, wherein the pre-amplifier set comprises a plurality of pre-amplifiers, wherein the pre-amplifiers comprise binding sites for the pairs of target probes and a plurality of binding sites for amplifiers, wherein the pre-amplifiers are hybridized to the target probes;
(c) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for the pre-amplifiers and a plurality of binding sites for a distinct label probe, wherein the amplifiers are hybridized to the pre-amplifiers; and
(d) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes, wherein the label probes are hybridized to the amplifiers;
wherein the pre-amplifier in each probe subset specific for a target nucleic acid comprises a plurality of binding sites for the amplifier comprising a binding site for the label probe or a plurality of binding sites for the two or more distinct amplifiers comprising binding sites for the two or more distinct label probes, and wherein the label of the label probe or combination of two or more distinct labels of the two or more distinct label probes is different for each probe subset.

35. The sample of claim 34, wherein the plurality of amplifiers comprise two or more distinct amplifiers, and wherein the binding sites on the pre-amplifier for the distinct amplifiers are intermingled.

36. The sample of claim 26, wherein each probe in each of the probe subsets comprises:
(a) a set of target probes, wherein the target probe set comprises a plurality of pairs of target probes that specifically hybridize to a target nucleic acid, wherein the target probes are hybridized to the target nucleic acid;
(b) a set of pre-amplifiers, wherein the pre-amplifier set comprises a plurality of pre-amplifiers, wherein the pre-amplifiers comprise binding sites for the pairs of target probes and a plurality of binding sites for an amplifier, wherein the pre-amplifiers are hybridized to the target probes;
(c) a set of amplifiers, wherein the amplifier set comprises a plurality of amplifiers, wherein the amplifiers comprise a binding site for the pre-amplifiers and a plurality of identical binding sites for a label probe, wherein the amplifiers are hybridized to the pre-amplifiers; and
(d) a set of label probes, wherein the label probe set comprises a label probe or two or more distinct label probes, wherein each label probe comprises a label and a binding site for the amplifiers, wherein the binding site for the amplifiers is the same for each label probe, wherein the labels in each distinct label probe are distinguishable between the distinct label probes, wherein the label probes are hybridized to the amplifiers;
wherein the amplifier in each probe subset specific for a target nucleic acid comprises a binding site for a label probe or a combination of two or more distinct label probes that is different for each probe subset.

37. The sample of claim 36, wherein the distinct labels of the two or more distinct label probes are the same in two probe subsets for two target nucleic acids and wherein the ratio of label probes in one probe subset is different than the ratio of label probes in the second probe subset, wherein a difference in ratios of distinct label probes in the first probe subset and the second probe subset distinguish the two target nucleic acids.

38. The sample of claim 26, wherein each probe in each of the probe subsets comprises:
(a) a set of target probes, wherein the target probe set comprises one or more subsets of target probes, wherein each target probe subset comprises a plurality of pairs of target probes that specifically hybridize to a target nucleic acid, wherein the target probes of a subset are hybridized to the target nucleic acid;
(b) a set of pre-pre-amplifiers, wherein the pre-pre-amplifier set comprises one or more subsets of pre-pre-amplifiers, wherein the one or more pre-pre-amplifier subsets comprise a pre-pre-amplifier specific for each of the target probe pairs in the one or more target probe subsets, wherein each pre-pre-amplifier comprises binding sites for the pair of target probes of one of the target probe subsets and a plurality of binding sites for a pre-amplifier, wherein the pre-pre-amplifiers of a subset are hybridized to the respective target probe subset;
(c) a set of pre-amplifiers, wherein the pre-amplifier set comprises one or more subsets of pre-amplifiers, wherein the one or more pre-amplifier subsets comprise a pre-amplifier specific for the pre-pre-amplifiers in the one or more pre-pre-amplifier subsets, wherein each pre-amplifier comprises binding sites for the pre-pre-amplifiers of one of the pre-pre-amplifier subsets and a plurality of binding sites for an amplifier, wherein the pre-amplifiers of a subset are hybridized to the respective pre-pre-amplifier subset;
(d) a set of amplifiers, wherein the amplifier set comprises one or more subsets of amplifiers specific for each pre-amplifier subset, wherein each amplifier subset comprises a plurality of amplifiers, wherein the amplifiers of one of the amplifier subsets comprise a binding site for the pre-amplifiers of one of the pre-amplifier subsets and a plurality of binding sites for a label probe, wherein the amplifiers of a subset are hybridized to the respective pre-amplifier subset; and
(e) a set of label probes, wherein the label probe set comprises one or more subsets of label probes, wherein each label probe subset is specific for one of the amplifier subsets, wherein each label probe subset comprises a plurality of label probes, wherein the label probes in each of the label probe subsets comprise a label and a binding site for the amplifiers of one of the amplifier subsets, wherein the labels in each label probe subset are distinguishable between the label probe subsets, wherein the label probes of a subset are hybridized to the respective amplifier subset;
wherein the one or more label probe subsets in each probe subset specific for a target nucleic acid comprise at least one label or a combination of labels that is different for each probe subset.

39. The sample of claim 38, wherein the set of target probes, pre-amplifiers, amplifiers and label probes each comprise two or more subsets.

40. The sample of claim 38, wherein the set of target probes, pre-amplifiers, amplifiers and label probes each comprise three or more subsets.

41. The sample of claim 38, wherein the set of target probes, pre-amplifiers, amplifiers and label probes each comprise four or more subsets.

42. The sample of claim 39, wherein the target probe binding sites for the two or more subsets are intermingled on the target nucleic acid.

43. The sample of claim 26, wherein each probe in each of the probe subsets comprises:
(a) a set of target probes, wherein the target probe set comprises a plurality of pairs of target probes that specifically hybridize to a target nucleic acid, wherein the target probes are hybridized to the target nucleic acid;
(b) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier, wherein the pre-pre-amplifiers are hybridized to the target probes;
(c) a set of pre-amplifiers, wherein the pre-amplifier set comprises a plurality of pre-amplifiers, wherein the pre-amplifiers comprise binding sites for the pre-pre-amplifiers and a plurality of binding sites for an amplifier, wherein the pre-amplifiers are hybridized to the pre-pre-amplifiers;
(d) a set of amplifiers, wherein the amplifier set comprises a plurality of amplifiers, wherein the amplifiers comprise a binding site for the pre-amplifiers and a plurality of binding sites for a label probe or two or more distinct label probes, wherein the amplifiers are hybridized to the pre-amplifiers; and
(e) a set of label probes, wherein the label probe set comprises a label probe or two or more distinct label probes, wherein each label probe comprises a label and a binding site for the amplifiers, wherein the labels in each distinct label probe are distinguishable between the distinct label probes, wherein the label probes are hybridized to the amplifiers;
wherein the amplifier in each probe subset specific for a target nucleic acid comprises a binding site for a label or a combination of two or more distinct labels that is different for each probe subset.

44. The sample of claim 43, wherein the label probe set comprises two or more distinct label probes, wherein the amplifier set comprises a plurality of non-identical amplifiers, and wherein the binding sites for the two or more distinct label probes on each non-identical amplifier are in a different order on each non-identical amplifier.

45. The sample of claim 26, wherein each probe in each of the probe subsets comprises:
(a) a set of target probes, wherein the target probe set comprises a plurality of pairs of target probes that specifically hybridize to a target nucleic acid, wherein the target probes are hybridized to the target nucleic acid;
(b) a set of pre-pre-amplifiers, wherein the pre-pre-amplifier set comprises a plurality of pre-pre-amplifiers, wherein each pre-pre-amplifier comprises binding sites for the pairs of target probes and a plurality of binding sites for a pre-amplifier, wherein the pre-pre-amplifiers are hybridized to the target probes;
(c) a set of pre-amplifiers, wherein the pre-amplifier set comprises a plurality of pre-amplifiers, wherein the pre-amplifiers comprise binding sites for the pre-pre-amplifiers and a plurality of binding sites for amplifiers, wherein the pre-amplifiers are hybridized to the pre-pre-amplifiers;
(d) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for the pre-amplifiers and a plurality of binding sites for a distinct label probe, wherein the amplifiers are hybridized to the pre-amplifiers; and
(e) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes, wherein the label probes are hybridized to the amplifiers;
wherein the pre-amplifier in each probe subset specific for a target nucleic acid comprises a plurality of binding sites for the amplifier comprising a binding site for the label probe or a plurality of binding sites for the two or more distinct amplifiers comprising binding sites for the two or more distinct label probes, and wherein the label of the label probe or combination of two or more distinct labels of the two or more distinct label probes is different for each probe subset.

46. The sample of claim 45, wherein the plurality of amplifiers comprise two or more distinct amplifiers, and wherein the binding sites on the pre-amplifier for the distinct amplifiers are intermingled.

47. The sample of claim 26, wherein each probe in each of the probe subsets comprises:
(a) a set of target probes, wherein the target probe set comprises a plurality of pairs of target probes that specifically hybridize to a target nucleic acid, wherein the target probes are hybridized to the target nucleic acid;
(b) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier or for two or more distinct pre-amplifiers, wherein the pre-pre-amplifiers are hybridized to the target probes;
(c) a set of pre-amplifiers, wherein the pre-amplifier set comprises a plurality of pre-amplifiers, wherein the plurality of pre-amplifiers comprise a pre-amplifier comprising a binding site for the pre-pre-amplifiers and a plurality of binding sites for an amplifier, or wherein the plurality of pre-amplifiers comprise two or more distinct pre-amplifiers, wherein each distinct pre-amplifier comprises a binding site for the pre-pre-amplifiers and a plurality of binding sites for a distinct amplifier, wherein the pre-amplifiers are hybridized to the pre-pre-amplifiers;
(d) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for one of the distinct pre-amplifiers and a plurality of binding sites for a distinct label probe, wherein the amplifiers are hybridized to the pre-amplifiers; and
(e) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes, wherein the label probes are hybridized to the amplifiers;
wherein the pre-pre-amplifier in each probe subset specific for a target nucleic acid comprises a plurality of binding sites for the pre-amplifier comprising a plurality of binding sites for the amplifier comprising a binding site for the label probe, or a plurality of binding sites for the two or more distinct pre-amplifiers each comprising a plurality of binding sites for one of the two or more distinct amplifiers comprising binding sites for one of the two or more distinct label probes, and wherein the label of the label probe or combination of two or more distinct labels of the two or more distinct label probes is different for each probe subset.

48. The sample of claim 47, wherein the plurality of pre-amplifiers comprise two or more distinct pre-amplifiers, and wherein the binding sites on the pre-pre-amplifier for the distinct pre-amplifiers are intermingled.

49. The sample of claim 26, wherein each probe in each of the probe subsets comprises:
(a) a set of target probes, wherein the target probe set comprises a plurality of pairs of target probes that specifically hybridize to a target nucleic acid;
(b) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier;
(c) a set of pre-amplifiers, wherein the pre-amplifier set comprises a plurality of pre-amplifiers, wherein the pre-amplifiers comprise binding sites for the pairs of target probes and a plurality of binding sites for an amplifier;
(d) a set of amplifiers, wherein the amplifier set comprises a plurality of amplifiers, wherein the amplifiers comprise a binding site for the pre-amplifiers and a plurality of identical binding sites for a label probe; and
(e) a set of label probes, wherein the label probe set comprises a label probe or two or more distinct label probes, wherein each label probe comprises a label and a binding site for the amplifiers, wherein the binding site for the amplifier is the same for each label probe, wherein the labels in each distinct label probe are distinguishable between the distinct label probes;
wherein the amplifier in each probe subset specific for a target nucleic acid comprises a binding site for a label probe or a combination of two or more distinct label probes that is different for each probe subset.

50. The method of claim 49, wherein the distinct labels of the two or more distinct label probes are the same in two probe subsets for two target nucleic acids and wherein the ratio of label probes in one probe subset is different than the ratio of label probes in the second probe subset, wherein a difference in ratios of distinct label probes in the first probe subset and the second probe subset distinguish the two target nucleic acids.

51. A slide comprising:
(A) a slide having immobilized thereon at least one cell containing a plurality of target nucleic acids; and
(B) set of probes, wherein the set of probes comprises subsets of probes comprising a plurality detectable labels that provide unique labeling of each target nucleic acid, wherein each probe subset comprises one or more distinct labels, wherein the number and/or combination of distinct labels is unique for each target nucleic acid, and wherein at least one subset of probes is specifically hybridized to a target nucleic acid.

52. The slide of claim 51, wherein each probe in each of the probe subsets comprises:
(a) a set of target probes, wherein the target probe set comprises one or more subsets of target probes, wherein each target probe subset comprises a plurality of pairs of target probes that specifically hybridize to a target nucleic acid, wherein the target probes of a subset are hybridized to the target nucleic acid;
(b) a set of pre-amplifiers, wherein the pre-amplifier set comprises one or more subsets of pre-amplifiers, wherein the one or more pre-amplifier subsets comprise a pre-amplifier specific for each of the target probe pairs in the one or more target probe subsets, wherein each pre-amplifier comprises binding sites for the pair of target probes of one of the target probe subsets and a plurality of binding sites for an amplifier, wherein the pre-amplifiers of a subset are hybridized to the respective target probe subset;
(c) a set of amplifiers, wherein the amplifier set comprises one or more subsets of amplifiers specific for each pre-amplifier subset, wherein each amplifier subset comprises a plurality of amplifiers, wherein the amplifiers of one of the amplifier subsets comprise a binding site for the pre-amplifiers of one of the pre-amplifier subsets and a plurality of binding sites for a label probe, wherein the amplifiers of a subset are hybridized to the respective pre-amplifier subset; and
(d) a set of label probes, wherein the label probe set comprises one or more subsets of label probes, wherein each label probe subset is specific for one of the amplifier subsets, wherein each label probe subset comprises a plurality of label probes, wherein the label probes in each of the label probe subsets comprise a label and a binding site for the amplifiers of one of the amplifier subsets, wherein the labels in each label probe subset are distinguishable between the label probe subsets, wherein the label probes of a subset are hybridized to the respective amplifier subset;
wherein the one or more label probe subsets in each probe subset specific for a target nucleic acid comprise at least one label or a combination of labels that is different for each probe subset.

53. The slide of claim 52, wherein the set of target probes, pre-amplifiers, amplifiers and label probes each comprise two or more subsets.

54. The slide of claim 52, wherein the set of target probes, pre-amplifiers, amplifiers and label probes each comprise three or more subsets.

55. The slide of claim 52, wherein the set of target probes, pre-amplifiers, amplifiers and label probes each comprise four or more subsets.

56. The slide of claim 53, wherein the target probe binding sites for the two or more subsets are intermingled on the target nucleic acid.

57. The slide of claim 51, wherein each probe in each of the probe subsets comprises:
(a) a set of target probes, wherein the target probe set comprises a plurality of pairs of target probes that specifically hybridize to a target nucleic acid, wherein the target probes are hybridized to the target nucleic acid;
(b) a set of pre-amplifiers, wherein the pre-amplifier set comprises a plurality of pre-amplifiers, wherein the pre-amplifiers comprise binding sites for the pairs of target probes and a plurality of binding sites for an amplifier, wherein the pre-amplifiers are hybridized to the target probes;
(c) a set of amplifiers, wherein the amplifier set comprises a plurality of amplifiers, wherein the amplifiers comprise a binding site for the pre-amplifiers and a plurality of binding sites for a label probe or two or more distinct label probes, wherein the amplifiers are hybridized to the pre-amplifiers; and
(d) a set of label probes, wherein the label probe set comprises a label probe or two or more distinct label probes, wherein each label probe comprises a label and a binding site for the amplifiers, wherein the labels in each distinct label probe are distinguishable between the distinct label probes, wherein the label probes are hybridized to the amplifiers;
wherein the amplifier in each probe subset specific for a target nucleic acid comprises a binding site for a label or a combination of two or more distinct labels that is different for each probe subset.

58. The slide of claim 57, wherein the label probe set comprises two or more distinct label probes, wherein the amplifier set comprises a plurality of non-identical amplifiers, and wherein the binding sites for the two or more distinct label probes on each non-identical amplifier are in a different order on each non-identical amplifier.

59. The slide of claim 51, wherein each probe in each of the probe subsets comprises:
(a) a set of target probes, wherein the target probe set comprises a plurality of pairs of target probes that specifically hybridize to a target nucleic acid, wherein the target probes are hybridized to the target nucleic acid;
(b) a set of pre-amplifiers, wherein the pre-amplifier set comprises a plurality of pre-amplifiers, wherein the pre-amplifiers comprise binding sites for the pairs of target probes and a plurality of binding sites for amplifiers, wherein the pre-amplifiers are hybridized to the target probes;
(c) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for the pre-amplifiers and a plurality of binding sites for a distinct label probe, wherein the amplifiers are hybridized to the pre-amplifiers; and
(d) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes, wherein the label probes are hybridized to the amplifiers;
wherein the pre-amplifier in each probe subset specific for a target nucleic acid comprises a plurality of binding sites for the amplifier comprising a binding site for the label probe or a plurality of binding sites for the two or more distinct amplifiers comprising binding sites for the two or more distinct label probes, and wherein the label of the label probe or combination of two or more distinct labels of the two or more distinct label probes is different for each probe subset.

60. The slide of claim 59, wherein the plurality of amplifiers comprise two or more distinct amplifiers, and wherein the binding sites on the pre-amplifier for the distinct amplifiers are intermingled.

61. The slide of claim 51, wherein each probe in each of the probe subsets comprises:
(a) a set of target probes, wherein the target probe set comprises a plurality of pairs of target probes that specifically hybridize to a target nucleic acid, wherein the target probes are hybridized to the target nucleic acid;
(b) a set of pre-amplifiers, wherein the pre-amplifier set comprises a plurality of pre-amplifiers, wherein the pre-amplifiers comprise binding sites for the pairs of target probes and a plurality of binding sites for an amplifier, wherein the pre-amplifiers are hybridized to the target probes;
(c) a set of amplifiers, wherein the amplifier set comprises a plurality of amplifiers, wherein the amplifiers comprise a binding site for the pre-amplifiers and a plurality of identical binding sites for a label probe, wherein the amplifiers are hybridized to the pre-amplifiers; and
(d) a set of label probes, wherein the label probe set comprises a label probe or two more distinct label probes, wherein each label probe comprises a label and a binding site for the amplifiers, wherein the binding site for the amplifier is the same for each label probe, wherein the labels in each distinct label probe are distinguishable between the distinct label probes, wherein the label probes are hybridized to the amplifiers;
wherein the amplifier in each probe subset specific for a target nucleic acid comprises a binding site for a label probe or a combination of two or more distinct label probes that is different for each probe subset.

62. The slide of claim 61, wherein the distinct labels of the two or more distinct label probes are the same in two probe subsets for two target nucleic acids and wherein the ratio of label probes in one probe subset is different than the ratio of label probes in the second probe subset, wherein a difference in ratios of distinct label probes in the first probe subset and the second probe subset distinguish the two target nucleic acids.

63. The slide of claim 51, wherein each probe in each of the probe subsets comprises:
(a) a set of target probes, wherein the target probe set comprises one or more subsets of target probes, wherein each target probe subset comprises a plurality of pairs of target probes that specifically hybridize to a target nucleic acid, wherein the target probes of a subset are hybridized to the target nucleic acid;
(b) a set of pre-pre-amplifiers, wherein the pre-pre-amplifier set comprises one or more subsets of pre-pre-amplifiers, wherein the one or more pre-pre-amplifier subsets comprise a pre-pre-amplifier specific for each of the target probe pairs in the one or more target probe subsets, wherein each pre-pre-amplifier comprises binding sites for the pair of target probes of one of the target probe subsets and a plurality of binding sites for a pre-amplifier, wherein the pre-pre-amplifiers of a subset are hybridized to the respective target probe subset;
(c) a set of pre-amplifiers, wherein the pre-amplifier set comprises one or more subsets of pre-amplifiers, wherein the one or more pre-amplifier subsets comprise a pre-amplifier specific for the pre-pre-amplifiers in the one or more pre-pre-amplifier subsets, wherein each pre-amplifier comprises binding sites for the pre-pre-amplifiers of one of the pre-pre-amplifier subsets and a plurality of binding sites for an amplifier, wherein the pre-amplifiers of a subset are hybridized to the respective pre-pre-amplifier subset;
(d) a set of amplifiers, wherein the amplifier set comprises one or more subsets of amplifiers specific for each pre-amplifier subset, wherein each amplifier subset comprises a plurality of amplifiers, wherein the amplifiers of one of the amplifier subsets comprise a binding site for the pre-amplifiers of one of the pre-amplifier subsets and a plurality of binding sites for a label probe, wherein the amplifiers of a subset are hybridized to the respective pre-amplifier subset; and
(e) a set of label probes, wherein the label probe set comprises one or more subsets of label probes, wherein each label probe subset is specific for one of the amplifier subsets, wherein each label probe subset comprises a plurality of label probes, wherein the label probes in each of the label probe subsets comprise a label and a binding site for the amplifiers of one of the amplifier subsets, wherein the labels in each label probe subset are distinguishable between the label probe subsets, wherein the label probes of a subset are hybridized to the respective amplifier subset;
wherein the one or more label probe subsets in each probe subset specific for a target nucleic acid comprise at least one label or a combination of labels that is different for each probe subset.

64. The slide of claim 63, wherein the set of target probes, pre-amplifiers, amplifiers and label probes each comprise two or more subsets.

65. The slide of claim 63, wherein the set of target probes, pre-amplifiers, amplifiers and label probes each comprise three or more subsets.

66. The slide of claim 63, wherein the set of target probes, pre-amplifiers, amplifiers and label probes each comprise four or more subsets.

67. The slide of claim 64, wherein the target probe binding sites for the two or more subsets are intermingled on the target nucleic acid.

68. The slide of claim 51, wherein each probe in each of the probe subsets comprises:
(a) a set of target probes, wherein the target probe set comprises a plurality of pairs of target probes that specifically hybridize to a target nucleic acid, wherein the target probes are hybridized to the target nucleic acid;
(b) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier, wherein the pre-pre-amplifiers are hybridized to the target probes;
(c) a set of pre-amplifiers, wherein the pre-amplifier set comprises a plurality of pre-amplifiers, wherein the pre-amplifiers comprise binding sites for the pre-pre-amplifiers and a plurality of binding sites for an amplifier, wherein the pre-amplifiers are hybridized to the pre-pre-amplifiers;
(d) a set of amplifiers, wherein the amplifier set comprises a plurality of amplifiers, wherein the amplifiers comprise a binding site for the pre-amplifiers and a plurality of binding sites for a label probe or two or more distinct label probes, wherein the amplifiers are hybridized to the pre-amplifiers; and
(e) a set of label probes, wherein the label probe set comprises a label probe or two or more distinct label probes, wherein each label probe comprises a label and a binding site for the amplifiers, wherein the labels in each distinct label probe are distinguishable between the distinct label probes, wherein the label probes are hybridized to the amplifiers;
wherein the amplifier in each probe subset specific for a target nucleic acid comprises a binding site for a label or a combination of two or more distinct labels that is different for each probe subset.

69. The slide of claim 68, wherein the label probe set comprises two or more distinct label probes, wherein the amplifier set comprises a plurality of non-identical amplifiers, and wherein the binding sites for the two or more distinct label probes on each non-identical amplifier are in a different order on each non-identical amplifier.

70. The slide of claim 51, wherein each probe in each of the probe subsets comprises:
(a) a set of target probes, wherein the target probe set comprises a plurality of pairs of target probes that specifically hybridize to a target nucleic acid, wherein the target probes are hybridized to the target nucleic acid;
(b) a set of pre-pre-amplifiers, wherein the pre-pre-amplifier set comprises a plurality of pre-pre-amplifiers, wherein each pre-pre-amplifier comprises binding sites for the pairs of target probes and a plurality of binding sites for a pre-amplifier, wherein the pre-pre-amplifiers are hybridized to the target probes;
(c) a set of pre-amplifiers, wherein the pre-amplifier set comprises a plurality of pre-amplifiers, wherein the pre-amplifiers comprise binding sites for the pre-pre-amplifiers and a plurality of binding sites for amplifiers, wherein the pre-amplifiers are hybridized to the pre-pre-amplifiers;
(d) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for the pre-amplifiers and a plurality of binding sites for a distinct label probe, wherein the amplifiers are hybridized to the pre-amplifiers; and
(e) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes, wherein the label probes are hybridized to the amplifiers;
wherein the pre-amplifier in each probe subset specific for a target nucleic acid comprises a plurality of binding sites for the amplifier comprising a binding site for the label probe or a plurality of binding sites for the two or more distinct amplifiers comprising binding sites for the two or more distinct label probes, and wherein the label of the label probe or combination of two or more distinct labels of the two or more distinct label probes is different for each probe subset.

71. The slide of claim 70, wherein the plurality of amplifiers comprise two or more distinct amplifiers, and wherein the binding sites on the pre-amplifier for the distinct amplifiers are intermingled.

72. The slide of claim 51, wherein each probe in each of the probe subsets comprises:
(a) a set of target probes, wherein the target probe set comprises a plurality of pairs of target probes that specifically hybridize to a target nucleic acid, wherein the target probes are hybridized to the target nucleic acid;
(b) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier or for two or more distinct pre-amplifiers, wherein the pre-pre-amplifiers are hybridized to the target probes;
(c) a set of pre-amplifiers, wherein the pre-amplifier set comprises a plurality of pre-amplifiers, wherein the plurality of pre-amplifiers comprise a pre-amplifier comprising a binding site for the pre-pre-amplifiers and a plurality of binding sites for an amplifier, or wherein the plurality of pre-amplifiers comprise two or more distinct pre-amplifiers, wherein each distinct pre-amplifier comprises a binding site for the pre-pre-amplifiers and a plurality of binding sites for a distinct amplifier, wherein the pre-amplifiers are hybridized to the pre-pre-amplifiers;
(d) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for one of the distinct pre-amplifiers and a plurality of binding sites for a distinct label probe, wherein the amplifiers are hybridized to the pre-amplifiers; and
(e) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes, wherein the label probes are hybridized to the amplifiers;
wherein the pre-pre-amplifier in each probe subset specific for a target nucleic acid comprises a plurality of binding sites for the pre-amplifier comprising a plurality of binding sites for the amplifier comprising a binding site for the label probe, or a plurality of binding sites for the two or more distinct pre-amplifiers each comprising a plurality of binding sites for one of the two or more distinct amplifiers comprising binding sites for one of the two or more distinct label probes, and wherein the label of the label probe or combination of two or more distinct labels of the two or more distinct label probes is different for each probe subset.

73. The slide of claim 72, wherein the plurality of pre-amplifiers comprise two or more distinct pre-amplifiers, and wherein the binding sites on the pre-pre-amplifier for the distinct pre-amplifiers are intermingled.

74. The slide of claim 51, wherein each probe in each of the probe subsets comprises:
(a) a set of target probes, wherein the target probe set comprises a plurality of pairs of target probes that specifically hybridize to a target nucleic acid;
(b) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier;
(c) a set of pre-amplifiers, wherein the pre-amplifier set comprises a plurality of pre-amplifiers, wherein the pre-amplifiers comprise binding sites for the pairs of target probes and a plurality of binding sites for an amplifier;
(d) a set of amplifiers, wherein the amplifier set comprises a plurality of amplifiers, wherein the amplifiers comprise a binding site for the pre-amplifiers and a plurality of identical binding sites for a label probe; and
(e) a set of label probes, wherein the label probe set comprises a label probe or two or more distinct label probes, wherein each label probe comprises a label and a binding site for the amplifiers, wherein the binding site for the amplifier is the same for each label probe, wherein the labels in each distinct label probe are distinguishable between the distinct label probes;
wherein the amplifier in each probe subset specific for a target nucleic acid comprises a binding site for a label probe or a combination of two or more distinct label probes that is different for each probe subset.

75. The slide of claim 74, wherein the distinct labels of the two or more distinct label probes are the same in two probe subsets for two target nucleic acids and wherein the ratio of label probes in one probe subset is different than the ratio of label probes in the second probe subset, wherein a difference in ratios of distinct label probes in the first probe subset and the second probe subset distinguish the two target nucleic acids.

76. A kit for multiplex detection of a plurality of target nucleic acids in a cell, comprising a set of probes, wherein the set of probes comprises subsets of probes comprising a plurality detectable labels that provide unique labeling of each target nucleic acid, wherein each probe subset comprises one or more distinct labels, wherein the number and/or combination of distinct labels is unique for each target nucleic acid.

77. The kit of claim 76, wherein each probe in each of the probe subsets comprises:
(a) a set of pre-amplifiers, wherein the pre-amplifier set comprises one or more subsets of pre-amplifiers, wherein the one or more pre-amplifier subsets comprise a pre-amplifier specific for each of the target probe pairs in the one or more target probe subsets, wherein each pre-amplifier comprises binding sites for the pair of target probes of one of the target probe subsets and a plurality of binding sites for an amplifier;
(b) a set of amplifiers, wherein the amplifier set comprises one or more subsets of amplifiers specific for each pre-amplifier subset, wherein each amplifier subset comprises a plurality of amplifiers, wherein the amplifiers of one of the amplifier subsets comprise a binding site for the pre-amplifiers of one of the pre-amplifier subsets and a plurality of binding sites for a label probe; and
(c) a set of label probes, wherein the label probe set comprises one or more subsets of label probes, wherein each label probe subset is specific for one of the amplifier subsets, wherein each label probe subset comprises a plurality of label probes, wherein the label probes in each of the label probe subsets comprise a label and a binding site for the amplifiers of one of the amplifier subsets, wherein the labels in each label probe subset are distinguishable between the label probe subsets;
wherein the one or more label probe subsets in each probe subset specific for a target nucleic acid comprise at least one label or a combination of labels that is different for each probe subset.

78. The kit of claim 77, wherein the kit comprises a set of target probes, wherein the target probe set comprises one or more subsets of target probes, wherein each target probe subset comprises a plurality of pairs of target probes that specifically hybridize to a target nucleic acid.

79. The kit of claim 77 or 78, wherein the set of target probes, pre-amplifiers, amplifiers and label probes each comprise two or more subsets.

80. The kit of claim 77 or 78, wherein the set of target probes, pre-amplifiers, amplifiers and label probes each comprise three or more subsets.

81. The kit of claim 77 or 78, wherein the set of target probes, pre-amplifiers, amplifiers and label probes each comprise four or more subsets.

82. The kit of claim 79, wherein the target probe binding sites for the two or more subsets are intermingled on the target nucleic acid.

83. The kit of claim 76, wherein each probe in each of the probe subsets comprises:
(a) a set of pre-amplifiers, wherein the pre-amplifier set comprises a plurality of pre-amplifiers, wherein the pre-amplifiers comprise binding sites for the pairs of target probes and a plurality of binding sites for an amplifier;
(b) a set of amplifiers, wherein the amplifier set comprises a plurality of amplifiers, wherein the amplifiers comprise a binding site for the pre-amplifiers and a plurality of binding sites for a label probe or two or more distinct label probes; and
(c) a set of label probes, wherein the label probe set comprises a label probe or two or more distinct label probes, wherein each label probe comprises a label and a binding site for the amplifiers, wherein the labels in each distinct label probe are distinguishable between the distinct label probes;
wherein the amplifier in each probe subset specific for a target nucleic acid comprises a binding site for a label or a combination of two or more distinct labels that is different for each probe subset.

84. The kit of claim 83, wherein the kit comprises a set of target probes, wherein the target probe set comprises a plurality of pairs of target probes that specifically hybridize to a target nucleic acid.

85. The kit of claim 83 or 84, wherein the label probe set comprises two or more distinct label probes, wherein the amplifier set comprises a plurality of non-identical amplifiers, and wherein the binding sites for the two or more distinct label probes on each non-identical amplifier are in a different order on each non-identical amplifier.

86. The kit of claim 76, wherein each probe in each of the probe subsets comprises:
(a) a set of pre-amplifiers, wherein the pre-amplifier set comprises a plurality of pre-amplifiers, wherein the pre-amplifiers comprise binding sites for the pairs of target probes and a plurality of binding sites for amplifiers;
(b) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for the pre-amplifiers and a plurality of binding sites for a distinct label probe; and
(c) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes;
wherein the pre-amplifier in each probe subset specific for a target nucleic acid comprises a plurality of binding sites for the amplifier comprising a binding site for the label probe or a plurality of binding sites for the two or more distinct amplifiers comprising binding sites for the two or more distinct label probes, and wherein the label of the label probe or combination of two or more distinct labels of the two or more distinct label probes is different for each probe subset.

87. The kit of claim 86, wherein the kit comprises a set of target probes, wherein the target probe set comprises a plurality of pairs of target probes that specifically hybridize to a target nucleic acid.

88. The kit of claim 86 or 87, wherein the plurality of amplifiers comprise two or more distinct amplifiers, and wherein the binding sites on the pre-amplifier for the distinct amplifiers are intermingled.

89. The kit of claim 76, wherein each probe in each of the probe subsets comprises:
(a) a set of pre-amplifiers, wherein the pre-amplifier set comprises a plurality of pre-amplifiers, wherein the pre-amplifiers comprise binding sites for the pairs of target probes and a plurality of binding sites for an amplifier;
(b) a set of amplifiers, wherein the amplifier set comprises a plurality of amplifiers, wherein the amplifiers comprise a binding site for the pre-amplifiers and a plurality of identical binding sites for a label probe; and
(c) a set of label probes, wherein the label probe set comprises a label probe or two or more distinct label probes, wherein each label probe comprises a label and a binding site for the amplifiers, wherein the binding site for the amplifier is the same for each label probe, wherein the labels in each distinct label probe are distinguishable between the distinct label probes; and wherein the label in each label probe subset is distinct from the label in another label probe subset;
wherein the amplifier in each probe subset specific for a target nucleic acid comprises a binding site for a label probe or a combination of two or more distinct label probes that is different for each probe subset.

90. The kit of claim 89, wherein the distinct labels of the two or more distinct label probes are the same in two probe subsets for two target nucleic acids and wherein the ratio of label probes in one probe subset is different than the ratio of label probes in the second probe subset, wherein a difference in ratios of distinct label probes in the first probe subset and the second probe subset distinguish the two target nucleic acids.

91. The kit of claim 89 or 90, wherein the kit comprises a set of target probes, wherein the target probe set comprises a plurality of pairs of target probes that specifically hybridize to a target nucleic acid.

92. The kit of claim 76, wherein each probe in each of the probe subsets comprises:
(a) a set of pre-pre-amplifiers, wherein the pre-pre-amplifier set comprises one or more subsets of pre-pre-amplifiers, wherein the one or more pre-pre-amplifier subsets comprise a pre-pre-amplifier specific for each of the target probe pairs in the one or more target probe subsets, wherein each pre-pre-amplifier comprises binding sites for the pair of target probes of one of the target probe subsets and a plurality of binding sites for a pre-amplifier;
(b) a set of pre-amplifiers, wherein the pre-amplifier set comprises one or more subsets of pre-amplifiers, wherein the one or more pre-amplifier subsets comprise a pre-amplifier specific for the pre-pre-amplifiers in the one or more pre-pre-amplifier subsets, wherein each pre-amplifier comprises binding sites for the pre-pre-amplifiers of one of the pre-pre-amplifier subsets and a plurality of binding sites for an amplifier;
(c) a set of amplifiers, wherein the amplifier set comprises one or more subsets of amplifiers specific for each pre-amplifier subset, wherein each amplifier subset comprises a plurality of amplifiers, wherein the amplifiers of one of the amplifier subsets comprise a binding site for the pre-amplifiers of one of the pre-amplifier subsets and a plurality of binding sites for a label probe; and
(d) a set of label probes, wherein the label probe set comprises one or more subsets of label probes, wherein each label probe subset is specific for one of the amplifier subsets, wherein each label probe subset comprises a plurality of label probes, wherein the label probes in each of the label probe subsets comprise a label and a binding site for the amplifiers of one of the amplifier subsets, wherein the labels in each label probe subset are distinguishable between the label probe subsets;
wherein the one or more label probe subsets in each probe subset specific for a target nucleic acid comprise at least one label or a combination of labels that is different for each probe subset.

93. The kit of claim 92, wherein the kit comprises a set of target probes, wherein the target probe set comprises one or more subsets of target probes, wherein each target probe subset comprises a plurality of pairs of target probes that specifically hybridize to a target nucleic acid.

94. The kit of claim 92 or 93, wherein the set of target probes, pre-amplifiers, amplifiers and label probes each comprise two or more subsets.

95. The kit of claim 92 or 93, wherein the set of target probes, pre-amplifiers, amplifiers and label probes each comprise three or more subsets.

96. The kit of claim 92 or 93, wherein the set of target probes, pre-amplifiers, amplifiers and label probes each comprise four or more subsets.

97. The kit of claim 94, wherein the target probe binding sites for the two or more subsets are intermingled on the target nucleic acid.

98. The kit of claim 76, wherein each probe in each of the probe subsets comprises:
(a) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier;
(b) a set of pre-amplifiers, wherein the pre-amplifier set comprises a plurality of pre-amplifiers, wherein the pre-amplifiers comprise binding sites for the pre-pre-amplifiers and a plurality of binding sites for an amplifier;
(c) a set of amplifiers, wherein the amplifier set comprises a plurality of amplifiers, wherein the amplifiers comprise a binding site for the pre-amplifiers and a plurality of binding sites for a label probe or two or more distinct label probes; and
(d) a set of label probes, wherein the label probe set comprises a label probe or two or more distinct label probes, wherein each label probe comprises a label and a binding site for the amplifiers, wherein the labels in each distinct label probe are distinguishable between the distinct label probes;
wherein the amplifier in each probe subset specific for a target nucleic acid comprises a binding site for a label or a combination of two or more distinct labels that is different for each probe subset.

99. The kit of claim 98, wherein the kit comprises a set of target probes, wherein the target probe set comprises a plurality of pairs of target probes that specifically hybridize to a target nucleic acid.

100. The kit of claim 98 or 99, wherein the label probe set comprises two or more distinct label probes, wherein the amplifier set comprises a plurality of non-identical amplifiers, and wherein the binding sites for the two or more distinct label probes on each non-identical amplifier are in a different order on each non-identical amplifier.

101. The kit of claim 76, wherein each probe in each of the probe subsets comprises:
(a) a set of pre-pre-amplifiers, wherein the pre-pre-amplifier set comprises a plurality of pre-pre-amplifiers, wherein each pre-pre-amplifier comprises binding sites for the pairs of target probes and a plurality of binding sites for a pre-amplifier;
(b) a set of pre-amplifiers, wherein the pre-amplifier set comprises a plurality of pre-amplifiers, wherein the pre-amplifiers comprise binding sites for the pre-pre-amplifiers and a plurality of binding sites for amplifiers;
(c) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for the pre-amplifiers and a plurality of binding sites for a distinct label probe; and
(d) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes;
wherein the pre-amplifier in each probe subset specific for a target nucleic acid comprises a plurality of binding sites for the amplifier comprising a binding site for the label probe or a plurality of binding sites for the two or more distinct amplifiers comprising binding sites for the two or more distinct label probes, and wherein the label of the label probe or combination of two or more distinct labels of the two or more distinct label probes is different for each probe subset.

102. The kit of claim 101, wherein the kit comprises a set of target probes, wherein the target probe set comprises a plurality of pairs of target probes that specifically hybridize to a target nucleic acid.

103. The kit of claim 101 or 102, wherein the plurality of amplifiers comprise two or more distinct amplifiers, and wherein the binding sites on the pre-amplifier for the distinct amplifiers are intermingled.

104. The kit of claim 76, wherein each probe in each of the probe subsets comprises:
(a) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier or for two or more distinct pre-amplifiers;
(b) a set of pre-amplifiers, wherein the pre-amplifier set comprises a plurality of pre-amplifiers, wherein the plurality of pre-amplifiers comprise a pre-amplifier comprising a binding site for the pre-pre-amplifiers and a plurality of binding sites for an amplifier, or wherein the plurality of pre-amplifiers comprise two or more distinct pre-amplifiers, wherein each distinct pre-amplifier comprises a binding site for the pre-pre-amplifiers and a plurality of binding sites for a distinct amplifier;
(c) a set of amplifiers, wherein the amplifier set comprises a plurality of amplifiers, wherein the plurality of amplifiers comprise an amplifier comprising a binding site for the pre-amplifiers and a plurality of binding sites for a label probe, or wherein the plurality of amplifiers comprise two or more distinct amplifiers, wherein each distinct amplifier comprises a binding site for one of the distinct pre-amplifiers and a plurality of binding sites for a distinct label probe; and
(d) a set of label probes, wherein the label probe set comprises a label probe or two or more distinct label probes, wherein the label probe comprises a label and a binding site for the amplifier, or wherein the two or more distinct label probes comprise a label and a binding site for the two or more distinct amplifiers, wherein the labels on each distinct label probe are distinguishable between the distinct label probes;
wherein the pre-pre-amplifier in each probe subset specific for a target nucleic acid comprises a plurality of binding sites for the pre-amplifier comprising a plurality of binding sites for the amplifier comprising a binding site for the label probe, or a plurality of binding sites for the two or more distinct pre-amplifiers each comprising a plurality of binding sites for one of the two or more distinct amplifiers comprising binding sites for one of the two or more distinct label probes, and wherein the label of the label probe or combination of two or more distinct labels of the two or more distinct label probes is different for each probe subset.

105. The kit of claim 104, wherein the kit comprises a set of target probes, wherein the target probe set comprises a plurality of pairs of target probes that specifically hybridize to a target nucleic acid.

106. The kit of claim 104 or 105, wherein the plurality of pre-amplifiers comprise two or more distinct pre-amplifiers, and wherein the binding sites on the pre-pre-amplifier for the distinct pre-amplifiers are intermingled.

107. The kit of claim 76, wherein each probe in each of the probe subsets comprises:
(a) a set of target probes, wherein the target probe set comprises a plurality of pairs of target probes that specifically hybridize to a target nucleic acid;
(b) a set of pre-pre-amplifiers, wherein the pre-pre-amplifier set comprises a plurality of pre-pre-amplifiers, wherein the pre-pre-amplifiers comprise binding sites for the pairs of target probes and a plurality of binding sites for a pre-amplifier;
(c) a set of pre-amplifiers, wherein the pre-amplifier set comprises a plurality of pre-amplifiers, wherein the pre-amplifiers comprise binding sites for the pairs of target probes and a plurality of binding sites for an amplifier;
(d) a set of amplifiers, wherein the amplifier set comprises a plurality of amplifiers, wherein the amplifiers comprise a binding site for the pre-amplifiers and a plurality of identical binding sites for a label probe; and
(e) a set of label probes, wherein the label probe set comprises a label probe or two or more distinct label probes, wherein each label probe comprises a label and a binding site for the amplifiers, wherein the binding site for the amplifier is the same for each label probe, wherein the labels in each distinct label probe are distinguishable between the distinct label probes;
wherein the amplifier in each probe subset specific for a target nucleic acid comprises a binding site for a label probe or a combination of two or more distinct label probes that is different for each probe subset.

108. The kit of claim 107, wherein the distinct labels of the two or more distinct label probes are the same in two probe subsets for two target nucleic acids and wherein the ratio of label probes in one probe subset is different than the ratio of label probes in the second probe subset, wherein a difference in ratios of distinct label probes in the first probe subset and the second probe subset distinguish the two target nucleic acids.

109. The kit of any one of claims 76-108, wherein the kit comprises a reagent for fixing and/or permeabilizing cells.
